(19) United States
(12) Patent Application Publication
(10) Pub. No.: US 2025/0195653 A1
(43) Pub. Date: Jun. 19, 2025

(54) MULTIFUNCTIONAL NATURAL KILLER (NK) CELL ENGAGER COMBINATION THERAPY FOR TREATING HEMATOLOGICAL NEOPLASTIC DISORDERS

(71) Applicant: SANOFI, Paris (FR)

(72) Inventors: Giovanni ABBADESSA, Cambridge, MA (US); Marielle CHIRON, Paris (FR); Paula FRAENKEL, Cambridge, MA (US); Angela VIRONE-ODDOS, Paris (FR)

(21) Appl. No.: 18/961,791

(22) Filed: Nov. 27, 2024

(30) Foreign Application Priority Data
Nov. 28, 2023 (EP) 23307073.9
Jul. 31, 2024 (EP) 24306298.1

(51) Int. Cl. A61K 40/15; A61K 31/635; A61K 31/706; A61K 40/42; A61P 35/02; C07K 16/28

(52) U.S. Cl. CPC A61K 40/15; A61K 31/635; A61K 31/706; A61K 40/421; A61K 40/4217; A61P 35/02; C07K 16/2803; C07K 16/2866

(57) ABSTRACT

The present disclosure relates to methods for treating or preventing a leukemia or a myelodysplastic syndrome in a subject in need thereof, said method comprising administering to the subject an effective amount of a combination comprising: (i) a binding protein comprising a first antigen binding domain (ABD) with binding specificity to CD123 and a second (ABD) with binding specificity to NKp46; and one or both of: (ii) a BCL-2 inhibitor, and (iii) a DNA hypomethylating agent.

### RELATED APPLICATIONS

This application claims priority to European Patent Application Nos. 23307073.9, filed Nov. 28, 2023, and 24306298.1, filed Jul. 31, 2024, the entire disclosures of which are hereby incorporated herein by reference.

### SEQUENCE LISTING

The instant application contains a Sequence Listing which has been submitted herewith and is hereby incorporated by reference in its entirety. Said .xml copy, created on Nov. 26, 2024, is named 759035_SA9-507_ST26.xml, and is 113,273 bytes in size.

### BACKGROUND

Acute myeloid leukemia (AML), B cell acute lymphoblastic leukemia (B-ALL), and myelodysplastic syndromes (MDS) are heterogeneous clonal neoplastic diseases, which are thought to arise from subpopulations of leukemic stem cells, which tend to be resistant to conventional chemotherapy, and which may be further responsible for disease relapse.

There is an urgent need for methods of treatment for patients who are ineligible for or have exhausted standard therapeutic options.

### SUMMARY

This disclosure provides methods of treating for treating or preventing a leukemia (e.g., AML, B-ALL), a myelodysplastic syndrome (MDS) or other hematological neoplastic disorder in a subject in need thereof, said method comprising administering to the subject an effective amount of a combination comprising: (i) a multifunctional binding protein comprising a first antigen binding domain (ABD) with binding specificity to CD123 (an antigen of interest on tumoral target cells) and a second (ABD) with binding specificity to NKp46 (a surface biomarker on immune NK cells); and one or both of: (ii) a BCL-2 inhibitor, and (iii) a DNA hypomethylating agent.

In some embodiments, the binding protein further comprises all or part of an immunoglobulin Fc variant region or variant thereof. In some embodiments, all or part of the Fc region or variant thereof binds to a human Fc-γ receptor.

In some embodiments, the first ABD binds specifically to human CD123 and comprises:
(i) a heavy chain variable domain (VH1) comprising a CDR-H1, H2, and H3 corresponding to the amino acid sequences of SEQ ID NO: 1, 2, and 3, respectively or corresponding to the amino acid sequences of SEQ ID NO: 4, 5, and 6, respectively; and
(ii) a light chain variable domain (VL1) comprising a CDR-L1, L2, and L3 corresponding to the amino acid sequences of SEQ ID NO: 7, 8, and 9, respectively or corresponding to the amino acid sequences of SEQ ID NO: 10, 11, and 12, respectively.

In some embodiments, a) the V_{H1} comprises an amino acid sequence that is at least about 90% identical to the amino acid sequence of SEQ ID NO: 41, and wherein the V_{L1} comprises an amino acid sequence that is at least about 90% identical to the amino acid sequence of SEQ ID NO: 43; or b) the V_{H1} comprises an amino acid sequence that is at least about 90% identical to the amino acid sequence of SEQ ID NO: 42, and wherein the VL1 comprises an amino acid sequence that is at least about 90% identical to the amino acid sequence of SEQ ID NO: 44.

In some embodiments, a) the V_{H1} comprises an amino acid sequence of SEQ ID NO: 41, and wherein the V_{L1} comprises an amino acid sequence of SEQ ID NO: 43; or b) the V_{H1} comprises an amino acid sequence of SEQ ID NO: 42, and wherein the VL1 comprises an amino acid sequence of SEQ ID NO: 44.

In some embodiments, the second ABD binds specifically human NKp46 and comprises:
(i) a V_{H2} comprising a CDR-H1, 2 and 3 corresponding to:
the amino acid sequences of SEQ ID NO: 13, 14, and 15 respectively;
the amino acid sequences of SEQ ID NO: 16, 17, and 18 respectively;
the amino acid sequences of SEQ ID NO: 19, 20, and 21 respectively;
the amino acid sequences of SEQ ID NO: 22, 23, and 24 respectively; or
the amino acid sequences of SEQ ID NO: 16, 25 and 26 respectively; and
(ii) a V_{L2} comprising a CDR-L1, 2 and 3 corresponding to:
the amino acid sequences of SEQ ID NO: 27, 28, and 29 respectively;
the amino acid sequences of SEQ ID NO: 30, 31, and 32 respectively;
the amino acid sequences of SEQ ID NO: 33, 34, and 35 respectively;
the amino acid sequences of SEQ ID NO: 36, 37, and 38 respectively; or
the amino acid sequences SEQ ID NO: 39, 31 and 40 respectively.

In some embodiments, a) the V_{H2} comprises an amino acid sequence that is at least about 90% identical to the amino acid sequence of SEQ ID NO: 45, and wherein the V_{L1} comprises an amino acid sequence that is at least about 90% identical to the amino acid sequence of SEQ ID NO: 53; b) the V_{H2} comprises an amino acid sequence that is at least about 90% identical to the amino acid sequence of SEQ ID NO: 46, and wherein the V_{L2} comprises an amino acid sequence that is at least about 90% identical to the amino acid sequence of SEQ ID NO: 54; c) the V_{H2} comprises an amino acid sequence that is at least about 90% identical to the amino acid sequence of SEQ ID NO: 47, and wherein the V_{L2} comprises an amino acid sequence that is at least about 90% identical to the amino acid sequence of SEQ ID NO: 55; d) the V_{H2} comprises an amino acid sequence that is at least about 90% identical to the amino acid sequence of SEQ ID NO: 48, and wherein the V_{L2} comprises an amino acid sequence that is at least about 90% identical to the amino acid sequence of SEQ ID NO: 56; e) the V_{H2} comprises an amino acid sequence that is at least about 90% identical to the amino acid sequence of SEQ ID NO: 49, and wherein the V_{L2} comprises an amino acid sequence that is at least about 90% identical to the amino acid sequence of SEQ ID NO: 57; f) the V_{H2} comprises an amino acid sequence that is at least about 90% identical to the amino acid sequence of SEQ ID NO: 50, and wherein the V_{L2} comprises an amino acid sequence that is at least about 90% identical to the amino acid sequence of SEQ ID NO: 58; g) the V_{H2} comprises an amino acid sequence that is at least about 90% identical to the amino acid sequence of SEQ ID NO: 51, and wherein the V_{L2} comprises an amino acid sequence that is at least about 90% identical to the amino acid sequence of SEQ ID NO: 59; or h) the V_{H2} comprises an amino acid sequence that is at least about 90% identical to the amino acid sequence of SEQ ID NO: 52, and wherein the V_{L2} comprises an amino acid sequence that is at least about 90% identical to the amino acid sequence of SEQ ID NO: 60.

In some embodiments, a) the V_{H2} comprises an amino acid sequence of SEQ ID NO: 45, and wherein the V_{L1} comprises an amino acid sequence of SEQ ID NO: 53; b) the V_{H2} comprises an amino acid sequence of SEQ ID NO: 46, and wherein the V_{L2} comprises an amino acid sequence of SEQ ID NO: 54; c) the V_{H2} comprises an amino acid sequence of SEQ ID NO: 47, and wherein the V_{L2} comprises an amino acid sequence of SEQ ID NO: 55; d) the V_{H2} comprises an amino acid sequence of SEQ ID NO: 48, and wherein the V_{L2} comprises an amino acid sequence of SEQ ID NO: 56; e) the V_{H2} comprises an amino acid sequence of SEQ ID NO: 49, and wherein the V_{L2} comprises an amino acid sequence of SEQ ID NO: 57; f) the V_{H2} comprises an amino acid sequence of SEQ ID NO: 50, and wherein the V_{L2} comprises an amino acid sequence of SEQ ID NO: 58; g) the V_{H2} comprises an amino acid sequence of SEQ ID NO: 51, and wherein the V_{L2} comprises an amino acid sequence of SEQ ID NO: 59; or h) the V_{H2} comprises an amino acid sequence of SEQ ID NO: 52, and wherein the V_{L2} comprises an amino acid sequence of SEQ ID NO: 60.

In some embodiments, the binding protein comprises three polypeptide chains (I), (II) and (III) that form two ABDs, as defined below:

V_{1A}-C_{1A}-Hinge₁-(C_{H}2-C_{H}3)_{A} (I)

V_{1B}-C_{1B}-Hinge₂-(C_{H}2-C_{H}3)_{B}-L₁-V_{2A}-C_{2A}-Hinge₃ (II)

V_{2B}-C_{2B} (III)

wherein:
V_{1A} and V_{1B} form a binding pair V₁ (V_{H1}/V_{L1});
V_{2A} and V_{2B} form a binding pair V₂ (V_{H2}/V_{L2});
C_{1A} and C_{1B} form a pair C₁ (C_{H}1/C_{L}) and C_{2A} and C_{2B} form a pair C₂ (C_{H}1/C_{L}) wherein C_{H}1 is an immunoglobulin heavy chain constant domain 1 and C_{L} is an immunoglobulin light chain constant domain;
   Hinge₁, Hinge₂ and Hinge₃ are identical or different and correspond to all or part of an immunoglobulin hinge region;
   (C_{H}2-C_{H}3)_{A} and (C_{H}2-C_{H}3)_{B} are identical or different, and comprise an immunoglobulin heavy chain constant domain 2 (C_{H}2) and an immunoglobulin heavy chain constant domain 3 (C_{H}3); and
   L₁ is an amino acid linker.

In some embodiments, C_{1B} is an immunoglobulin heavy chain constant domain 1 (C_{H}1); C_{2A} is an immunoglobulin heavy chain constant domain 1 (C_{H}1); C_{L} corresponds to an immunoglobulin kappa light chain constant domain (C_{κ}); (C_{H}2-C_{H}3)_{A} corresponds to the amino acid sequence of SEQ ID NO: 69; (C_{H}2-C_{H}3)_{B} corresponds to the amino acid sequence of SEQ ID NO: 70; Hinge₁ corresponds to the amino acid sequence of SEQ ID NO:74; Hinge₂ corresponds to the amino acid sequence of SEQ ID NO:75; Hinge₃ corresponds to the amino acid sequence of SEQ ID NO: 77; and L₁ corresponds to the amino acid sequence of SEQ ID NO: 76.

In some embodiments, the method comprises at least two polypeptide chains linked by at least one disulfide bridge. In some embodiments, the polypeptide chains (I) and (II) are linked by at least one disulfide bridge between C_{1A} and Hinge₂ and/or wherein the polypeptide chains (II) and (III) are linked by at least one disulfide bridge between Hinge₃ and C_{2B}.

In some embodiments, V_{1A} is V_{L1} and V_{1B} is V_{H1}. In some embodiments, V_{2A} is V_{H2} and V_{2B} is V_{L2}.

In some embodiments, a) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 13; a CDR-H2 comprising the amino acid sequence of SEQ ID NO: 14; a CDR-H3 comprising the amino acid sequence of SEQ ID NO: 15; V_{L2} comprises a CDR-L1 comprising the amino acid sequence of SEQ ID NO: 27; a CDR-L2 comprising the amino acid sequence of SEQ ID NO: 28; a CDR-L3 comprising the amino acid sequence of SEQ ID NO: 29; b) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 16; a CDR-H2 comprising the amino acid sequence of SEQ ID NO: 17; a CDR-H3 comprising the amino acid sequence of SEQ ID NO: 18; V_{L2} comprises a CDR-L1 comprising the amino acid sequence of SEQ ID NO: 30; a CDR-L2 comprising the amino acid sequence of SEQ ID NO: 31; a CDR-L3 comprising the amino acid sequence of SEQ ID NO: 32; c) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 19; a CDR-H2 comprising the amino acid sequence of SEQ ID NO: 20; a CDR-H3 comprising the amino acid sequence of SEQ ID NO: 21; V_{L2} comprises a CDR-L1 comprising the amino acid sequence of SEQ ID NO: 33; a CDR-L2 comprising the amino acid sequence of SEQ ID NO: 34; a CDR-L3 comprising the amino acid sequence of SEQ ID NO: 35; d) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 22; a CDR-H2 comprising the amino acid sequence of SEQ ID NO: 23; a CDR-H3 comprising the amino acid sequence of SEQ ID NO: 24; V_{L2} comprises a CDR-L1 comprising the amino acid sequence of SEQ ID NO: 36; a CDR-L2 comprising the amino acid sequence of SEQ ID NO: 37; a CDR-L3 comprising the amino acid sequence of SEQ ID NO: 38; c) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 16; a CDR-H2 comprising the amino acid sequence of SEQ ID NO: 25; a CDR-H3 comprising the amino acid sequence of SEQ ID NO: 26; V_{L2} comprises a CDR-L1 comprising the amino acid sequence of SEQ ID NO: 39; a CDR-L2 comprising the amino acid sequence of SEQ ID NO: 31; a CDR-L3 comprising the amino acid sequence of SEQ ID NO: 40; f) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 13; a CDR-H2 comprising the amino acid sequence of SEQ ID NO: 14; a CDR-H3 comprising the amino acid sequence of SEQ ID NO: 15; V_{L2} comprises a CDR-L1 comprising the amino acid sequence of SEQ ID NO: 27; a CDR-L2 comprising the amino acid sequence of SEQ ID NO: 28; a CDR-L3 comprising the amino acid sequence of SEQ ID NO: 29; g) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 16; a CDR-H2 comprising the amino acid sequence of SEQ ID NO: 17; a CDR-H3 comprising the amino acid sequence of SEQ ID NO: 18; V_{L2} comprises a CDR-L1 comprising the amino acid sequence of SEQ ID NO: 30; a CDR-L2 comprising the amino acid sequence of SEQ ID NO: 31; a CDR-L3 comprising the amino acid sequence of SEQ ID NO: 32; h) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 19; a CDR-H2 comprising the amino acid sequence of SEQ ID NO: 20; a CDR-H3 comprising the amino acid sequence of SEQ ID NO: 21; V_{L2} comprises a CDR-L1 comprising the amino acid sequence of SEQ ID NO: 33; a CDR-L2 comprising the amino acid sequence of SEQ ID NO: 34; a CDR-L3 comprising the amino acid sequence of SEQ ID NO: 35; i) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 22; a CDR-H2 comprising the amino acid sequence of SEQ ID NO: 23; a CDR-H3 comprising the amino acid sequence of SEQ ID NO: 24; V_{L2} comprises a CDR-L1 comprising the amino acid sequence of SEQ ID NO: 36; a CDR-L2 comprising the amino acid sequence of SEQ ID NO: 37; a CDR-L3 comprising the amino acid sequence of SEQ ID NO: 38; or j) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 16; a CDR-H2 comprising the amino acid sequence of SEQ ID NO: 25; a CDR-H3 comprising the amino acid sequence of SEQ ID NO: 26; V_{L2} comprises a CDR-L1 comprising the amino acid sequence of SEQ ID NO: 39; a CDR-L2 comprising the amino acid sequence of SEQ ID NO: 31; a CDR-L3 comprising the amino acid sequence of SEQ ID NO: 40.

In some embodiments, (a) V_{H1} and V_{L1} corresponds to the amino acid sequences of SEQ ID NO: 41 and 43 respectively or corresponds to the amino acid sequences of SEQ ID NO: 42 and 44 respectively; and/or
(b) V_{H2} and V_{L2} corresponds to
   the amino acid sequences of SEQ ID NO: 45 and 53 respectively;
   the amino acid sequences of SEQ ID NO: 46 and 54 respectively;
   the amino acid sequences of SEQ ID NO: 47 and 55 respectively;
   the amino acid sequences of SEQ ID NO: 48 and 56 respectively;
   the amino acid sequences of SEQ ID NO: 49 and 57 respectively;
   the amino acid sequences of SEQ ID NO: 50 and 58 respectively;
   the amino acid sequences of SEQ ID NO: 51 and 59 respectively; or
   the amino acid sequences of SEQ ID NO: 52 and 60 respectively.

In some embodiments, V_{H1} comprises the amino acid sequence of SEQ ID NO: 41; V_{L1} comprises the amino acid sequence of SEQ ID NO: 43; V_{H2} comprises the amino acid sequence of SEQ ID NO: 45; V_{L2} comprises the amino acid sequence of SEQ ID NO: 53; V_{H1} comprises the amino acid sequence of SEQ ID NO: 41; V_{L1} comprises the amino acid sequence of SEQ ID NO: 43; V_{H2} comprises the amino acid sequence of SEQ ID NO: 46; V_{L2} comprises the amino acid sequence of SEQ ID NO: 54; V_{H1} comprises the amino acid sequence of SEQ ID NO: 41; V_{L1} comprises the amino acid sequence of SEQ ID NO: 43; VH2 comprises the amino acid sequence of SEQ ID NO: 47; V_{L2} comprises the amino acid sequence of SEQ ID NO: 55; V_{H1} comprises the amino acid sequence of SEQ ID NO: 41; V_{L1} comprises the amino acid sequence of SEQ ID NO: 43; V_{H2} comprises the amino acid sequence of SEQ ID NO: 48; V_{L2} comprises the amino acid sequence of SEQ ID NO: 56; V_{H1} comprises the amino acid sequence of SEQ ID NO: 41; V_{L1} comprises the amino acid sequence of SEQ ID NO: 43; V_{H2} comprises the amino acid sequence of SEQ ID NO: 49; VL₂ comprises the amino acid sequence of SEQ ID NO: 57; V_{H1} comprises the amino acid sequence of SEQ ID NO: 41; V_{L1} comprises the amino acid sequence of SEQ ID NO: 43; V_{H2} comprises the amino acid sequence of SEQ ID NO: 50; V_{L2} comprises the amino acid sequence of SEQ ID NO: 58; V_{H1} comprises the amino acid sequence of SEQ ID NO: 41; V_{L1} comprises the amino acid sequence of SEQ ID NO: 43; V_{H2} comprises the amino acid sequence of SEQ ID NO: 51; V_{L2} comprises the amino acid sequence of SEQ ID NO: 59; V_{H1} comprises the amino acid sequence of SEQ ID NO: 41; V_{L1} comprises the amino acid sequence of SEQ ID NO: 43; V_{H2} comprises the amino acid sequence of SEQ ID NO: 52; VL₂ comprises the amino acid sequence of SEQ ID NO: 60; V_{H1} comprises the amino acid sequence of SEQ ID NO: 42; V_{L1} comprises the amino acid sequence of SEQ ID NO: 44; V_{H2} comprises the amino acid sequence of SEQ ID NO: 45; VL₂ comprises the amino acid sequence of SEQ ID NO: 53; V_{H1} comprises the amino acid sequence of SEQ ID NO: 42; V_{L1} comprises the amino acid sequence of SEQ ID NO: 44; V_{H2} comprises the amino acid sequence of SEQ ID NO: 46; V_{L2} comprises the amino acid sequence of SEQ ID NO: 54; V_{H1} comprises the amino acid sequence of SEQ ID NO: 42; V_{L1} comprises the amino acid sequence of SEQ ID NO: 44; V_{H2} comprises the amino acid sequence of SEQ ID NO: 47; V_{L2} comprises the amino acid sequence of SEQ ID NO: 55; V_{H1} comprises the amino acid sequence of SEQ ID NO: 42; V_{L1} comprises the amino acid sequence of SEQ ID NO: 44; V_{H2} comprises the amino acid sequence of SEQ ID NO: 48; V_{L2} comprises the amino acid sequence of SEQ ID NO: 56; V_{H1} comprises the amino acid sequence of SEQ ID NO: 42; V_{L1} comprises the amino acid sequence of SEQ ID NO: 44; V_{H2} comprises the amino acid sequence of SEQ ID NO: 49; V_{L2} comprises the amino acid sequence of SEQ ID NO: 57; V_{H1} comprises the amino acid sequence of SEQ ID NO: 42; V_{L1} comprises the amino acid sequence of SEQ ID NO: 44; V_{H2} comprises the amino acid sequence of SEQ ID NO: 50; V_{L2} comprises the amino acid sequence of SEQ ID NO: 58; V_{H1} comprises the amino acid sequence of SEQ ID NO: 42; V_{L1} comprises the amino acid sequence of SEQ ID NO: 44; V_{H2} comprises the amino acid sequence of SEQ ID NO: 51; V_{L2} comprises the amino acid sequence of SEQ ID NO: 59; or V_{H1} comprises the amino acid sequence of SEQ ID NO: 42; V_{L1} comprises the amino acid sequence of SEQ ID NO: 44; V_{H2} comprises the amino acid sequence of SEQ ID NO: 52; V_{L2} comprises the amino acid sequence of SEQ ID NO: 60.

In some embodiments, (i) polypeptide (I) consists of an amino acid sequence of SEQ ID NO: 64; (ii) polypeptide (II) consists of an amino acid sequence of SEQ ID NO: 65; and (iii) polypeptide (III) consists of an amino acid sequence of SEQ ID NO: 66.

In some embodiments, the BCL-2 inhibitor is venetoclax, oblimersen (e.g., a sodium salt of oblimersen, also known as oblimersen sodium), obatoclax mesylate, palcitoclax, or LP-118. In some embodiments, the BCL-2 inhibitor is venetoclax or a pharmaceutically acceptable salt thereof.

In some embodiments, the DNA hypomethylating agent is a 5-azacytidine, a cytidine, a decitabine, or a guadecitabine. In some embodiments, the DNA hypomethylating agent is 5-azacytidine or a pharmaceutically acceptable salt thereof.

In some embodiments, the method comprises administering to the subject an effective amount of a combination comprising: (i) a binding protein comprising a first antigen binding domain (ABD) with binding specificity to CD123 and a second (ABD) with binding specificity to NKp46; (ii) a BCL-2 inhibitor; and (iii) a DNA hypomethylating agent.

In some embodiments, the leukemia is acute myeloid leukemia (AML). In some embodiments, the AML is relapsed or refractory. In some embodiments, the AML CD123 positive. In some embodiments, the AML is newly diagnosed AML, wherein the patient is ineligible for intensive chemotherapy. In some embodiments, the AML is CD123 positive, newly diagnosed AML and wherein the subject is ineligible for intensive chemotherapy. In some embodiments, the subject has a confirmed diagnosis of AML with positive CD123 tumor expression and ineligible for intensive chemotherapy. In some embodiments, the leukemia is B cell acute lymphoblastic leukemia (B-ALL). In some embodiments, the myelodysplastic syndrome is a high-risk myelodysplastic syndrome (HR-MDS). In some embodiments, the hematological disease or disorder is a myelodysplastic syndrome.

In some embodiments, the combination results in an increase in target cell lysis in the subject relative to administering any one of the binding protein, the BCL-2 inhibitor, or the DNA hypomethylating agent alone to the subject. In some embodiments, the combination results in a 1-fold, a 2-fold, a 3-fold, a 4-fold, a 5-fold, a 6-fold, or a 7-fold increase in target cell lysis in the subject relative to administering any one of the binding protein, the BCL-2 inhibitor, or the DNA hypomethylating agent alone to the subject.

In another aspect, provided herein is a method of treating or preventing a leukemia or a myelodysplastic syndrome in a subject in need thereof, said method comprising administering to the subject an effective amount of a combination comprising:
(i) a binding protein comprising a first ABD with binding specificity to CD123 and a second ABD with binding specificity to NKp46,
wherein the first ABD comprises a V_{H1} and a VL1, wherein:
   the VH1 comprises a complementary determining region (CDR)-H1, H2 and H3 corresponding to the amino acid sequences of SEQ ID NO: 1, 2, and 3; and
   the VL1 comprises a CDR-L1, L2 and L3 corresponding to the amino acid sequences of SEQ ID NO: 7, 8, and 9;
wherein the second antigen binding domain comprises a heavy chain variable domain (VH2) and a light chain variable domain (VL2), wherein:
   the VH2 comprises a CDR-H1, H2, and H3 corresponding to the amino acid sequences of SEQ ID NO: 13, 14, and 15;
   the V_{L2} comprises a CDR-L1, L2, and L3 corresponding to the amino acid sequences of SEQ ID NO: 27, 28, and 29; and
wherein all or part of the immunoglobulin Fc region or variant thereof binds to a human Fc-γ receptor;
(ii) a venetoclax or a pharmaceutically acceptable salt thereof; and
(iii) a 5-azacytidine or a pharmaceutically acceptable salt thereof,
   wherein the leukemia or a myelodysplastic syndrome is AML, B-ALL, or HR-MDS.

In another aspect, provided herein is a method of treating acute myeloid leukemia (AML) in a subject in need thereof, said method comprising administering to the subject an effective amount of a combination comprising:
(i) a binding protein comprising a first ABD with binding specificity to CD123 and a second ABD with binding specificity to NKp46,
wherein the first ABD comprises a V_{H1} and a VL1, wherein:
   the VH1 comprises a complementary determining region (CDR)-H1, H2 and H3 corresponding to the amino acid sequences of SEQ ID NO: 1, 2, and 3; and
   the VL1 comprises a CDR-L1, L2 and L3 corresponding to the amino acid sequences of SEQ ID NO: 7, 8, and 9;
wherein the second antigen binding domain comprises a heavy chain variable domain (VH2) and a light chain variable domain (VL2), wherein:
   the VH2 comprises a CDR-H1, H2, and H3 corresponding to the amino acid sequences of SEQ ID NO: 13, 14, and 15;
   the V_{L2} comprises a CDR-L1, L2, and L3 corresponding to the amino acid sequences of SEQ ID NO: 27, 28, and 29; and
wherein all or part of the immunoglobulin Fc region or variant thereof binds to a human Fc-γ receptor;
(ii) a venetoclax or a pharmaceutically acceptable salt thereof; and
(iii) a 5-azacytidine or a pharmaceutically acceptable salt thereof, wherein the AML is newly diagnosed AML, the subject has positive CD123 tumor expression, and wherein the subject is ineligible for intensive chemotherapy.

In yet another aspect, provided herein is a method of treating or preventing a leukemia or a myelodysplastic disorder in a subject in need thereof, said method comprising administering to the subject an effective amount of a combination comprising:
(i) a binding protein comprising a first antigen binding domain with binding specificity to CD123, a second antigen binding domain with binding specificity to NKp46, and all or part of an immunoglobulin Fc region or variant thereof that binds to a human Fc-γ receptor, wherein the binding protein comprises:
a polypeptide (I) that consists of an amino acid sequence of SEQ ID NO: 64;
a polypeptide (II) that consist of an amino acid sequence of SEQ ID NO: 65; and
a polypeptide (III) that consists of an amino acid sequence of SEQ ID NO: 66;
(ii) a venetoclax or a pharmaceutically acceptable salt thereof; and
(iii) a 5-azacytidine or a pharmaceutically acceptable salt thereof,
   wherein the leukemia or a myelodysplastic syndrome is AML, B-ALL, or HR-MDS.

In yet another aspect, provided herein is a method of treating acute myeloid leukemia (AML) in a subject in need thereof, said method comprising administering to the subject an effective amount of a combination comprising:
(i) a binding protein comprising a first antigen binding domain with binding specificity to CD123, a second antigen binding domain with binding specificity to NKp46, and all or part of an immunoglobulin Fc region or variant thereof that binds to a human Fc-γ receptor, wherein the binding protein comprises:
a polypeptide (I) that consists of an amino acid sequence of SEQ ID NO: 64;
a polypeptide (II) that consist of an amino acid sequence of SEQ ID NO: 65; and
a polypeptide (III) that consists of an amino acid sequence of SEQ ID NO: 66;
(ii) a venetoclax or a pharmaceutically acceptable salt thereof; and
(iii) a 5-azacytidine or a pharmaceutically acceptable salt thereof,
   wherein the AML is newly diagnosed AML, the subject has positive CD123 tumor expression, and wherein the subject is ineligible for intensive chemotherapy.

In some embodiments, the binding protein is administered to the subject intravenously, subcutaneously, intraperitoneally, or intramuscularly. In some embodiments, the binding protein is administered to the subject intravenously. In some embodiments, the binding protein is administered to the subject intravenously weekly for induction followed by every 4 weeks for maintenance.

In some embodiments, the binding protein, the BCL-2 inhibitor, and the DNA hypomethylating agent are administered simultaneously. In some embodiments, the binding protein, the BCL-2 inhibitor, and the DNA hypomethylating agent are administered sequentially. In some embodiments, the binding protein is administered to a subject for at least one cycle. In some embodiments, the binding protein is administered to a subject for at least two cycles. In some embodiments, the binding protein is administered to a subject for three or more cycles.

In one aspect, provided herein is an effective amount of a combination comprising:
(i) a binding protein comprising a first antigen binding domain (ABD) with binding specificity to CD123 and a second (ABD) with binding specificity to NKp46; and one or both of:
(ii) a BCL-2 inhibitor, and
(iii) a DNA hypomethylating agent
for use in a method for treating or preventing a leukemia or a myelodysplastic syndrome in a subject in need thereof.

In another aspect, provided herein is an effective amount of a combination comprising: (i) a binding protein comprising a first ABD with binding specificity to CD123 and a second ABD with binding specificity to NKp46,
wherein the first ABD comprises a VH1 and a VL1, wherein:
   the VH1 comprises a complementary determining region (CDR)-H1, H2 and H3 corresponding to the amino acid sequences of SEQ ID NO: 1, 2, and 3; and
   the VL1 comprises a CDR-L1, L2 and L3 corresponding to the amino acid sequences of SEQ ID NO: 7, 8, and 9;
wherein the second antigen binding domain comprises a heavy chain variable domain (VH2) and a light chain variable domain (VL2), wherein:
   the VH2 comprises a CDR-H1, H2, and H3 corresponding to the amino acid sequences of SEQ ID NO: 13, 14, and 15;
   the V_{L2} comprises a CDR-L1, L2, and L3 corresponding to the amino acid sequences of SEQ ID NO: 27, 28, and 29; and
wherein all or part of the immunoglobulin Fc region or variant thereof binds to a human Fc-γ receptor;
(ii) a venetoclax or a pharmaceutically acceptable salt thereof; and
(iii) a 5-azacytidine or a pharmaceutically acceptable salt thereof, for use in a method of treating or preventing an AML, a B-ALL, or a HR-MDS.

In yet another aspect, provided herein is an effective amount of a combination comprising: (i) a binding protein comprising a first antigen binding domain with binding specificity to CD123, a second antigen binding domain with binding specificity to NKp46, and all or part of an immunoglobulin Fc region or variant thereof that binds to a human Fc-γ receptor, wherein the binding protein comprises:
a polypeptide (I) that consists of an amino acid sequence of SEQ ID NO: 64;
a polypeptide (II) that consist of an amino acid sequence of SEQ ID NO: 65; and
a polypeptide (III) that consists of an amino acid sequence of SEQ ID NO: 66;
(ii) a venetoclax or a pharmaceutically acceptable salt thereof; and
(iii) a 5-azacytidine or a pharmaceutically acceptable salt thereof,
   for use in a method of treating or preventing an AML, a B-ALL, or a HR-MDS.

### BRIEF DESCRIPTION OF THE FIGURES

FIG. 1A-FIG. 1D report the results of a series of four independent replicates (FIG. 1A-replicate 1, FIG. 1B-replicate 2, FIG. 1C-replicate 3, FIG. 1D-replicate 4) of an in vitro cytotoxic assay on CD123-positive MOLM-13 cell line in the presence of 5-azacytidine (AZA) or Venetoclax (VEN). Percentage of live MOLM-13 cells after 48 h of treatment with AZA (grey circle) or VEN (black square) at concentrations ranging from 0.01 nM to 10 μM. Dashed line represents the target cell viability without any treatment after 48 h (cultured with complete culture medium).

FIG. 2A-FIG. 2F report the results of two series of three independent replicates of an in vitro cytotoxic assay against CD123-positive MOLM-13 cell line in the presence of AZA and VEN. Percentage of live MOLM-13 cells after 48 h of treatment with escalating concentrations of VEN (ranging from 0.01 nM to 10 μM) combined with 4 defined concentrations of AZA (0.01 μM, 0.1 μM, 1 μM, and 10 μM) (FIGS. 2A, 2C, and 2E). Percentage of live MOLM-13 cells after 48 h of treatment with escalating doses of AZA (ranging from 0.01 nM to 10 μM) combined with 4 defined concentrations of VEN (0.01 μM, 0.1 μM, 1 μM, and 10 μM) (FIGS. 2B, 2D, and 2F). Dashed lines represent the untreated target cell viability with complete culture medium or treated with a single agent in a defined concentration.

FIG. 3A-FIG. 3B show expression of CD123 on MOLM-13 cells in the presence of AZA or VEN. Median fluorescence intensity (MedFI) (FIG. 3A) and percentage (FIG. 3B) of CD123 expression on MOLM-13 cells after 48 h of treatment with escalating concentrations of AZA (grey circle) or VEN (black rectangle) ranging from 0.01 nM to 10 μM were displayed. Dashed lines represent the target cell CD123 expression in untreated control samples.

FIG. 4A-FIG. 4C report the results of in vitro killing assays on CD123-positive MOLM-13 cells in the presence of AZA or VEN. FIG. 4A reports the percentage of live MOLM-13 cells after 6 h, 24 h and 48 h of treatment with two defined concentrations of AZA or VEN (1 nM and 1 μM) without NK effector cells. FIG. 4B and FIG. 4C correspond to the same assay in the presence of NK effector cells from two independent healthy donors. In untreated condition, MOLM-13 cells were cultured alone or in the presence of NK effector cells in complete culture medium (white square). Dashed lines represent the MOLM-13 target cell viability at baseline (T0).

FIG. 5A-FIG. 5C show CD123 expression on MOLM-13 cells in the presence of AZA or VEN. FIG. 5A reports the MedFI of CD123 after 6 h, 24 h or 48 h of treatment at 1 nM and 1 μM without NK effector cells. FIG. 5B and FIG. 5C correspond to the same assay in the presence of NK effector cells from two independent healthy donors. In untreated condition, MOLM-13 cells were cultured alone or in the presence of NK effector cells in complete culture medium (white square). Dashed lines represent the MOLM-13 target cell viability at baseline (T0).

FIG. 6A-FIG. 6C show cell viability and phenotype characterization of MOLM-13 cells in the presence of AZA or VEN. FIG. 6A reports the percentage of live MOLM-13 cells after 3 (left) or 6 days (right) of treatment with defined concentrations of AZA (600 nM and 3 μM) or VEN (2 nM, 5 nM, and 10 nM). In untreated condition (no stim), MOLM-13 cells were cultured in complete culture medium (upper graphs). FIG. 6B reports the percentage of CD123 positive cells and in FIG. 6C reports the CD123 MedFI assessed in the same conditions. Dashed lines represent the target cell viability at baseline.

FIG. 7A-FIG. 7C report the results of in vitro cytotoxicity assays performed on NK cells isolated from three representative healthy donors (FIG. 7A-donor 1, FIG. 7B-donor 2, FIG. 7C-donor 3) in the presence of AZA or VEN. Percentage of live NK cells after 48 h of treatment with escalating concentrations of AZA (grey circle) or VEN (black square) ranging from 0.01 nM to 10 μM. Dashed lines represent the untreated NK cells control samples at baseline (TO) and after 2 days of culture (T1).

FIG. 8A-FIG. 8C show the expression of CD16a and NKp46 on NK cells measured by flow cytometry staining cocultured with MOLM-13 AML cells (ratio 1:1) in the presence of AZA or VEN. FIG. 8A shows the percentages of CD16a or NKp46-positive NK cells from a representative NK cell donor. FIG. 8B and FIG. 8C show CD16a and NKp46 expression measured by MedFI on NK cells from two independent healthy donors after treatment for 48 h with 1 μM of AZA or 1 nM of VEN.

FIG. 9 reports the results of an in vitro killing assay against CD123-positive MOLM-13 cells in the presence of increasing concentrations of CD123 NKCE using NK effector cells from one single donor. The number of live fluorescent MOLM-13 RFP cells after 64-72 h of treatment with 5, 10, or 100 ng/mL was measured. In untreated condition, MOLM-13 RFP cells were cultured alone or in the presence of NK effector cells. Target cell lysis was also evaluated in the presence of an isotype control at 100 ng/mL. The isotype control corresponds to a construct which does not bind CD123.

FIG. 10 reports the results of an in vitro killing assay against CD123-positive MOLM-13 cells in the presence of CD123 NKCE at 5 ng/ml, AZA at 600 nM, and VEN at 2 nM as single agents or in combination using NK effector cells from one healthy donor. The number of live fluorescent MOLM-13 RFP cells was measured after 64-72 h of stimulation with single or combined treatments. In untreated conditions, MOLM-13 RFP cells were cultured alone or in presence of NK effector cells from one healthy donor. Target cell lysis was also evaluated in the presence of an isotype control at 100 ng/ml. The isotype control corresponds to a construct which does not bind CD123.

FIG. 11 shows CD123 expression on MOLM-13 cells after 48 h of treatment with AZA or VEN. Flow cytometry plots show expression of CD123 on MOLM-13 cells in the presence of AZA or VEN. The percentage and MedFI of CD123-positive MOLM-13 cells after 48 h of treatment at 1 nM (concentration showing low killing potential) and 1 μM (showing enhanced cytotoxic effect) as compared to untreated MOLM-13 cells were measured. CD123-positive MOLM-13 cells have been identified after the exclusion of doublet cells and the selection of live cells (negative to Propidium Iodide staining).

FIG. 12 reports the results of an in vitro killing assay against CD123-positive MOLM-13 cells in the presence of increasing concentrations of CD123 NKCE. The number of live fluorescent MOLM-13 RFP cells after 64-72 h of treatment with CD123 NKCE at 5, 10, or 100 ng/mL was measured. In untreated condition, MOLM-13 RFP cells were cultured alone or in the presence of NK effector cells. Target cell lysis was also evaluated in the presence of an isotype control at 100 ng/ml.

FIG. 13A-13B report the results of an in vitro killing assay against CD123-positive MOLM-13 cells in the presence of increasing concentrations of CD123 NKCE and NK effector cells from two independent healthy donors (FIG. 13A—NK donor NK208, FIG. 13B—NK donor NK892). The number of live fluorescent MOLM-13 RFP cells after 64-72 h of treatment with CD123 NKCE at 5, 10, or 100 ng/mL was measured. In untreated condition, MOLM-13 RFP cells were cultured alone or in the presence of NK effector cells. Target cell lysis was also evaluated in the presence of an isotype control at 100 ng/ml.

FIG. 14A-14D report the results of in vitro killing assays against CD123-positive MOLM-13 cells in the presence of CD123 NKCE, AZA, and VEN as single agents or in combination and NK effector cells from four independently healthy donors (FIG. 14A—NK donor NK779, FIG. 14B—NK donor NK892, FIG. 14C—NK donor NK064, FIG. 14D—NK donor NK208). The number of live fluorescent MOLM-13 RFP cells after 64-72 h of treatment with CD123 NKCE at 5 ng/ml, AZA at 600 nM and/or VEN at 2 nM was measured. In untreated condition, MOLM-13 RFP cells were cultured alone or in the presence of NK effector cells. Target cell lysis was also evaluated in the presence of an isotype control at 100 ng/mL.

FIG. 15 reports an in vitro killing assay against CD123-positive MOLM-13 cells in the presence of CD123 NKCE at 5 ng/ml, AZA at 600 nM, and VEN at 2 nM as single agents or in combination. The graph shows the Area Under the Curve (AUC), averaged on replicates, by agent and healthy donor, corresponding to the tumor cell proliferation/cytotoxicity curves of MOLM-13 RFP cells.

FIG. 16A-16D report in vitro killing assays against CD123-positive MOLM-13 cells in the presence of CD123 NKE at 100 ng/mL, AZA at 600 nM, and/or VEN at 2 nM as single agents or in combination using the NK effector cells from four independent healthy donors (FIG. 16A—NK donor NK779, FIG. 16B—NK donor NK064, FIG. 16C—NK donor NK208, FIG. 16D-NK donor NK892). Data represent tumor cell proliferation calculated by counting the number of live fluorescent MOLM-13 RFP cells normalized to cell number at time 0 and expressed as a percentage, after treatment up to 64-72 h with single and combined treatment. In untreated condition, MOLM-13 RFP cells were cultured alone or in the presence of NK cells. Target cell lysis was also evaluated in the presence of an isotype control at 100 ng/mL which corresponds to a construct which does not bind CD123.

### DETAILED DESCRIPTION

This disclosure provides methods for treating or preventing a leukemia (e.g., AML) or a myelodysplastic syndrome in a subject in need thereof, said method comprising administering to the subject an effective amount of a combination comprising: (i) a multifunctional binding protein comprising a first antigen binding domain (ABD) with binding specificity to CD123 (an antigen of interest on tumoral target cells) and a second (ABD) with binding specificity to NKp46 (a surface biomarker on immune NK cells); and one or both of: (ii) a BCL-2 inhibitor, and (iii) a DNA hypomethylating agent.

### I. Definitions

That the disclosure may be more readily understood, select terms are defined below.

The articles “a” and “an” are used herein to refer to one or to more than one (i.e., to at least one) of the grammatical object of the article.

“About” or “approximately” as used herein when referring to a measurable value such as an amount, a temporal duration, and the like, is meant to encompass variations of ±20% or ±10%, ±5%, ±1%, or ±0.1% of a given value or range, as such variations are appropriate to perform the disclosed methods.

As used herein, the term “Cluster of Differentiation 123” or “CD123” marker is also known as “Interleukin 3 receptor alpha (IL3RA)” or “IL3R”, “IL3RX”, “IL3RY”, “IL3RAY”, “hIL-3Ra” and denotes an interleukin 3 specific subunit of a heterodimeric cytokine receptor. The functional interleukin 3 receptor is a heterodimer that comprises a specific alpha chain (IL-3A; CD123) and the IL-3 receptor beta chain (βθ; CD131) that is shared with the receptors for granulocyte macrophage colony stimulating factor (GM-CSF) and interleukin 5 (IL-5). CD123 is a type I integral transmembrane protein with a deduced Molecular Weight of about 43 kDa containing an extracellular domain involved in IL-3 binding, a transmembrane domain and a short cytoplasmic tail of about 50 amino acids. The extracellular domain is composed of two regions: a N-terminal region of about 100 amino acids, the sequence of which exhibits similarity to equivalent regions of the GM-CSF and IL-5 receptor alpha-chains; and a region proximal to the transmembrane domain that contains four conserved cysteine residues (SEQ ID NO: 117) and a motif, common to other members of this cytokine receptor family. The IL-3 binding domain comprises about 200 amino acid residue cytokine receptor motifs (CRMs) made up of two Ig-like folding domains. The extracellular domain of CD123 is highly glycosylated, with N-glycosylation necessary for both ligand binding and receptor signaling. The protein family gathers three members: IL3RA (CD123A), CSF2RA and IL5RA. The overall structure is well conserved between the three members, but sequence homologies are very low. One 300 amino-acid long isoform of CD123 has been discovered so far, but only on the RNA level which is accessible on the Getentry database under the accession number ACM241 16.1. A reference sequence of full-length human CD123 protein, including signal peptide, is available from the NCBI database under the accession number NP_002174.1 and under the Uniprot accession number P26951.

The extracellular domain of human CD123 (ECD) consists of the amino acid sequence of SEQ ID NO: 82. CD123 (the interleukin-3 receptor alpha chain IL-3Ra) is a tumor antigen overexpressed in a variety of hematological neoplasms. The majority of AML blasts express surface CD123 and this expression does not vary by subtype of AML. Higher expression of CD123 on AML at diagnosis has been reported to be associated with poorer prognosis. CD123 expression has been reported in other hematological malignancies including myelodysplasia, systemic mastocytosis, blastic plasmacytoid dendritic cell neoplasm (BPDCN), ALL and hairy cell leukemia.

As used herein, “Natural killer” or “NK cells” refers to a sub-population of lymphocytes that is involved in non-conventional immunity. NK cells can be identified by virtue of certain characteristics and biological properties, such as the expression of specific surface antigens including CD16, CD56 and/or CD57, NKp46 for human NK cells, the absence of the alpha/beta or gamma/delta TCR complex on the cell surface, the ability to bind to and kill cells that fail to express “self” MHC/HLA antigens by the activation of specific cytolytic machinery, the ability to kill tumor cells or other diseased cells that express a ligand for NK activating receptors, and the ability to release protein molecules called cytokines that stimulate or inhibit the immune response. Any of these characteristics and activities can be used to identify NK cells, using methods well known in the art. Any subpopulation of NK cells will also be encompassed by the term NK cells. Within the context herein “active” NK cells designate biologically active NK cells, including NK cells having the capacity of lysing target cells or enhancing the immune function of other cells. NK cells can be obtained by various techniques known in the art, such as isolation from blood samples, cytapheresis, tissue or cell collections, etc. Useful protocols for assays involving NK cells can be found in Natural Killer Cells Protocols (edited by Campbell K S and Colonna M). Human Press. pp. 219-238 (2000).

As used herein, the term “NKp46” marker, or “Natural cytotoxicity triggering receptor 1”, also known as “CD335” or “NKP46” or “NK-p46” or “LY94” refers to a protein or polypeptide encoded by the Ncr1 gene. A reference sequence of full-length human NKp46 protein is available from the NCBI database under the accession number NP_004820. The human NKp46 extracellular domain (ECD) corresponds to the amino acid sequence of SEQ ID NO: 80. The human NKp46 mRNA sequence is described in NCBI accession number NM_004829.

As used herein, the term “Fc-γ receptor” or “FcγR” or “Fc-gamma receptor” may refer to both activating and inhibitory FcγRs. Fc-gamma receptors (FcγR) are cellular receptors for the Fc region of an Immunoglobulin G (IgG). Upon binding of complexed IgG, FcγRs can modulate cellular immune effector functions, thereby linking the adaptive and innate immune systems, including ADCC-mediated immune responses. In humans, six classic FcγRs are currently reported: one high-affinity receptor (FcγRI) and five low-to-medium-affinity FcγRs (FcγRIIA, -B and -C, FcγRIIIA and -B). All FcγRs bind the same region on IgG Fc, yet with differing high (FcgRI) and low (FcgRII and FcgRIII) affinities. On a functional level, most of the FcγRs are activating receptors that can induce the cellular responses mentioned above, including ADCC-mediated immune response. Whereas FcγRI, FcγRIIa, FcγRIIc, and FcγRIIIa are activating receptors characterized by an intracellular immunoreceptor tyrosine-based activation motif (ITAM), FcγRIIb has an inhibition motif (ITIM) and is therefore inhibitory. Unless specified otherwise, the term FcγRs encompasses activating receptors, including FcγRI (CD64), FcγRIIA (CD32a), FcγRIIIa (CD16a) and FcγRIIIb (CD16b), and preferably FcγRIIIa (CD16a).

As used herein, the terms “FcγRIIIa (CD16a)” or “FcγRIIIa” or “CD16a” or “CD16” or “Cluster of Differentiation 16” may refer to a 50-65 kDa cell surface molecule expressed on mast cells, macrophages, and natural killer cells as a transmembrane receptor. FcγRIIIa is an activating receptor containing immunoreceptor tyrosine activating motifs (ITAMs) in the associated FcR γ-chain, ITAMs being necessary for receptor expression, surface assembly and signaling. CD16a is a low affinity receptor for IgG and is an important receptor mediating ADCC (antibody dependent cell mediated cytotoxicity) by NK cells. The high affinity receptor CD16a is preferentially found on NK cells and monocytes and induces antibody-dependent cellular cytotoxicity (ADCC) upon IgG binding.

As used herein, the terms “Format 5” or “F5”, “Format 25” or “F25”, “Format F6” or “F6” and “Format 26” or “F26” refer to specific binding protein configurations of bispecific or multispecific antibodies specifically designed to engineer multiple antigen binding domains into a single antibody molecule. The multifunctional binding proteins of the present disclosure which comprise a NKp46-binding domain and a CD123-binding domain are made based on the F25 format and are as used herein and interchangeably with the term “CD123 NKCE.” The F25 and format F26 respectively differ from format F5 and F6 in that one CH1/CL pair between the second and third polypeptide chain are swapped to form a CL/CH1 pair. One of ordinary skill in the art could envisage a “CD123 NKCE” in the F26 format. The F5 and F6 format have been previously described in the international patent application WO2017114694, incorporated herein by reference.

As used herein, the term “bispecific binding protein” refers to a binding protein that specifically binds to two different antigen targets (e.g., human NKp46 and human CD123) through at least two distinct antigen-binding domains (ABDs). A bispecific binding protein may be bivalent (two ABDs) or multivalent (more than two ABDs).

As used herein, the terms “specifically binds to” or “binds specifically to” refers to the ability of an antigen-binding domain (ABD) to bind to an antigen (e.g. human NKp46 and/or human CD123) containing an epitope with an Kd of at least about 1×10−6 M, 1×10−7 M, 1×10−8 M, 1×10−9 M, 1×10−10 M, 1×10−11 M, 1×10−12 M, or more, and/or to bind to an epitope with an affinity that is at least twofold greater than its affinity for a nonspecific antigen.

As used herein, the term “specifically binds to human NKp46 polypeptide” may refer to a specific binding toward a polypeptide comprising an amino acid sequence of SEQ ID NO: 80.

As used herein, the term “specifically to a human CD123 polypeptide” may refer to a specific binding toward a polypeptide comprising an amino acid sequence of SEQ ID NO: 82.

As used herein, the term “binds to a human Fc-γ receptor polypeptide” may refer to a binding toward a polypeptide comprising an amino acid sequence of SEQ ID NO: 83 or SEQ ID NO: 84.

Competitive binding assays and other methods for determining specific binding are further described below and are well known in the art. Expressions such as “specifically binds to”, or “with specificity for” are used interchangeably. Those terms are not construed to refer exclusively to those antibodies, polypeptides and/or multichain polypeptides which actually bind to the recited target/binding partner, but also to those which, although provided in a non-bound form, retain the specificity to the recited target. Binding specificity can be quantitatively determined by an affinity constant KA (or KA) and a dissociation constant KD (or KD).

As used herein, the term “affinity”, concentration (EC50) or the equilibrium dissociation constant (KD) means the strength of the binding of an antibody or polypeptide to an epitope. The affinity of an antibody is given by a specific type of equilibrium constant, which is the dissociation constant KD, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant KA is defined by 1/KD. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of surface plasmon resonance (SPR) screening (such as by analysis with a BIAcore™ SPR analytical device). In a non-limitative manner, a KD of less than 50 nM as determined by SPR, and under physiological conditions (e.g. at a pH ranging from 6 to 8 under normal buffer conditions), may generally be considered as indicative of specificity of binding for antigen-antigen binding domain (ABD) interactions.

As used herein, the term “and/or” is a grammatical conjunction that is to be interpreted as encompassing that one or more of the cases it connects may occur. For example, the wording “such native sequence proteins can be made using standard recombinant and/or synthetic methods” indicates that native sequence proteins can be made using standard recombinant and synthetic methods or native sequence proteins can be made using standard recombinant methods or native sequence proteins can be made using synthetic methods.

As used herein, the terms “therapeutically effective amount” of the multifunctional binding protein or pharmaceutical composition thereof is meant a sufficient amount of the antibody-like multifunctional binding protein to treat said cancer disease, at a reasonable benefit/risk ratio applicable to any medical treatment. It will be understood, however, that the total daily usage of the polypeptides and compositions of the present disclosure will be decided by the attending physician within the scope of sound medical judgment. The specific therapeutically effective dose level for any particular patient will depend upon a variety of factors including the disorder being treated and the severity of the disorder; activity of the specific polypeptide employed; the specific composition employed, the age, body weight, general health, sex and diet of the patient; the time of administration, route of administration, and rate of excretion of the specific polypeptide employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved.

As used herein, the term “subject” or “individual” or “patient” are used interchangeably and may encompass a human or a non-human mammal, rodent or non-rodent. The term includes, but is not limited to, mammals, e.g., humans including man, woman and child, other primates (monkey), pigs, rodents such as mice and rats, rabbits, guinea pigs, hamsters, cows, horses, cats, dogs, sheep and goats.

As used herein, the singular form “a”, “an” and “the” include plural references unless the context clearly dictates otherwise. For example, the term “a pharmaceutically acceptable carrier” encompasses a plurality of pharmaceutically acceptable carriers, including mixtures thereof.

As used herein, “a plurality of” may thus include two or two or more.

As used herein, “antibody” or “immunoglobulin” may refer to a natural or conventional antibody in which two heavy chains are linked to each other by disulfide bonds and each heavy chain is linked to a light chain by a disulfide bond. There are two types of light chain, lambda (λ) and kappa (κ). There are five main heavy chain classes (or isotypes) which determine the functional activity of an antibody molecule: IgM, IgD, IgG, IgA and IgE. Each chain contains distinct sequence domains. The light chain includes two domains or regions, a variable domain (VL) and a constant domain (CL). The heavy chain generally includes four domains, a variable domain (VH) and three constant domains (CH1, CH2 and CH3, collectively referred to as CH). In particular, classes IgG, IgA, and IgD have three heavy chain constant region domains, which are designated CH1 CH2, and CH3; and the IgM and IgE classes have four heavy chain constant region domains, CH1, CH2, CH3, and CH4. The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the antigen-binding fragment (Fab) of an immunoglobulin and consists of the variable portions of one light chain and one heavy chain.

As used herein, when referring to “IgG” or “Immunoglobulin G” in general, IgG1, IgG2, IgG3 and IgG4 are included, unless defined otherwise. In particular, IgG is IgG1. As used herein, the term “antibody-like” or “immunoglobulin-like” polypeptide may also refer to non-conventional or synthetic antigen-binding polypeptides or binding protein, including single domain antibodies and fragments thereof, in particular variable heavy chain of single domain antibodies, and chimeric, humanized, bispecific or multimeric antibodies.

As used herein, the term “domain” may be any region of a protein, generally defined on the basis of sequence homologies or identities, which is related to a specific structural or functional entity. Accordingly, the term “region”, as used in the context of the present disclosure, is broader in that it may comprise additional regions beyond the corresponding domain.

As used herein, the terms “linker region”, “linker peptide” or “linker polypeptide” or “amino acid linker” or “linker” refer to any amino acid sequence suitable for covalently linking two polypeptide domains, such as two antigen-binding domains together and/or a Fc region to one or more variable regions, such as one or more antigen-binding domains. Although the term is not limited to a particular size or polypeptide length, such amino acid linkers are generally less than 50 amino acids in length, preferably less than 30 amino acids in length, for instance 20 or less than 20 amino acids in length, for instance 15 or less than 15 amino acids in length. Such amino acid linkers may optionally comprise all or part of an immunoglobulin polypeptide chain, such as all or part of a hinge region of an immunoglobulin. Alternatively, the amino acid linker may comprise a polypeptide sequence that is not derived from a hinge region of an immunoglobulin, or even that is not derived from an immunoglobulin heavy or light polypeptide chain.

As used herein, an immunoglobulin hinge region, or a fragment thereof, may thus be considered as a particular type of linker, which is derived from an immunoglobulin polypeptide chain.

As used herein, the term “hinge region” or “hinge” refers to a generally flexible region and born by the corresponding heavy chain polypeptides, and which separates the Fc and Fab portions of certain isotypes of immunoglobulins, more particularly of the IgG, IgA or IgD isotypes. Such hinge regions are known in the Art to depend upon the isotype of immunoglobulin which is considered. For native IgG, IgA and IgD isotypes, the hinge region thus separates the CH1 domain and the CH2 domain and is generally cleaved upon papain digestion. On the other hand, the region corresponding to the hinge in IgM and IgE heavy chains is generally formed by an additional constant domain with lower flexibility. Additionally, the hinge region may comprise one or more cysteines involved in interchain disulfide bonds. The hinge region may also comprise one or more binding sites to a Fcγ receptor, in addition to FcγR binding sites born by the CH2 domain, when applicable. Additionally, the hinge region may comprise one or more post-translational modification, such as one or more glycosylated residues depending on the isotype which is considered. Thus, it will be readily understood that the reference to the term “hinge” throughout the specification is not limited to a particular set of hinge sequences or to a specific location on the structure. Unless instructed otherwise, the hinge regions which are still particularly considered comprise all or part of a hinge from an immunoglobulin belonging to one isotype selected from: the IgG isotype, the IgA isotype and the IgD isotype; in particular the IgG isotype.

As used herein, the terms “CH domain”, or “CH domain”, or “constant domain”, can be used interchangeably and refer to any one or more heavy chain immunoglobulin constant domain(s). Such CH domains are natively folded as immunoglobulin-like domains, although they may be partly disordered in an isolated form (e.g., CH1 domains when not associated with the constant domain of a light chain (CL)). Unless instructed otherwise, the term may thus refer to a CH1 domain, a CH2 domain, a CH3 domain; or any combinations thereof.

As used herein, the terms “CH1 domain”, or “CH1 domain”, or “constant domain 1”, can be used interchangeably and refer to the corresponding heavy chain immunoglobulin constant domain 1.

As used herein, the term “CH2 domain”, or “CH2 domain”, or “constant domain 2” can be used interchangeably and refer to the corresponding heavy chain immunoglobulin constant domain 2.

As used herein, the term “CH3 domain”, or “CH3 domain”, or “constant domain 3” can be used interchangeably and refer to the corresponding heavy chain immunoglobulin constant domain 3.

As used herein, the term “CH2-CH3”, as in (CH2-CH3) A and (CH2-CH3) B, thus refers to a polypeptide sequence comprising an immunoglobulin heavy chain constant domain 2 (CH2) and an immunoglobulin heavy chain constant domain 3 (CH3).

As used herein, the term “CL domain”, or “CL domain” can be used interchangeably and refer to the corresponding light chain immunoglobulin constant domain. Unless instructed otherwise, this term may thus encompass a CL domain of the kappa (κ or K) or lambda (λ) class of immunoglobulin light chains, including all known subtypes (e.g. λ1, λ2, λ3, and λ7). In particular, when the CL domain is of the kappa class, it may also be referred herein as a Cκ or CK or Ck domain.

As used herein, the terms “pair C (CH1/CL)”, or “paired C (CH1/CL)” “refers to one constant heavy chain domain 1 and one constant light chain domain (e.g., a kappa (κ or K) or lambda (λ) class of immunoglobulin light chains) bound to one another by covalent or non-covalent bonds, preferably non-covalent bonds; thus forming a heterodimer. Unless specified otherwise, when the constant chain domains forming the pair are not present on a same polypeptide chain, this term may thus encompass all possible combinations. Preferably, the corresponding CH1 and CL domains will thus be selected as complementary to each other, such that they form a stable pair C (CH1/CL).

Advantageously, when the binding protein comprises a plurality of paired C domains, such as one “pair C1 (CH1/CL)” and one “pair C2 (CH1/CL)”, each CH1 and CL domain forming the pairs will be selected so that they are formed between complementary CH1 and CL domains. Examples of complementary CH1 and CL domains have been previously described in the international patent applications WO2006064136 or WO2012089814 or WO2015197593A1.

Unless instructed otherwise, the terms “pair C1 (CH1/CL)” or “pair C2 (CH1/CL)” may refer to distinct constant pair domains (C1 and C2) formed by identical or distinct constant heavy 1 domains (CH1) and identical or distinct constant light chain domains (CL). Preferably, the terms “pair C1 (CH1/CL)” or “pair C2 (CH1/CL)” may refer to distinct constant pair domains (C1 and C2) formed by identical constant heavy 1 domains (CH1) and identical constant light chain domains (CL).

As used herein, the term “Fc region” or “fragment crystallizable region”, or alternatively “Fc portion”, encompasses all or parts of the “Fc domain”, which may thus include all or parts of an immunoglobulin hinge region (which natively bears a first binding site to FcγRs), a CH2 domain (which natively bears a second binding site to FcγRs), and a CH3 domain of an immunoglobulin (e.g. of an IgG, IgA or IgD immunoglobulin), and/or when applicable of a CH4 domain of an immunoglobulin (e.g. for IgM and IgE). Preferably, the Fc region includes all or parts of, at least, a CH2 domain and a CH3 domain, and optionally all or parts of an immunoglobulin hinge region. The term may thus refer to a molecule comprising the sequence of a non-antigen-binding fragment resulting from digestion of an antibody or produced by other means, whether in monomeric or multimeric form, and can contain the hinge region. The original immunoglobulin source of the native Fc is, in particular, of human origin and can be any of the immunoglobulins, although IgG1 are preferred. Native Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 13 depending on class (e.g., IgG, IgA, and IgE) or subclass (e.g., IgG1, IgG2, IgG3, IgGA1, and IgGA2). One example of a native Fc is a disulfide-bonded dimer resulting from papain digestion of an IgG. The term “native Fc” as used herein is generic to the monomeric, dimeric, and multimeric forms. Under that terminology, a “Fc region” may thus comprise or consist of CH2-CH3 (e.g., (CH2-CH3) A or (CH2-CH3) B or a binding pair thereof, and optionally all or part of an immunoglobulin hinge region, comprising a binding site to a human FcγR. Unless specified otherwise, the term “Fc region” may refer to either a native or variant Fc region.

The term “Fc variant” as used herein refers to a molecule or sequence that is modified from a native Fc but still comprises a binding site for the receptor, FcRn (neonatal Fc receptor). Exemplary Fc variants, and their interaction with the receptor, are known in the art. Thus, the term “Fc variant” can comprise a molecule or sequence that is humanized from a non-human native Fc. Furthermore, a native Fc comprises regions that can be removed because they provide structural features or biological activity that are not required for the antibody-like binding proteins of the invention. Thus, the term “Fc variant” comprises a molecule or sequence that lacks one or more native Fc sites or residues, or in which one or more Fc sites or residues has be modified, that affect or are involved in: (1) disulfide bond formation, (2) incompatibility with a selected host cell, (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

The fragment crystallizable (Fc) regions (e.g., native or variant) according to the present disclosure retain a capacity to bind to a human Fc-γ receptor polypeptide (Fcγ) which generally occurs on native Fc regions through binding of the antibody Fc-hinge region. As a reference, overall structures of IgG1, IgG2, and IgG4 are similar with more than 90% sequence homology, the major differences residing in the hinge region and CH2 domain, which form primary binding sites to FcγRs. The hinge region also functions as a flexible linker between the Fab and Fc portion.

Fc regions having one or more amino acid modifications (e.g., substitutions, deletions, insertions) in one or more portions, which modifications increase the affinity and avidity of the variant Fc region for an FcγR (including activating and inhibitory FcγRs) are further considered as Fc regions. In some embodiments, said one or more amino acid modifications increase the affinity of the Fc region for FcγRIIIA and/or FcγRIIA. In another embodiment, the variant Fc region further specifically binds FcγRIIB with a lower affinity than does the Fc region of the reference parent antibody (e.g., an antibody having the same amino acid sequence as the antibody except for the one or more amino acid modifications in the Fc region). Hence, native and variant Fc regions considered herein generally comprise a domain (i.e., a CH2 domain) capable of binding to human CD16, e.g., a human Fc domain comprising N-linked glycosylation at amino acid residue N297 (according to EU numbering).

As used herein, the term “Fc-competent” thus refers to a binding protein that is capable of binding specifically to a FcγR, in particular of an activating FcγR, in particular to one selected from FcγRI (CD64a), FcγRIIa (CD32a), and FcγRIIIa (CD16a), and more particularly to FcγRIIIa (CD16a).

Alternatively, several modifications are reported to directly affect the binding to FcγRs, including mutation on residues 297 (according to EU numbering), or alternatively on residues 234 and 235 in the lower hinge region (according to the EU numbering system). As used herein, the term “Fc-silent” refers to a binding protein with a Fc region, wherein the Fc region lacks a binding site to a FcγR (e.g., a Fc region lacking a CH2 domain with said binding site and hinge region with said binding site); in particular FcγRI, FcγRIIa, and FcγRIIIa, and more particularly to FcγRIIIa (CD16a).

As used herein, the term “variable”, as in “variable domain”, refers to certain portions of the relevant binding protein which differ extensively in sequence between and among antibodies and are used in the specific recognition and binding of a particular antibody for its particular target. However, the variability is not evenly distributed throughout the entire variable domains of antibodies. The variability is concentrated in three segments called complementarity determining regions (CDRs; i.e., CDR1, CDR2, and CDR3) also known as hypervariable regions, both in the light chain and the heavy chain variable domains. The more highly conserved portions of variable domains are called the framework (FR) regions or sequences.

As used herein, the term “VH domain,” or “VH domain” can be used interchangeably and refer to the corresponding heavy chain immunoglobulin variable domain.

As used herein, the term “VL domain”, or “VL domain” can be used interchangeably and refer to the corresponding light chain immunoglobulin variable domain.

When the VH or VL domains are associated to a first antigen-binding domain (ABD) or to a second antigen-binding domain, they may also be respectively referred herein as “VH1” and “VL1”, or “VH2” and “VL2”.

The terms “binding pair V (VH/VL)”, “VH/VL pair” or “(VH/VL) pair” or “VL/VH pair” or “(VL/VH) pair” can be used interchangeably. Heavy chain and light chain variable domain can pair in parallel to form the antigen binding domains (ABDs). Each binding pair includes both a VH and a VL region. Unless instructed otherwise, these terms do not specify which immunoglobulin variable regions are VH or VL regions and which ABD will bind specifically the protein expressed on the surface of an immune effector cell or a target cell (e.g.,).

As used herein, the term “hypervariable region’ when used herein refers to the amino acid residues of an antibody that are responsible for antigen binding. This term may be substituted by the terms “Complementarity Determining Regions” or “CDRs”.

Thus, as used herein “Complementarity Determining Regions” or “CDRs” refer to amino acid sequences that together define the binding affinity and specificity of the natural Fv region of a native immunoglobulin binding site. The light and heavy chains of an immunoglobulin each have three CDRs, designated CDR-L1, CDR-L2, CDR-L3 and CDR-H1, CDR-H2, CDR-H3, respectively. A conventional antibody antigen-binding domain, therefore, includes six CDRs, comprising the CDR set from each of a heavy and a light chain variable region. Also, as used herein, “Framework Regions” (FRs) refer to amino acid sequences interposed between CDRs, i.e., to those portions of immunoglobulin light and heavy chain variable regions that are relatively conserved among different immunoglobulins in a single species. The light and heavy chains of an immunoglobulin each have four FRs, designated FR-L1, FR-L2, FR-L3, FR-L4, and FR-H1, FR-H2, FR-H3, FR-H4, respectively. Accordingly, the light chain variable domain may thus be designated as (FR-L1)-(CDR-L1)-(FR-L2)-(CDR-L2)-(FR-L3)-(CDR-L3)-(FR-L4) and the heavy chain variable domain may thus be designated as (FR-H1)-(CDR-H1)-(FR-H2)-(CDR-H2)-(FR-H3)-(CDR-H)-(FR4-H3).

The hypervariable region generally comprises amino acid residues from a “complementarity-determining region” or “CDR” (e.g. residues 24-34 (L1), 50-56 (L2) and 89-97 (L3) in the light-chain variable domain and 31-35 (H1), 50-65 (H2) and 95-102 (H3) in the heavy-chain variable domain; Kabat et al. 1991) and/or those residues from a “hypervariable loop” (e.g. residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987; 196:901-917). The numbering of amino acid residues in this region is performed by the method described in Kabat et al., supra. Accordingly, phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

Optionally, CDRs are as defined by EU, Kabat, Chotia or IMGT numbering. Correspondences between those classifications are known in the Art, by reference to the IMGT®, or international ImMunoGeneTics information system® (CNRS and Montpellier University), and as further detailed in Lefranc (Biomolecules; 2014; 4, 1102-1139) and Dondelinger (Frontiers in Immunology; 2018; 9, 2278). CDRs may also be defined according to the Honegger-Pluckthun (“Honegger”) numbering scheme described in Honnegger and Pluckthun (2001), J. Mol. Biol., vol. 309 (3): 657-670.

Unless instructed otherwise, the numbering of residues will be considered herein by reference to the EU, Kabat, Chotia, IMGT, or Honegger-Pluckthun numbering convention. In case of conflict regarding the exact position of hypervariable regions within a reference sequence, the Kabat numbering convention will prevail. In case of conflict regarding the exact position of constant regions within a reference sequence, the EU numbering convention will prevail. Further,

As used herein, the term “cytotoxicity” refers to the quality of a compound, such as the multifunctional binding protein according to the present disclosure, to be toxic to tumoral cells as measured by target cell lysis. Cytotoxicity may be induced by different mechanisms of action and can thus be divided into cell-mediated cytotoxicity, apoptosis, antibody-dependent cell-mediated cytotoxicity (ADCC), antibody-dependent cellular phagocytosis (ADCP) or complement-dependent cytotoxicity (CDC). In some embodiments, the combination therapies as described herein results in an increase in target cell lysis in the subject relative to administering any one of the binding protein, the BCL-2 inhibitor, or the DNA hypomethylating agent alone to the subject. In some embodiments, the combination therapies as described herein result in a 1-fold, a 2-fold, a 3-fold, a 4-fold, a 5-fold, a 6-fold, or a 7-fold increase in target cell lysis in the subject relative to administering any one of the binding protein, the BCL-2 inhibitor, or the DNA hypomethylating agent alone to the subject.

As used herein, the term “antibody-dependent cell-mediated cytotoxicity” or “ADCC” refers to a mechanism of cell-mediated immune defence whereby an effector cell of the immune system actively lyses a target cell, whose membrane-surface antigens have been bound by specific antibodies or the multifunctional binding protein of the present disclosure.

As used herein, a “pharmaceutically acceptable carrier” is intended to include any and all carrier (such as any solvents, dispersion media, coatings, antibacterial and antifungal agents, isotonic and absorption delaying agents, and the like) which is compatible with pharmaceutical administration, in particular parenteral administration. The use of such media and agents for pharmaceutically active substances are known. Except insofar as any conventional media or agent is incompatible with the active compound, such media can be used in the compositions of the present disclosure. For example, preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In a non-exhaustive manner, pharmaceutically acceptable carriers include, but are not limited to, 0.01-0.1M (e.g., 0.05M) phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like. More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringeability exists. It should be stable under the conditions of manufacture and storage and will in an embodiment be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal and the like. In certain embodiments, isotonic agents are included, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

As used herein, and unless instructed otherwise, the term “at least one” may encompass “one or more,” or even “two or more” (or “a plurality”). For instance, it may encompass 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more than 100.

As used herein, and unless instructed otherwise, the term “less than” may encompass all values from 0 to the corresponding threshold, For instance, it may encompass less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or less than 100, when applicable.

As used herein, “treating” refers to a therapeutic use (i.e., on a subject having a given disease) and means reversing, alleviating, inhibiting the progress of one or more symptoms of such disorder or condition. Therefore, treatment does not only refer to a treatment that leads to a complete cure of the disease, but also to treatments that slow down the progression of the disease and/or prolong the survival of the subject.

As used herein, “preventing” means a prophylactic use (i.e., on a subject susceptible of developing a given disease and encompasses the treatment of relapsed AML patient).

II. Multifunctional Binding proteins

As used herein, a “binding protein of the present disclosure” refers to multifunctional binding proteins comprising a first and a second antigen binding domains (ABDs) and all or part of an immunoglobulin Fc region or variant thereof, wherein the first ABD binds specifically to human CD123 and the second ABD binds specifically to human NKp46 which can further comprise all or part of the immunoglobulin Fc region or variant thereof.

As used herein, the term “multifunctional binding protein” encompass a multi-chain protein, including but not limited to antibody-like polypeptide or protein formats, which comprises at least one first variable region (e.g. a first immunoglobulin heavy chain variable domain (VH) and/or an immunoglobulin light chain variable domain (VL)) binding specifically to a human CD123 polypeptide, and at least one second variable region (e.g. a second immunoglobulin heavy chain variable domain (VH) and/or immunoglobulin light chain variable domain (VL)) binding specifically to a human NKp46 polypeptide. Although not limited specifically to a particular type of construct, one general embodiment is particularly considered throughout the specification: the polypeptide constructs reported in WO2015197593 and WO2017114694, each of which is incorporated herein by reference. In particular, the multifunctional binding protein such as those reported in WO2015197593 and WO2017114694, may encompass any construct comprising one or more polypeptide chains.

In some embodiments, the binding protein is characterized in that it comprises a first antigen binding domain with binding specificity to CD123 and a second antigen binding domain with binding specificity to NKp46.

In certain embodiments, the binding proteins comprise:
an antigen binding domain which binds specifically to human CD123 with a KD of less than 10 nM, in particular with a KD of less than 0.5 nM, as determined by SPR, under physiological conditions;
an antigen binding domain which binds specifically to human NKp46 with a KD of less than 50 nM, in particular with a KD of less than 20 nM, as determined by SPR, under physiological conditions.

In some embodiments, the binding protein is a bispecific NK cell engager (NKCE) with a competent Fc domain that can bind to CD16a (FcγRIIIa). The NKCE of the disclosure functions as a trifunctional molecule that binds NKp46 and CD16a on the surface of the NK cells and CD123 on malignant cells. Co-engagement of a NK cell and a malignant cell by the CD123 NKCE of the present disclosure leads to the formation of an immunological synapse which induces NK-cell activation and degranulation.

In some embodiments, the NKCE is characterized in that it comprises a first antigen binding domain with binding specificity to CD123 and a second antigen binding domain with binding specificity to NKp46, wherein the first antigen binding domain comprises:
a heavy chain variable domain (VH1) comprising a CDR-H1, H2, and H3 corresponding to the amino acid sequences of SEQ ID NO: 1, 2, and 3, respectively or corresponding to the amino acid sequences of SEQ ID NO: 4, 5, and 5, respectively; and
a light chain variable domain (VL1) comprising a CDR-L1, L2, and L3 corresponding to the amino acid sequences of SEQ ID NO: 7, 8, and 9, respectively or corresponding to the amino acid sequences of SEQ ID NO: 10, 11, and 12, respectively.

In some embodiments, the binding protein is characterized in that the first antigen binding domain with binding specificity comprises:
a heavy chain variable domain (VH1) comprising a CDR-H1, H2, and H3 corresponding to the amino acid sequences of SEQ ID NO: 1, 2, and 3, respectively or corresponding to the amino acid sequences of SEQ ID NO: 4, 5, and 5, respectively; and
a light chain variable domain (VL1) comprising a CDR-L1, L2, and L3 corresponding to the amino acid sequences of SEQ ID NO: 7, 8, and 9, respectively or corresponding to the amino acid sequences of SEQ ID NO: 10, 11, and 12, respectively.

In some embodiments, the binding protein is characterized in that it comprises the V_{H1} comprises an amino acid sequence that is at least about 90% identical to the amino acid sequence of SEQ ID NO: 41, and wherein the V_{L1} comprises an amino acid sequence that is at least about 90% identical to the amino acid sequence of SEQ ID NO: 43; or the V_{H1} comprises an amino acid sequence that is at least about 90% identical to the amino acid sequence of SEQ ID NO: 42, and wherein the V_{L1} comprises an amino acid sequence that is at least about 90% identical to the amino acid sequence of SEQ ID NO: 44.

In some embodiments, the binding protein is characterized in that it comprises the VH1 comprises an amino acid sequence of SEQ ID NO: 41, and wherein the V_{L1} comprises an amino acid sequence of SEQ ID NO: 43; or the V_{H1} comprises an amino acid sequence of SEQ ID NO: 42, and wherein the V_{L1} comprises an amino acid sequence of SEQ ID NO: 44.

In some embodiments, the binding protein comprises a second antigen binding domain with binding specificity to NKp46. In some embodiments, the second antigen binding domain comprises CDRs defined by Kabat numbering. In some embodiments, the second antigen binding domain comprises CDRs defined by IMGT numbering. In some embodiments, the second antigen binding domain comprises CDRs defined by Chothia numbering. In some embodiments, the second antigen binding domain comprises CDRs defined by Honegger numbering. In some embodiments, the second antigen binding domain comprises CDRs as defined in Table A.

TABLE A
Example Anti-NKp46 CDRs

| | Kabat | IMGT | Chothia | Honegger |
|---|---|---|---|---|
| CDRL1 | RASQDISNYLN | QDISNY (SEQ | SQDISNY | ASQDISNY (SEQ |
| | (SEQ ID NO: 27) | ID NO: 99) | (SEQ ID NO: | ID NO: 111) |
| | | | 105) | |
| CDRL2 | YTSRLHS (SEQ ID | YTS (SEQ ID | YTS (SEQ | YTSRLHSGVPSR |
| | NO: 28) | NO: 100) | ID NO: 106) | (SEQ ID NO: 112) |
| CDRL3 | QQGNTRPWT (SEQ | QQGNTRPWTF | GNTRPW | GNTRPW (SEQ ID |
| | ID NO: 29) | (SEQ ID NO: 101) | (SEQ ID NO: | NO: 107) |
| | | or QQGNTRPWT | 107) | |
| | | (SEQ ID NO: 116) | | |
| CDRH1 | DYVIN (SEQ ID | GYTFSDYV | GYTFSDY | ASGYTFSDYV |
| | NO: 13) | (SEQ ID NO: 102) | (SEQ ID NO: | (SEQ ID NO: 113) |
| | | | 108) | |
| CDRH2 | EIYPGSGTNYYNE | IYPGSGTN (SEQ | PGSG (SEQ | IYPGSGTNYYNEK |
| | KFKA (SEQ ID NO: | ID NO: 103) | ID NO: 109) | FKAK (SEQ ID |
| | 14) | | | NO: 114) |
| CDRH3 | RGRYGLYAMDY | ARRGRYGLYA | GRYGLYA | RGRYGLYAMD |
| | (SEQ ID NO: 15) | MDY SEQ ID | MD (SEQ | (SEQ ID NO: 115) |
| | | NO: 104) | ID NO: 110) | |

In some embodiments, the binding protein is characterized in that the second antigen binding domain with binding specificity to NKp46 comprises:
a. a second heavy chain variable domain (VH2) comprising a CDR-H1, H2, and H3 corresponding to the amino acid sequences of:
i. SEQ ID NO: 13, 14, and 15, respectively;
ii. SEQ ID NO: 16, 17, and 18, respectively;
iii. SEQ ID NO: 19, 20, and 21, respectively;
iv. SEQ ID NO: 22, 23, and 24, respectively; or
v. SEQ ID NO: 16, 25, and 26, respectively; and
b. a second light chain variable domain (VL2) comprising a CDR-L1, L2, and L3 corresponding to the amino acid sequences of:
   i. SEQ ID NO: 27, 28, and 29, respectively;
   ii. SEQ ID NO: 30, 31, and 32, respectively;
   iii. SEQ ID NO: 33, 34, and 35, respectively;
   iv. SEQ ID NO: 36, 37, and 38, respectively; or
   v. SEQ ID NO: 39, 31, and 40, respectively.

In some embodiments, the binding protein is characterized in that:
a. the VH2 comprises an amino acid sequence that is at least about 90% identical to the amino acid sequence of SEQ ID NO: 45, and wherein the VL1 comprises an amino acid sequence that is at least about 90% identical to the amino acid sequence of SEQ ID NO: 53; or
b. the VH2 comprises an amino acid sequence that is at least about 90% identical to the amino acid sequence of SEQ ID NO: 46, and wherein the V_{L2} comprises an amino acid sequence that is at least about 90% identical to the amino acid sequence of SEQ ID NO: 54
c. the VH2 comprises an amino acid sequence that is at least about 90% identical to the amino acid sequence of SEQ ID NO: 47, and wherein the V_{L2} comprises an amino acid sequence that is at least about 90% identical to the amino acid sequence of SEQ ID NO: 55;
d. the VH2 comprises an amino acid sequence that is at least about 90% identical to the amino acid sequence of SEQ ID NO: 48, and wherein the V_{L2} comprises an amino acid sequence that is at least about 90% identical to the amino acid sequence of SEQ ID NO: 56;
e. the VH2 comprises an amino acid sequence that is at least about 90% identical to the amino acid sequence of SEQ ID NO: 49, and wherein the V_{L2} comprises an amino acid sequence that is at least about 90% identical to the amino acid sequence of SEQ ID NO: 57;
f. the VH2 comprises an amino acid sequence that is at least about 90% identical to the amino acid sequence of SEQ ID NO: 50, and wherein the V_{L2} comprises an amino acid sequence that is at least about 90% identical to the amino acid sequence of SEQ ID NO: 58;
g. the VH2 comprises an amino acid sequence that is at least about 90% identical to the amino acid sequence of SEQ ID NO: 51, and wherein the V_{L2} comprises an amino acid sequence that is at least about 90% identical to the amino acid sequence of SEQ ID NO: 59; or
h. the VH2 comprises an amino acid sequence that is at least about 90% identical to the amino acid sequence of SEQ ID NO: 52, and wherein the V_{L2} comprises an amino acid sequence that is at least about 90% identical to the amino acid sequence of SEQ ID NO: 60.

In some embodiments, the binding protein is characterized in that:
a. the VH2 comprises an amino acid sequence of SEQ ID NO: 45, and wherein the VL1 comprises an amino acid sequence of SEQ ID NO: 53; or
b. the VH2 comprises an amino acid sequence of SEQ ID NO: 46, and wherein the V_{L2} comprises an amino acid sequence of SEQ ID NO: 54
c. the VH2 comprises an amino acid sequence of SEQ ID NO: 47, and wherein the V_{L2} comprises an amino acid sequence of SEQ ID NO: 55;
d. the VH2 comprises an amino acid sequence of SEQ ID NO: 48, and wherein the V_{L2} comprises an amino acid sequence of SEQ ID NO: 56;
e. the VH2 comprises an amino acid sequence of SEQ ID NO: 49, and wherein the V_{L2} comprises an amino acid sequence of SEQ ID NO: 57;
f. the VH2 comprises an amino acid sequence of SEQ ID NO: 50, and wherein the V_{L2} comprises an amino acid sequence of SEQ ID NO: 58;
g. the VH2 comprises an amino acid sequence of SEQ ID NO: 51, and wherein the V_{L2} comprises an amino acid sequence of SEQ ID NO: 59; or
h. the VH2 comprises an amino acid sequence of SEQ ID NO: 52, and wherein the V_{L2} comprises an amino acid sequence of SEQ ID NO: 60.

In some embodiments, the binding protein is characterized in that the binding protein comprises three polypeptide chains (I), (II) and (III) that form two ABDs, as defined below:

V_{1A}-C_{1A}-Hinge₁-(C_{H}2-C_{H}3)_{A} (I)

V_{1B}-C_{1B}-Hinge₂-(C_{H}2-C_{H}3)_{B}-L₁-V_{2A}-C_{2A}-Hinge₃ (II)

V_{B2}-C_{2B} (III)
wherein:
V_{1A} and V_{1B} form a binding pair V₁ (V_{H1}/V_{L1});
V_{2A} and V_{2B} form a binding pair V₂ (V_{H2}/V_{L2});
C_{1A} and C_{1B} form a pair C₁ (C_{H}1/C_{L}) and C_{2A} and C_{2B} form a pair C₂ (C_{H}1/C_{L}) wherein C_{H}1 is an immunoglobulin heavy chain constant domain 1 and C_{L} is an immunoglobulin light chain constant domain;
Hinge₁, Hinge₂ and Hinge₃ are identical or different and correspond to all or part of an immunoglobulin hinge region;
(C_{H}2-C_{H}3)_{A} and (C_{H}2-C_{H}3)_{B} are identical or different, and comprise an immunoglobulin heavy chain constant domain 2 (C_{H}2) and an immunoglobulin heavy chain constant domain 3 (C_{H}3);
L₁ is an amino acid linker.

In some embodiments, the binding protein is characterized in that it comprises a C_{1B} is an immunoglobulin heavy chain constant domain 1 (C_{H}1);
C_{2A} is an immunoglobulin heavy chain constant domain 1 (C_{H}1);
C_{L} corresponds to an immunoglobulin kappa light chain constant domain (C_{κ});
(C_{H}2-C_{H}3)_{A} corresponds to the amino acid sequence of SEQ ID NO: 69;
(C_{H}2-C_{H}3)_{B} corresponds to the amino acid sequence of SEQ ID NO: 70;
Hinge₁ corresponds to the amino acid sequence of SEQ ID NO:74;
Hinge₂ corresponds to the amino acid sequence of SEQ ID NO:75;
Hinge₃ corresponds to the amino acid sequence of SEQ ID NO: 77;
L₁ corresponds to the amino acid sequence of SEQ ID NO: 76.

In some embodiments, the binding protein is characterized in that the residue N297 of the Fc region or variant thereof according to EU numbering comprises a N-linked glycosylation.

In some embodiments, the binding protein is characterized in that the all or part of the Fc region or variant thereof binds to a human CD16A (FcγRIII) polypeptide. In some embodiments, the binding protein is characterized in that at least two polypeptide chains are linked by at least one disulfide bridge.

In some embodiments, the binding protein is characterized in that the polypeptide chains (I) and (II) are linked by at least one disulfide bridge between C_{1A} and Hinge₂ and/or wherein the polypeptide chains (II) and (III) are linked by at least one disulfide bridge between Hinge₃ and C_{2B}.

In some embodiments, the binding protein is characterized in that V_{1A} is V_{L1} and V_{1B} is V_{H1}.

In some embodiments, the binding protein is characterized in that V_{2A} is V_{H2} and V_{2B} is V_{L2}.

In some embodiments, the binding protein is characterized in that:
(a) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 13; a CDR-H2 comprising the amino acid sequence of SEQ ID NO: 14; a CDR-H3 comprising the amino acid sequence of SEQ ID NO: 15; V_{L2} comprises a CDR-L1 comprising the amino acid sequence of SEQ ID NO: 27; a CDR-L2 comprising the amino acid sequence of SEQ ID NO: 28; a CDR-L3 comprising the amino acid sequence of SEQ ID NO: 29;
(b) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 16; a CDR-H2 comprising the amino acid sequence of SEQ ID NO: 17; a CDR-H3 comprising the amino acid sequence of SEQ ID NO: 18; V_{L2} comprises a CDR-L1 comprising the amino acid sequence of SEQ ID NO: 30; a CDR-L2 comprising the amino acid sequence of SEQ ID NO: 31; a CDR-L3 comprising the amino acid sequence of SEQ ID NO: 32;
(c) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 19; a CDR-H2 comprising the amino acid sequence of SEQ ID NO: 20; a CDR-H3 comprising the amino acid sequence of SEQ ID NO: 21; V_{L2} comprises a CDR-L1 comprising the amino acid sequence of SEQ ID NO: 33; a CDR-L2 comprising the amino acid sequence of SEQ ID NO: 34; a CDR-L3 comprising the amino acid sequence of SEQ ID NO: 35;
(d) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 22; a CDR-H2 comprising the amino acid sequence of SEQ ID NO: 23; a CDR-H3 comprising the amino acid sequence of SEQ ID NO: 24; V_{L2} comprises a CDR-L1 comprising the amino acid sequence of SEQ ID NO: 36; a CDR-L2 comprising the amino acid sequence of SEQ ID NO: 37; a CDR-L3 comprising the amino acid sequence of SEQ ID NO: 38;
(e) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 16; a CDR-H2 comprising the amino acid sequence of SEQ ID NO: 25; a CDR-H3 comprising the amino acid sequence of SEQ ID NO: 26; V_{L2} comprises a CDR-L1 comprising the amino acid sequence of SEQ ID NO: 39; a CDR-L2 comprising the amino acid sequence of SEQ ID NO: 31; a CDR-L3 comprising the amino acid sequence of SEQ ID NO: 40;
(f) V_{H1} comprises a CDR-H1 comprising the amino acid sequence of SEQ ID NO: 4;
a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 13; a CDR-H2 comprising the amino acid sequence of SEQ ID NO: 14; a CDR-H3 comprising the amino acid sequence of SEQ ID NO: 15; V_{L2} comprises a CDR-L1 comprising the amino acid sequence of SEQ ID NO: 27; a CDR-L2 comprising the amino acid sequence of SEQ ID NO: 28; a CDR-L3 comprising the amino acid sequence of SEQ ID NO: 29;
(g) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 16; a CDR-H2 comprising the amino acid sequence of SEQ ID NO: 17; a CDR-H3 comprising the amino acid sequence of SEQ ID NO: 18; V_{L2} comprises a CDR-L1 comprising the amino acid sequence of SEQ ID NO: 30; a CDR-L2 comprising the amino acid sequence of SEQ ID NO: 31; a CDR-L3 comprising the amino acid sequence of SEQ ID NO: 32;
(h) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 19; a CDR-H2 comprising the amino acid sequence of SEQ ID NO: 20; a CDR-H3 comprising the amino acid sequence of SEQ ID NO: 21; V_{L2} comprises a CDR-L1 comprising the amino acid sequence of SEQ ID NO: 33; a CDR-L2 comprising the amino acid sequence of SEQ ID NO: 34; a CDR-L3 comprising the amino acid sequence of SEQ ID NO: 35;
(i) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 22; a CDR-H2 comprising the amino acid sequence of SEQ ID NO: 23; a CDR-H3 comprising the amino acid sequence of SEQ ID NO: 24; V_{L2} comprises a CDR-L1 comprising the amino acid sequence of SEQ ID NO: 36; a CDR-L2 comprising the amino acid sequence of SEQ ID NO: 37; a CDR-L3 comprising the amino acid sequence of SEQ ID NO: 38; or
(j) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 16; a CDR-H2 comprising the amino acid sequence of SEQ ID NO: 25; a CDR-H3 comprising the amino acid sequence of SEQ ID NO: 26; V_{L2} comprises a CDR-L1 comprising the amino acid sequence of SEQ ID NO: 39; a CDR-L2 comprising the amino acid sequence of SEQ ID NO: 31; a CDR-L3 comprising the amino acid sequence of SEQ ID NO: 40.

In some embodiments, the binding protein is characterized in that:
(a) V_{H1} and V_{L1} corresponds to the amino acid sequences of SEQ ID NO: 41 and 43 respectively or corresponds to the amino acid sequences of SEQ ID NO: 42 and 44 respectively;
and/or
(b) V_{H2} and V_{L2} corresponds to
   the amino acid sequences of SEQ ID NO: 45 and 53 respectively;
   the amino acid sequences of SEQ ID NO: 46 and 54 respectively;
   the amino acid sequences of SEQ ID NO: 47 and 55 respectively;
   the amino acid sequences of SEQ ID NO: 48 and 56 respectively;
   the amino acid sequences of SEQ ID NO: 49 and 57 respectively;
   the amino acid sequences of SEQ ID NO: 50 and 58 respectively;
   the amino acid sequences of SEQ ID NO: 51 and 59 respectively; or
   the amino acid sequences of SEQ ID NO: 52 and 60 respectively.

In some embodiments, the binding protein is characterized in that:
(a) V_{H1} comprises the amino acid sequence of SEQ ID NO: 41; V_{L1} comprises the amino acid sequence of SEQ ID NO: 43; V_{H2} comprises the amino acid sequence of SEQ ID NO: 45; V_{L2} comprises the amino acid sequence of SEQ ID NO: 53;
(b) V_{H1} comprises the amino acid sequence of SEQ ID NO: 41; V_{L1} comprises the amino acid sequence of SEQ ID NO: 43; V_{H2} comprises the amino acid sequence of SEQ ID NO: 46; V_{L2} comprises the amino acid sequence of SEQ ID NO: 54;
(c) V_{H1} comprises the amino acid sequence of SEQ ID NO: 41; V_{L1} comprises the amino acid sequence of SEQ ID NO: 43; V_{H2} comprises the amino acid sequence of SEQ ID NO: 47; V_{L2} comprises the amino acid sequence of SEQ ID NO: 55;
(d) V_{H1} comprises the amino acid sequence of SEQ ID NO: 41; V_{L1} comprises the amino acid sequence of SEQ ID NO: 43; V_{H2} comprises the amino acid sequence of SEQ ID NO: 48; V_{L2} comprises the amino acid sequence of SEQ ID NO: 56;
(e) V_{H1} comprises the amino acid sequence of SEQ ID NO: 41; V_{L1} comprises the amino acid sequence of SEQ ID NO: 43; V_{H2} comprises the amino acid sequence of SEQ ID NO: 49; V_{L2} comprises the amino acid sequence of SEQ ID NO: 57;
(f) V_{H1} comprises the amino acid sequence of SEQ ID NO: 41; V_{L1} comprises the amino acid sequence of SEQ ID NO: 43; V_{H2} comprises the amino acid sequence of SEQ ID NO: 50; V_{L2} comprises the amino acid sequence of SEQ ID NO: 58;
(g) V_{H1} comprises the amino acid sequence of SEQ ID NO: 41; V_{L1} comprises the amino acid sequence of SEQ ID NO: 43; V_{H2} comprises the amino acid sequence of SEQ ID NO: 51; V_{L2} comprises the amino acid sequence of SEQ ID NO: 59;
(h) V_{H1} comprises the amino acid sequence of SEQ ID NO: 41; V_{L1} comprises the amino acid sequence of SEQ ID NO: 43; V_{H2} comprises the amino acid sequence of SEQ ID NO: 52; V_{L2} comprises the amino acid sequence of SEQ ID NO: 60;
(i) V_{H1} comprises the amino acid sequence of SEQ ID NO: 42; V_{L1} comprises the amino acid sequence of SEQ ID NO: 44; V_{H2} comprises the amino acid sequence of SEQ ID NO: 45; V_{L2} comprises the amino acid sequence of SEQ ID NO: 53;
(j) V_{H1} comprises the amino acid sequence of SEQ ID NO: 42; V_{L1} comprises the amino acid sequence of SEQ ID NO: 44; V_{H2} comprises the amino acid sequence of SEQ ID NO: 46; V_{L2} comprises the amino acid sequence of SEQ ID NO: 54;
(k) V_{H1} comprises the amino acid sequence of SEQ ID NO: 42; V_{L1} comprises the amino acid sequence of SEQ ID NO: 44; V_{H2} comprises the amino acid sequence of SEQ ID NO: 47; V_{L2} comprises the amino acid sequence of SEQ ID NO: 55;
(l) V_{H1} comprises the amino acid sequence of SEQ ID NO: 42; V_{L1} comprises the amino acid sequence of SEQ ID NO: 44; V_{H2} comprises the amino acid sequence of SEQ ID NO: 48; V_{L2} comprises the amino acid sequence of SEQ ID NO: 56;
(m) V_{H1} comprises the amino acid sequence of SEQ ID NO: 42; V_{L1} comprises the amino acid sequence of SEQ ID NO: 44; V_{H2} comprises the amino acid sequence of SEQ ID NO: 49; V_{L2} comprises the amino acid sequence of SEQ ID NO: 57;
(n) V_{H1} comprises the amino acid sequence of SEQ ID NO: 42; V_{L1} comprises the amino acid sequence of SEQ ID NO: 44; V_{H2} comprises the amino acid sequence of SEQ ID NO: 50; V_{L2} comprises the amino acid sequence of SEQ ID NO: 58.
(o) V_{H1} comprises the amino acid sequence of SEQ ID NO: 42; V_{L1} comprises the amino acid sequence of SEQ ID NO: 44; V_{H2} comprises the amino acid sequence of SEQ ID NO: 51; V_{L2} comprises the amino acid sequence of SEQ ID NO: 59;
(p) V_{H1} comprises the amino acid sequence of SEQ ID NO: 42; V_{L1} comprises the amino acid sequence of SEQ ID NO: 44; V_{H2} comprises the amino acid sequence of SEQ ID NO: 52; V_{L2} comprises the amino acid sequence of SEQ ID NO: 60.

In some embodiments, the binding protein is characterized in that:
polypeptide (I) consists of an amino acid sequence of SEQ ID NO: 64;
polypeptide (II) consists of an amino acid sequence of SEQ ID NO: 65; and
polypeptide (III) consists of an amino acid sequence of SEQ ID NO: 66.

### II. BCL2 Inhibitors

As used herein, “BCL-2” or the “BCL-2 protein” refers to the first member of the BCL-2 protein family to be identified in humans i.e., B-cell lymphoma 2. Human BCL-2 plays a key role in inhibiting apoptosis and deregulation of BCL-2 (typically associated with apoptosis-resistance) can propel cancer growth. Human BCL-2 is encoded by the BCL2 gene (UniProtKB-P10415) and has the amino acid sequences shown under NCBI Reference Sequences NP_000624.2 and NP_000648.2.

BCL-2 has been found to be upregulated in several different types of cancer. For example, the genetic hallmark of follicular lymphoma chromosomal translocation t(14; 18)(q32; q21), which brings the BCL2 gene on chromosome 18 under the influence of the IgH promoter on chromosome 14, resulting in a constitutive overexpression of the antiapoptotic BCL2 protein. In another example, BCL-2 gene amplification has been reported in different cancers including leukemias (such as CLL), lymphomas (such as B-cell lymphoma) and some solid tumors (e.g., small-cell lung carcinoma). In another example, BCL-2 was overexpressed in patients with acute myeloid leukemia (AML) (87.3% of AML cases at onset and in 100% of relapse cases). In AML patients BCL-2 overexpression resulted in the deregulation of apoptosis corresponding leukemic cell protection from apoptosis.

As used herein, a “BCL-2 inhibitor” refers to any agent, compound, or molecule capable of specifically inhibiting the activity of BCL-2, in particular an agent, compound, or molecule capable of inhibiting the anti-apoptotic activity of BCL-2.

Examples of BCL-2 inhibitors suitable for use in combination treatment as described herein include a venetoclax (Waggoner et al. (2022), J Adv Pract Oncol., vol. 13 (4): 400-415), a ABT-737 (Parrondo et al. (2013), PeerJ, vol. 1: e144), a obatoclax (Chiappori et al. (2014), J Thorac Oncol., vol. 9 (1): 121-125), a pelcitoclax (APG-1252; Qin et al. (2022), Mol. Carcinog., vol. 61 (11): 1031-1042), a LP-118 (Ravikrishnan et al. (2021), Blood, vol. 138 (1): 679), a navitoclax (also known as “ABT-263”; Rudin et al. (2012), Clin Cancer Res., vol. 18 (11): 3163-3169), a BM-1197 (Bai et al. (2014), Plos One, vol. 9 (6): e99404), a BCL2-32 (Adam et al. (2014), Blood, vol. 124 (121): 5304), a AZD4320 (Balachander et al. (2020), Clin Cancer Res., vol. 26 (24): 6535-6549), a S55746 (Casara et al. (2018), Oncotarget, vol. 9 (28): 20075-20088), or a B cell lymphoma homology 3 (BH3) mimetic compound (Merino et al. (2018), Cancer Cell, vol. 34 (6): 879-891), e.g., oblimersen (G3139 BCL-2 antisense oligonucleotide, including the sodium salt of oblimersen, i.e., oblimersen sodium). Further examples of BCL-2 inhibitors are described in Ashkenazi et al. (2017), Nat Rev Drug Discovery, vol. 16 (4): 273-284; Ploumaki et al. (2023), Clin Transl Oncol., vol. 25 (6): 1554-1578 which are incorporated herein by reference.

In one aspect, a combination described herein comprises a BCL-2 inhibitor. In some embodiments, the combination comprises a BCL-2 inhibitor wherein the BCL-2 inhibitor is venetoclax, ABT-737, obatoclax, pelcitoclax, LP-118, navitoclax, BM-1197, BCL2-32, AZD4320, S55746, or a BH3 mimetic compound e.g., oblimersen sodium. In some embodiments, the combination comprises a BCL-2 inhibitor wherein the BCL-2 inhibitor is venetoclax, oblimersen sodium, obatoclax mesylate, palcitoclax, or LP-118.

In one aspect, the disclosure provides a method for treating or preventing a leukemia or a myelodysplastic syndrome in a subject in need thereof, said method comprising administering to the subject an effective amount of a combination comprising: (i) a binding protein comprising a first antigen binding domain (ABD) with binding specificity to CD123 and a second (ABD) with binding specificity to NKp46; and one or both of: (ii) a BCL-2 inhibitor wherein the BCL-2 inhibitor is venetoclax, a ABT-737, obatoclax, a pelcitoclax, a LP-118, navitoclax, a BM-1197, BCL2-32, AZD4320, S55746, or a BH3 mimetic compound e.g., oblimersen sodium, and (iii) a DNA hypomethylating agent.

As used herein, the term “venetoclax” refers to the compound having the chemical structure shown below:

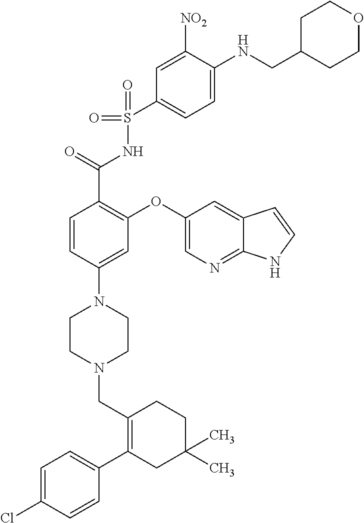 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

Venetoclax is a potent, selective, orally-bioavailable inhibitor of the BCL-2 protein. It has the empirical formula C₄₅H₅₀C1N₇O₇S and a molecular weight of 868.44. It has very low aqueous solubility. Venetoclax can be described chemically as 4-(4-{[2-(4-chlorophenyl)-4,4dimethylcyclohex-1-en-1-yl]methyl}piperazin-1-yl)-N-({3-nitro-4-[(tetrahydro-2H-pyran-4ylmethyl)amino]phenyl}sulfonyl)-2-(1H-pyrrolo[2,3-b]pyridin-5-yloxy)benzamide). Alternative names for venetoclax include ABT-199; chemical name 1257044-40-8; GDC-0199.

In one aspect, the disclosure provides a method for treating or preventing a leukemia or a myelodysplastic syndrome in a subject in need thereof, said method comprising administering to the subject an effective amount of a combination comprising: (i) a binding protein comprising a first antigen binding domain (ABD) with binding specificity to CD123 and a second (ABD) with binding specificity to NKp46; and one or both of:
(ii) a venetoclax or a pharmaceutically acceptable salt thereof and (iii) a DNA hypomethylating agent. In some embodiments, the combinations described herein comprise the following structure:

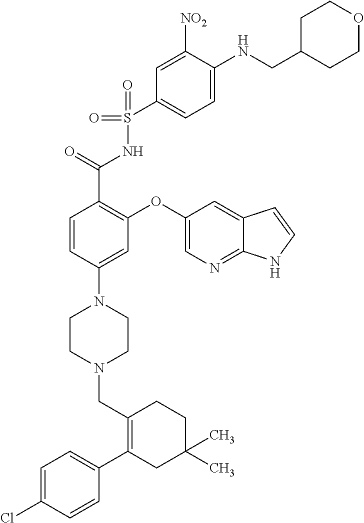 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

Venetoclax for use in the combination therapies described herein may be provided in any suitable form such that it effectively inhibits the BCL-2 protein. Such forms include but are not limited to any suitable polymorphic, amorphous or crystalline forms or any isomeric or tautomeric forms. In certain embodiments, the combination therapies described herein comprise venetoclax synthesized according to the process described in US2010/0305122, EP3333167, WO2017/156398, WO2018/029711, CN107089981 (A), WO2018/069941, WO2017/212431, WO2018/009444, CN107648185 (A), WO2018/167652, WO2018/157803, CZ201769, and WO2012/071336, which are herein incorporated herein by reference.

Pharmaceutically acceptable salts for ventoclax are generally known in the art and can include salts of acidic or basic groups. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Pharmaceutically acceptable salts may be formed with various amino acids. Suitable base salts include, but are not limited to, aluminum, calcium, lithium, magnesium, potassium, sodium, zinc, and diethanolamine salts. Venetoclax for use in the combination therapies described herein may also be provided in the form of a hydrate, anhydrate or solvate. In some embodiments, the methods as described in the current disclosure comprise venetoclax or a pharmaceutically acceptable salt thereof.

### III. Hypomethylating Agents (HMAs)

Epigenetic modifications, such as DNA methylation are a therapeutic target in hematopoietic malignancies. DNA methylation occurs through the covalent addition of a methyl group to the 5′ carbon of the cytosine ring catalyzed by DNA methyltransferases (DNMT), resulting in 5-methylcytosine. Common sites within the genome that can be methylated or demethylated are cytosine-guanine dinucleotides referred to as CpG dinucleotides. CpG islands (CGIs) are areas with a high concentration of CpG.

In cancer, aberrant and hyper DNA methylation at CGIs within promoter regions result in the silencing of critical tumor suppressor genes involved in cancer-related pathways, such as invasion, DNA repair, and cell cycle regulation. See Taby and Issa (2010), CA Cancer J Clin., vol. 60 (6): 376-392 as well as Jelinek et al. (2012), Epigenetics, vol. 7 (12); 1368-1378. Approximately five to ten percent of promoter CGIs undergo hypermethylation in most cancers, leading to the silencing of crucial tumor-suppressor genes like VHL, RB1, CDKN2A, MGMT, GATA4, and MLH1. Id. For example, in colon cancer, CGI methylator phenotype correlates with BRAF mutations. Suzuki et al. (2014), Biochem Biophys Res Comun, vol. 455 (1-2): 35-42. Additionally, hypermethylation and/or mutations in protein and enzymes that regulate DNA methylation, like DNMT3A, IDH, and TET are prevalent in cancers, e.g., in hematological malignancies. Yang et al. (2015), Nature Reviews Cancer, vol. 15:152-165. For example, in AML patients exhibit TET2 or IDH mutations with distinct hypermethylation signatures. Figueroa et al. (2010), J Cancer Cell, vol. 17 (1): 13-27.

The term “hypomethylating agent” also known as “HMAs” refers to a class of compounds that interfere with DNA or RNA methylation. In the context of cancer therapy, HMAs are compounds that can inhibit methylation, resulting in the expression of the previously hypermethylated silenced genes which can in turn restore normal cell functions and sensitize tumor cells to chemotherapy and immunotherapy. Two HMA driven mechanisms to antitumor activity are: (i) cytotoxicity due to incorporation into RNA and/or DNA, leading to induction of DNA damage response and (ii) DNA hypomethylation through inhibition of DNA methyltransferase, enabling restoration of normal cell growth and differentiation.

Examples of hypomethylating agents suitable for use in a combination treatment described herein include an azacitidine (also known as 5-aza-2′-deoxycytidine, 5-Azacytidine, Azacytidine, Ladakamycin, 4-Amino-1-β-D-ribofuranosyl-s-triazin-2(1H)-one, and U-18496) (Venegas et al. (2022), Cancer Manag Res, vol. 14:3527-3538), a cytidine, a decitabine, a guadecitabine (Stomper et al. (2021), Leukemia, vol 35(7): 1873-1889), a 5-fluoro-2′-deoxycytidine (Zhao et al. (2012), SpringerPlus, vol. 1:65), a zebularine (Lai et al. (2021), Mol. Ther, vol. 29(5): 1758-1771), a RG108 (Ou et al. Oncol. Rep., vol. 39(3): 993-1002), a nanaomycin A (Nakashima et al. (2017), J Biosci Bioeng, vol. 123(6): 765-770), a guadecitabine (Daher-Reyes et al. (2019), Expert Opinion Inventig Drug, vol. 28(10): 835-849), a RX-3117 (Sarkisjan et al. (2020), Int J Mol Sci, vol 21(8): 2717), a CC-486 (Wei et al. (2020), N Eng J Med., vol. 383(26): 2526-2537, or a ASTX727 (Garcia-Manero et al. Am J Hematol, vol 95(11): 1399-1420). Further examples of hypomethylating agents are described in Lui et al. (2021), J Hemaltol Oncol. vol 14(1): 49 which is incorporated by reference.

In one aspect, a combination described herein comprises a hypomethylating agent. In some embodiments, the combination comprises an azacitidine, a cytidine, a decitabine, a guadecitabine, a 5-fluoro-2′-deoxycytidine, a zebularine, a RG108, a nanaomycin A, a guadecitabine, a RX-3117, a CC-486, or a ASTX727. In some embodiments, the combination comprises an azacitidine, a cytidine, a decitabine, a guadecitabine.

In one aspect, the disclosure provides a method for treating or preventing a leukemia or a myelodysplastic syndrome in a subject in need thereof, said method comprising administering to the subject an effective amount of a combination comprising: (i) a binding protein comprising a first antigen binding domain (ABD) with binding specificity to CD123 and a second (ABD) with binding specificity to NKp46; and one or both of: (ii) a BCL-2 inhibitor, and (iii) a DNA hypomethylating agent wherein the DNA hypomethylating agent is an azacitidine, a cytidine, a decitabine, or a guadecitabine.

5-Azacitidine (also known as AZA or azavitidine), is a pyrimidine nucleoside analogue of cytidine. It is a white crystalline powder with the empirical formula C8H12N4O5 and a molecular weight of 244.2 g/mol. The CAS Registry Number for 5-azacitidine is 320-67-2. 5-azacitidine chemical name is 4-amino-1-beta-D-ribofuranosyl-s-triazin-2 (1H)-one and has the following chemical structure:

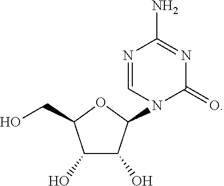 [SMILES: Nc1ncn([C@@H]2O[C@H](CO)[C@@H](O)[C@H]2O)c(=O)n1]

In one aspect, the disclosure provides a method for treating or preventing a leukemia or a myelodysplastic syndrome in a subject in need thereof, said method comprising administering to the subject an effective amount of a combination comprising: (i) a binding protein comprising a first antigen binding domain (ABD) with binding specificity to CD123 and a second (ABD) with binding specificity to NKp46; and one or both of: (ii) a BCL-2 inhibitor and (iii) a 5-azacytidine or a pharmaceutically acceptable salt thereof. In some embodiments, the combinations described herein comprise the following structure:

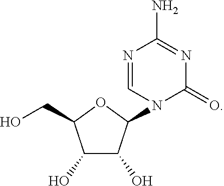 [SMILES: Nc1ncn([C@@H]2O[C@H](CO)[C@@H](O)[C@H]2O)c(=O)n1]

5-azacytidine for use in the combination therapies described herein may be provided in any suitable form such that it effectively inhibits DNA methyltransferase activity. Such forms include but are not limited to any suitable polymorphic, amorphous or crystalline forms or any isomeric or tautomeric forms. In certain embodiments, the combination therapies described herein comprise 5-azacytidine synthesized according to the process described in WO2004082619A2, WO2012135405A1, WO2009139888A1, WO2006034154A2 which are herein incorporated herein by reference.

Pharmaceutically acceptable salts for 5-azacytidine are generally known in the art and can include salts of acidic or basic groups. Pharmaceutically acceptable acid addition salts include, but are not limited to, hydrochloric, L-lactic, acetic, phosphoric, (+)-L-tartaric, citric, propionic, butyric, hexanoic, L-aspartic, L-glutamic, succinic, EDTA, maleic, niethanesulfonic acid, HBr, HF, HI, nitric, nitrous, sulfuric, sulfurous, phosphorous, perchloric, chloric, chlorous acid, carboxylic acid, a sulfonic acid, ascorbic, carbonic, and fumaric acid, a hydrochloride, mesylate, EDTA, sulfite, L-Aspartate, maleate, phosphate, L-Glutamate, (+)-L-Tartrate, citrate, L-Lactate, succinate, acetate, hexanoate, butyrate, or propionate salt. In some embodiments, the methods as described in the current disclosure comprise 5-azacytidine or a pharmaceutically acceptable salt thereof.

### IV. Combination Therapies

As used herein, the term “combination therapy” refers to a method of prevention or treatment in which a subject in need thereof, for example a human subject, is administered a combination of two or more therapeutic agents. These “combinations” can comprise (i) a binding protein of the disclosure (e.g., a CD123 NKCE) and a BCL-2 inhibitor; or (ii) a binding protein of the disclosure and a DNA hypomethylating; or (iii) a binding protein of the disclosure, a BCL-2 inhibitor, and a DNA hypomethylating agent. The combinations may be co-formulated for administration or may be provided separately, for example as separate compositions, for administration to a subject or patient in need thereof. In some embodiments, the combinations have additive activity. In other embodiments, the combinations have synergistic activity.

In some embodiments, wherein the binding protein of the disclosure, the BCL-2 inhibitor, and the DNA hypomethylating agent are administered simultaneously or concurrently. In some embodiments, wherein the binding protein of the disclosure, the BCL-2 inhibitor, and the DNA hypomethylating agent are administered sequentially. For example, the BLC-2 inhibitor and/or DNA hypomethylating agent may be administered before or after the binding protein of the disclosure.

For example, when administered “before” the binding protein of the disclosure, the BCL-2 inhibitor and/or the DNA hypomethylating agent may be administered about 1 month, about 3 weeks, about 2 weeks, about 1 week, about 7 days, about 6 days, about 5 days, about 4 days, about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes, or about 10 minutes prior to the administration of the binding protein of the disclosure. When administered “after” the binding protein of the disclosure, the additional therapeutic agent may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours, about 72 hours, about 4 days, about 5 days, about 6 days, about 7 days, about 1 week, about 2 weeks, about 3 weeks, or about 1 month after the administration of the binding protein of the disclosure. “Concurrent” administration comprising the binding protein of the disclosure means that the BCL-2 inhibitor and/or the DNA hypomethylating agent is administered to the subject in a separate dosage form within less than 5 minutes (before, after, or at the same time) of administration of the binding protein of the disclosure, or administered to the subject as a single combined dosage formulation comprising the BCL-2 inhibitor and/or the DNA hypomethylating agent and the binding protein of the disclosure or as separate formulations, one comprising the binding protein of the disclosure and the other comprising the BCL-2 inhibitor and/or the DNA hypomethylating agent.

In some embodiments, the binding protein is administered to the subject intravenously, subcutaneously, intraperitoneally, or intramuscularly. In some embodiments, the binding protein is administered to the subject intravenously. In some embodiments, the binding protein is administered to the subject intravenously weekly for induction followed by every 4 weeks for maintenance. In some embodiments, one or both of the BCL-2 inhibitor and/or the DNA hypomethylating agent are administered to the subject intravenously, subcutaneously, intraperitoneally, or intramuscularly. In some embodiments, the binding protein, the BCL-2 inhibitor, and the DNA hypomethylating agent have the same route of administration. In some embodiments, the binding protein, the BCL-2 inhibitor, and the DNA hypomethylating agent have different routes of administration. In some embodiments, the BCL-2 inhibitor is administered orally. In some embodiments, the DNA hypomethylating agent is administered intravenously or subcutaneously.

As demonstrated in the Examples, combinations of the disclosure exhibit synergistic tumor cell lysis, that is, the level of inhibition induced by the combination is greater than the additive effect of the monotherapies alone. The synergistic efficacy on tumor cell lysis of the combination therapies of the disclosure are envisaged to be used as a method for treating or preventing hematological diseases (e.g., a leukemia or a myelodysplastic syndrome) in a subject in need thereof.

In certain embodiments, a combination therapy of the disclosure is a method of treating or preventing a hematological neoplastic disease or disorder in a subject. Hematological neoplastic diseases or disorders include blood cancers, including tumors of the hematopoietic and lymphoid tissues, such as lymphomas, myelomas, and leukemias. Leukemias include, but are not limited to acute myeloid leukemia, acute lymphoblastic leukemia, chronic lymphocytic leukemia, chronic myelogenous leukemia, and hairy cell leukemia.

In certain embodiments, a combination therapy of the disclosure is a method of treating or preventing a hematological disease or disorder in a subject in need thereof comprising administering to the subject a therapeutically effective amount of the CD123 NKCE of the present disclosure in combination with one or both of: a BCL-2 inhibitor, and a DNA hypomethylating agent.

In some embodiments, the combination agents (i.e., a binding protein disclosed herein with one or both a BCL-2 inhibitor and a DNA hypomethylating agent) of the present disclosure can be particularly useful in the treatment of a disease or disorder within the category of leukemias or myelodysplasias. In some embodiments, the leukemia is AML. In some embodiments, the leukemia is B-ALL. In some embodiments, the myelodysplasia is high-risk myelodysplasia (HR-MDS).

A therapeutically effective dose of the NK cell engager disclosed herein (e.g., CD123 NKCE), in combination with one or both a BCL-2 inhibitor, and a DNA hypomethylating agent may be the dose or amount sufficient to induce a “therapeutic response” in a subject, which as an improvement in at least one measure of a hematological disease or disorder (e.g., AML, ALL, B-ALL, or HR-MDS).

In certain embodiments, a combination therapy of the disclosure is method of treating or preventing AML in a subject. As used herein, “Acute myelogenous leukemia (AML)” is a clonal disorder clinically presenting as increased proliferation of heterogeneous and undifferentiated myeloid blasts. Without wishing to be bound by the theory, the leukemic hierarchy is maintained by a small population of LSCs (Leukemic Stem Cells) (AML-LSCs), which have the distinct ability for self-renewal, and are able to differentiate into leukemic progenitors. These progenitors generate the large numbers of leukemic blasts readily detectable in patients at diagnosis and relapse, leading ultimately to mortality. AML-LSC have been commonly reported as quiescent cells, in contrast to rapidly dividing clonogenic progenitors.

In certain embodiments, the AML subject is a newly diagnosed de novo AML patient.

In certain embodiments, the AML subject is ineligible for intensive chemotherapy, e.g., standard intensive induction therapy such cytosine arabinoside and anthracycline based induction therapy. In certain embodiments, the AML patient is ineligible for intensive chemotherapy if they are elderly (>75 years). In other embodiments, the AML patients are ineligible for intensive chemotherapy if have comorbidities or poor patient fitness that precludes aggressive chemotherapy induction. Exemplary comorbidities which preclude intensive chemotherapy include a history of congestive heart failure for which treatment was warranted, an ejection fraction of 50% or less, chronic stable angina, a diffusing capacity of the lung for carbon monoxide of 65% of less, or a forced expiratory volume in 1 second of 65% or less, or an Easter Cooperative Oncology Group (ECOG) performance-status score of 2 or 3.

In other embodiments, the AML subject has previously received intensive induction therapy, e.g., cytosine arabinoside and anthracycline based induction therapy, followed by consolidation chemotherapy with cytarabine or other agents. In certain embodiments, the AML subject has failed to respond to the previously received intensive induction therapy.

In certain embodiments, the AML subject has relapsed AML. Within the context of AML, the term “relapse” may in particular be defined as the reoccurrence of AML after complete remission. In that sense “complete remission” or “CR” may be defined as follows: normal values for neutrophil (>1.0*109/L), haemoglobin level of 10 g/dl and platelet count (>100*109/L) and independence from red cell transfusion; blast cells less than 5%, no clusters or collections of blasts, and absence of Auer rods on bone marrow examination; and normal maturation of blood cells (morphology; myelogramme) and absence of extramedullary leukemia.

In certain embodiments, the AML subject has refractory AML. As used herein, the term “refractory” means the cancer did not respond to treatment. In the context of AML, most patients achieve a remission (an absence of signs and symptoms) after initial treatment. However, some patients have residual leukemic cells in their marrow even after intensive treatment. Patients who have not achieved complete remission after two cycles of induction chemotherapy are usually diagnosed as having “refractory AML.”

In certain embodiment, the AML patients have with relapsed or refractory disease (r/r) following initial therapies; e.g., AML patients with relapsed or refractory disease (r/r) following initial therapy, e.g., intensive induction chemotherapy and/or allogeneic stem cell transplantation for AML. AML patients with relapsed or refractory disease (r/r) following initial therapy exhibit a poor prognosis. Despite the development of several new agents for r/r AML, it is usually incurable. A short duration of remission (e.g., <6 months), adverse genetic factors, prior allogeneic transplantation, older age, and general health status are factors associated with worse survival outcomes in the setting of r/r AML, in part because these factors limit eligibility for further intensive therapy.

In certain embodiments, a combination therapy of the disclosure is method of treating or preventing a myelodysplastic syndrome in a subject. As used herein, “myelodysplastic syndromes” (“MDS”), formerly known as preleukemia, are a collection of hematological conditions that involve ineffective production (or dysplasia) of the myeloid class of blood cells. They represent a spectrum of clonal hematopoietic stem cell disorders characterized by progressive bone marrow failure and increased risk of progression to acute myeloid leukemia (“AML”, also known as “acute myelogenous leukemia”). The International Prognostic Scoring System (“IPSS”) is widely used to identify patients with high-risk features based on the severity of their cytopenias, bone marrow myeloblast percentage, and cytogenetic abnormalities.

In certain embodiments, the B-ALL or HR-MDS patients are elderly patients (>75 years) or patients that have comorbidities that preclude aggressive chemotherapy induction. In other embodiments the B-ALL or HR-MDS patients who have exhibited remission following allogeneic stem cell transplantation. In other embodiments, the B-ALL or HR-MDS patient is resistant to a CD19-directed therapy, such as tisagenlecleucel or blinatumomab, a T-cell engaging antibody targeting CD19.

In another embodiment, the combination agents (i.e., a binding protein disclosed herein with one or both a BCL-2 inhibitor and a DNA hypomethylating agent) are useful for the treatment of diseases or disorders associated with aberrant immune cells, e.g., myeloid cells, B cells. In some embodiments, the aberrant immune cells, e.g., myeloid cells or B cells, express CD123.

In one aspect, the disclosure relates to (i) a binding protein comprising a first antigen-binding domain (ABD) comprising a variable region which binds specifically to a human CD123 polypeptide and a second antigen-binding domain (ABD) comprising a variable region which binds specifically to a human NKp46 polypeptide, and one or both of: (ii) a BCL-2 inhibitor, and (iii) a DNA hypomethylating agent; for use in a method for the treatment or prevention of blood cancer.

In one aspect, the disclosure relates to (i) a binding protein comprising a first antigen-binding domain (ABD) comprising a variable region which binds specifically to a human CD123 polypeptide and a second antigen-binding domain (ABD) comprising a variable region which binds specifically to a human NKp46 polypeptide, and one or both of: (ii) a BCL-2 inhibitor, and (iii) a DNA hypomethylating agent; for use in a method for the treatment or prevention of a myelodysplastic syndrome (MDS) or of a lymphoproliferative disorder.

In one aspect, the disclosure relates to (i) a binding protein comprising a first antigen-binding domain (ABD) comprising a variable region which binds specifically to a human CD123 polypeptide and a second antigen-binding domain (ABD) comprising a variable region which binds specifically to a human NKp46 polypeptide, and one or both of: (ii) a BCL-2 inhibitor, and (iii) a DNA hypomethylating agent; for use in a method for the treatment or prevention of Acute Myeloid Leukemia (AML).

In one aspect, the disclosure relates to (i) a binding protein comprising a first antigen-binding domain (ABD) comprising a variable region which binds specifically to a human CD123 polypeptide and a second antigen-binding domain (ABD) comprising a variable region which binds specifically to a human NKp46 polypeptide, and one or both of: (ii) a BCL-2 inhibitor, and (iii) a DNA hypomethylating agent; for use in a method for the treatment or prevention of CD64-positive and CD64-negative Acute Myeloid Leukemia (AML).

In another aspect, the disclosure relates to a binding protein comprising a first and a second antigen binding domains (ABDs) and all or part of an immunoglobulin Fc region or variant thereof, wherein each of said ABDs comprises an immunoglobulin heavy chain variable domain (VH) and an immunoglobulin light chain variable domain (VL), wherein each VH and VL comprises three complementary determining regions (CDR-1 to CDR-3); and wherein:
(i) the first ABD binds specifically to human CD123 and comprises:
a V_{H1} comprising a CDR-H1, H2 and H3 corresponding to the amino acid sequences SEQ ID NO:1 to 3 respectively or corresponding to the amino acid sequences of SEQ ID NO: 4 to 6 respectively, and
a V_{L1} comprising a CDR-L1, L2 and L3 corresponding to the amino acid sequences of SEQ ID NO: 7 to 9 respectively or corresponding to the amino acid sequences of SEQ ID NO: 10 to 12 respectively;
(ii) the second ABD binds specifically to human NKp46 and comprises:
a V_{H2} comprising a CDR-H1, 2 and 3 corresponding to:
the amino acid sequences of SEQ ID NO:13 to 15 respectively;
the amino acid sequences of SEQ ID NO:16 to 18 respectively;
the amino acid sequences of SEQ ID NO:19 to 21 respectively;
the amino acid sequences of SEQ ID NO:22 to 24 respectively; or the amino acid sequences of SEQ ID NO:16, 25 and 26 respectively;
and
a V_{L2} comprising a CDR-L1, 2 and 3 corresponding to:
the amino acid sequences of SEQ ID NO:27 to 29 respectively;
the amino acid sequences of SEQ ID NO:30 to 32 respectively;
the amino acid sequences of SEQ ID NO:33 to 35 respectively;
the amino acid sequences of SEQ ID NO:36 to 38 respectively; or
the amino acid sequences of SEQ ID NO:31, 39 and 40 respectively;
   wherein all or part of the immunoglobulin Fc region or variant thereof binds to a human Fc-γ receptor,
   and one or both of: a BCL-2 inhibitor and a DNA hypomethylating agent; for use in a method for the treatment or prevention of blood cancer.

In another aspect, the disclosure relates to a binding protein comprising a first and a second antigen binding domains (ABDs) and all or part of an immunoglobulin Fc region or variant thereof, wherein each of said ABDs comprises an immunoglobulin heavy chain variable domain (V_{H}) and an immunoglobulin light chain variable domain (V_{L}), wherein each V_{H} and V_{L} comprises three complementary determining regions (CDR-1 to CDR-3); and wherein:
(i) the first ABD binds specifically to human CD123 and comprises:
a V_{H1} comprising a CDR-H1, H2 and H3 corresponding to the amino acid sequences of SEQ ID NO:1 to 3 respectively or corresponding to the amino acid sequences of SEQ ID NO: 4 to 6 respectively, and
a V_{L1} comprising a CDR-L1, L2 and L3 corresponding to the amino acid sequences of SEQ ID NO: 7 to 9 respectively or corresponding to the amino acid sequences of SEQ ID NO: 10 to 12 respectively;
(ii) the second ABD binds specifically to human NKp46 and comprises:
a V_{H2} comprising a CDR-H1, 2 and 3 corresponding to:
the amino acid sequences of SEQ ID NO:13 to 15 respectively;
the amino acid sequences of SEQ ID NO:16 to 18 respectively;
the amino acid sequences of SEQ ID NO:19 to 21 respectively;
the amino acid sequences of SEQ ID NO:22 to 24 respectively; or
the amino acid sequences of SEQ ID NO:16, 25 and 26 respectively;
and
a V_{L2} comprising a CDR-L1, 2 and 3 corresponding to:
the amino acid sequences of SEQ ID NO:27 to 29 respectively;
the amino acid sequences of SEQ ID NO:30 to 32 respectively;
the amino acid sequences of SEQ ID NO:33 to 35 respectively;
the amino acid sequences of SEQ ID NO:36 to 38 respectively; or
the amino acid sequences of SEQ ID NO:31, 39 and 40 respectively;
   wherein all or part of the immunoglobulin Fc region or variant thereof binds to a human Fc-γ receptor,
   and one or both of: a BCL-2 inhibitor and a DNA hypomethylating agent; for use in a method for the treatment or prevention of a myelodysplastic syndrome (MDS) or of a lymphoproliferative disorder.

In another aspect, the disclosure relates to a binding protein comprising a first and a second antigen binding domains (ABDs) and all or part of an immunoglobulin Fc region or variant thereof, wherein each of said ABDs comprises an immunoglobulin heavy chain variable domain (V_{H}) and an immunoglobulin light chain variable domain (V_{L}), wherein each V_{H} and V_{L} comprises three complementary determining regions (CDR-1 to CDR-3); and wherein:
(i) the first ABD binds specifically to human CD123 and comprises:
a V_{H1} comprising a CDR-H1, H2 and H3 corresponding to the amino acid sequences of SEQ ID NO:1 to 3 respectively or corresponding to the amino acid sequences of SEQ ID NO: 4 to 6 respectively, and
a V_{L1} comprising a CDR-L1, L2 and L3 corresponding to the amino acid sequences SEQ ID NO: 7 to 9 respectively or corresponding to the amino acid sequences SEQ ID NO: 10 to 12 respectively;
(ii) the second ABD binds specifically to human NKp46 and comprises:
a V_{H2} comprising a CDR-H1, 2 and 3 corresponding to:
the amino acid sequences of SEQ ID NO:13 to 15 respectively;
the amino acid sequences of SEQ ID NO:16 to 18 respectively;
the amino acid sequences of SEQ ID NO:19 to 21 respectively;
the amino acid sequences of SEQ ID NO:22 to 24 respectively; or
the amino acid sequences of SEQ ID NO:16, 25 and 26 respectively;
and
a V_{L2} comprising a CDR-L1, 2 and 3 corresponding to:
the amino acid sequences of SEQ ID NO:27 to 29 respectively;
the amino acid sequences of SEQ ID NO:30 to 32 respectively;
the amino acid sequences of SEQ ID NO:33 to 35 respectively;
the amino acid sequences of SEQ ID NO:36 to 38 respectively; or
the amino acid sequences of SEQ ID NO:31, 39 and 40 respectively;
   wherein all or part of the immunoglobulin Fc region or variant thereof binds to a human Fc-γ receptor,
   and one or both of: a BCL-2 inhibitor and a DNA hypomethylating agent; for use in a method for the treatment or prevention of Acute Myeloid Leukemia (AML).

In another aspect, the disclosure relates to a binding protein comprising a first and a second antigen binding domains (ABDs) and all or part of an immunoglobulin Fc region or variant thereof, wherein each of said ABDs comprises an immunoglobulin heavy chain variable domain (V_{H}) and an immunoglobulin light chain variable domain (V_{L}), wherein each V_{H} and V_{L} comprises three complementary determining regions (CDR-1 to CDR-3); and wherein:
(i) the first ABD binds specifically to human CD123 and comprises:
a V_{H1} comprising a CDR-H1, H2 and H3 corresponding to the amino acid sequences of SEQ ID NO:1 to 3 respectively or corresponding to the amino acid sequences of SEQ ID NO: 4 to 6 respectively, and
a V_{L1} comprising a CDR-L1, L2 and L3 corresponding to the amino acid sequences of SEQ ID NO: 7 to 9 respectively or corresponding to the amino acid sequences of SEQ ID NO: 10 to 12 respectively;
(ii) the second ABD binds specifically to human NKp46 and comprises:
a V_{H2} comprising a CDR-H1, 2 and 3 corresponding to:
the amino acid sequences of SEQ ID NO:13 to 15 respectively;
the amino acid sequences of SEQ ID NO:16 to 18 respectively;
the amino acid sequences of SEQ ID NO:19 to 21 respectively;
the amino acid sequences of SEQ ID NO:22 to 24 respectively; or
the amino acid sequences of SEQ ID NO:16, 25 and 26 respectively;
and
a V_{L2} comprising a CDR-L1, 2 and 3 corresponding to:
the amino acid sequences of SEQ ID NO:27 to 29 respectively;
the amino acid sequences of SEQ ID NO:30 to 32 respectively;
the amino acid sequences of SEQ ID NO:33 to 35 respectively;
the amino acid sequences of SEQ ID NO:36 to 38 respectively; or
the amino acid sequences of SEQ ID NO:31, 39 and 40 respectively;
   wherein all or part of the immunoglobulin Fc region or variant thereof binds to a human Fc-γ receptor,
   and one or both of: a BCL-2 inhibitor and a DNA hypomethylating agent; for use in a method for the treatment or prevention of CD64-positive and CD64-negative Acute Myeloid Leukemia (AML).

The disclosure further relates to a use of said combination agents (i.e., a binding protein of the disclosure in combination with one or both a BCL-2 inhibitor and a DNA hypomethylating agent) for the preparation of a medicament for the treatment or prevention of cancer characterized by tumor cells that express CD123 at their surface. The disclosure further relates to a use of said combination agents for the preparation of a medicament for the treatment or prevention of cancer characterized by tumor cells that express CD123 and CD64 at their surface.

The disclosure further relates to a use of the above-mentioned combination agents for the preparation of a medicament for the treatment or prevention of blood cancer. The disclosure further relates to a use of said combination agents for the preparation of a medicament for the treatment or prevention of blood cancer characterized by tumor cells that express CD123 at their surface. The disclosure further relates to a use of said combination agents for the preparation of a medicament for the treatment or prevention of blood cancer characterized by tumor cells that express CD123 and CD64 at their surface.

The disclosure further relates to a use of the said combination agents for the preparation of a medicament for the treatment or prevention of a myelodysplastic syndrome (MDS) or of a lymphoproliferative disorder. The disclosure further relates to a use of the said combination agents for the preparation of a medicament for the treatment or prevention of Acute Myeloid Leukemia (AML). The disclosure further relates to a use of the said combination agents for the preparation of a medicament for the treatment or prevention of CD64-positive and CD64-negative Acute Myeloid Leukemia (AML).

In one aspect, provided is a method for treating a cancer characterized by tumor cells that express CD123 and CD64 at their surface, the method comprising administering to and individual having such cancer (i) a binding protein comprising a first antigen binding domain (ABD) with binding specificity to CD123 and a second (ABD) with binding specificity to NKp46; and one or both of: (ii) a BCL-2 inhibitor, and (iii) a DNA hypomethylating agent.

In one aspect, provided is method for treating a CD123-expressing tumor (e.g., a hematological malignancy, AML) in an individual who is susceptible to having tumor cells that express CD64 at their surface, the method comprising administering to the individual (i) a binding protein comprising a first antigen binding domain (ABD) with binding specificity to CD123 and a second (ABD) with binding specificity to NKp46; and one or both of: (ii) a BCL-2 inhibitor, and (iii) a DNA hypomethylating agent.

In one aspect, provided is method for treating a hematological malignancy (e.g., AML) in an individual, the method comprising administering to the individual (i) a binding protein comprising a first antigen binding domain (ABD) with binding specificity to CD123 and a second (ABD) with binding specificity to NKp46; and one or both of: (ii) a BCL-2 inhibitor, and (iii) a DNA hypomethylating agent.

In another aspect, provided is a method of treating a hematological malignancy (e.g., AML) in an individual, the method comprising: (a) assessing or determining whether malignant cells (e.g. AML cells) from the individual express CD64 at their surface; and (b) if the individual is determined to have malignant cells (e.g., AML cells) expressing CD64 at their surface (e.g., at a predetermined level), administering to the individual (i) a binding protein comprising a first antigen binding domain (ABD) with binding specificity to CD123 and a second (ABD) with binding specificity to NKp46; and one or both of: (ii) a BCL-2 inhibitor, and (iii) a DNA hypomethylating agent.

In another aspect, provided is a method for depleting malignant cells in an individual, and/or directing NK cell-mediated cytotoxicity toward CD64-expressing malignant cells (e.g., an individual having AML), the method comprising administering, to an individual having malignant cells (e.g., AML cells) expressing CD64 at their surface, (i) a binding protein comprising a first antigen binding domain (ABD) with binding specificity to CD123 and a second (ABD) with binding specificity to NKp46; and one or both of: (ii) a BCL-2 inhibitor, and (iii) a DNA hypomethylating agent.

In another aspect, provided is a method of causing NK cells to eliminate malignant cells that express both CD123 and CD64, the method comprising bringing the malignant cells (e.g., AML cells) into contact, in the presence of NK cells, with a combination comprising: (i) a binding protein comprising a first antigen binding domain (ABD) with binding specificity to CD123 and a second (ABD) with binding specificity to NKp46; and one or both of: (ii) a BCL-2 inhibitor, and (iii) a DNA hypomethylating agent.

In one aspect, the disclosure provides a method of treating or preventing a leukemia or a myelodysplastic syndrome in a subject in need thereof, said method comprising administering to the subject an effective amount of a combination comprising:
(i) a binding protein comprising a first ABD with binding specificity to CD123 and a second ABD with binding specificity to NKp46,
wherein the first ABD comprises a VH1 and a VL1, wherein:
   the VH1 comprises a complementary determining region (CDR)-H1, H2 and H3 corresponding to the amino acid sequences of SEQ ID NO: 1, 2, and 3; and
   the VL1 comprises a CDR-L1, L2 and L3 corresponding to the amino acid sequences of SEQ ID NO: 7, 8, and 9;
wherein the second antigen binding domain comprises a heavy chain variable domain (VH2) and a light chain variable domain (VL2), wherein:
   the VH2 comprises a CDR-H1, H2, and H3 corresponding to the amino acid sequences of SEQ ID NO: 13, 14, and 15;
   the VL2 comprises a CDR-L1, L2, and L3 corresponding to the amino acid sequences of SEQ ID NO: 27, 28, and 29; and
wherein all or part of the immunoglobulin Fc region or variant thereof binds to a human Fc-γ receptor;
(ii) a venetoclax or a pharmaceutically acceptable salt thereof; and
(iii) a 5-azacytidine or a pharmaceutically acceptable salt thereof,
   wherein the leukemia or a myelodysplastic syndrome is AML, B-ALL, or HR-MDS.

In another aspect, the disclosure provides a method of treating or preventing a leukemia or a myelodysplastic disorder in a subject in need thereof, said method comprising administering to the subject an effective amount of a combination comprising:
(i) a binding protein comprising a first antigen binding domain with binding specificity to CD123, a second antigen binding domain with binding specificity to NKp46, and all or part of an immunoglobulin Fc region or variant thereof that binds to a human Fc-γ receptor, wherein the binding protein comprises:
a polypeptide (I) that consists of an amino acid sequence of SEQ ID NO: 64;
a polypeptide (II) that consist of an amino acid sequence of SEQ ID NO: 65; and
a polypeptide (III) that consists of an amino acid sequence of SEQ ID NO: 66;
(ii) a venetoclax or a pharmaceutically acceptable salt thereof; and
(iii) a 5-azacytidine or a pharmaceutically acceptable salt thereof,
   wherein the leukemia or a myelodysplastic syndrome is AML, B-ALL, or HR-MDS.

### EXAMPLES

### Materials & Methods

### A.1. MOLM-13 Cell Line.

The MOLM-13 cell line (DSMZ ACC 554) is an acute myeloid leukemia cell line. These cells are mostly round cells growing in suspension. For maintenance, cells were resuspended in fresh RPMI1640 medium supplemented with 10% Fetal Bovin Serum (FBS; Biowest, ref #S140H-100) and 2 mM L-Glutamine (Gibco, ref #25030-024) referred as complete culture medium at 0.3×106 cells/mL for 3 or 4 days.

### A.2. MOLM-13 RFP Cell Line.

MOLM-13 RFP cells were generated by infecting MOLM-13 (DSMZ, ACC: 554) with the Incucyte® Nuclight Red Lentivirus (Sartorius #4476) to express a nuclear-restricted mKate2 (red fluorescent protein, RFP). MOLM-13 RFP cells were harvested in the same culture medium used for wild-type cells with the addition of 1 μg/mL of puromycin (Gibco, ref #A11138-03) to maintain the selection pressure.

### A.3. PBMC Preparation and NK Cell Collection

Fresh human peripheral blood mononuclear cells (PBMC) were isolated from healthy donors' whole blood samples supplied by EFS Ile de France.

Human PBMC were isolated from whole blood of healthy donors (HD) through a density gradient centrifugation.

Whole blood was recovered from the blood bag and diluted with 40 mL of sterile Phosphate Buffer Solution (PBS). 15 mL of Ficoll-Paque Plus Cytiva (Sigma Aldrich, ref #17-1440-02) were dispensed into the center of four sepMate-50 tubes (Stemcell ref #15450). Then, 80 mL of the diluted blood were gently added on the edge of each sepMate-50 tube containing Ficoll solution (20 mL per tube). The tubes were centrifuged at 1200 g for 20 minutes at room temperature (RT) without brake. The four buffy coat layers were recovered and collected in two 50 mL tubes (ThermoFisher Scientific, ref #339653). The leucocytes solution was completed with sterile PBS up to 50 mL final volume. The two tubes were centrifuged twice (in between each centrifugation, the supernatant was discarded, and 50 mL of PBS added) at 400 g for 10 minutes at RT with brake. After the last centrifugation, the pellets were mixed, and the volume completed to 10 mL by complete culture medium. The total viable PBMC number was defined by Vicell XR counting (Beckman Coulter cell counter instrument).

Human NK cells were purified from PBMCs using MACSxpress® Whole Blood NK Cell Isolation Kit (Miltenyi, ref #130-127-695) according to supplier recommendations. NK cells were then cultured in complete medium at 5×10⁶ cells/mL at +37° C. at 5% CO₂ (so called “resting” NK cells) overnight before being used in the activation assay.

### A.4. Product Preparation

Stock solutions of isotype control (an anti-Nkp46 antibody) and CD123 NKCE were stored in PBS at 4° C. On the day of the assay, the products were vortexed to eliminate potential aggregates before cascade dilution. A first dilution of 1/10 for each antibody was performed in complete culture medium. Then, for the CD123 NKCE a dilution of 1/400 was performed to obtain a final concentration of 100 ng/ml and from this latter a further dilution of 1/20 was done to get 5 ng/mL as final concentration. The isotype control was used only at the final concentration of 100 ng/mL.

Stock solution of AZA was stored in PBS at −20° C. On the day of the assay, the product was thawed and vortexed to eliminate potential aggregates before cascade dilution. A first dilution of 1/100 was performed in complete culture medium. Then, a dilution range from 20 μM (twice concentrated) to 20 pM (1/10 serial dilution) was performed in complete culture medium to assess the concentration-dependent effect of the single agent. Instead, to evaluate the combinatory effect of the two agents (AZA+VEN), a dilution range from 40 μM (4 times concentrated) to 40 pM was performed in complete culture medium.

Of these serial dilutions, 100 μL of AZA (single agent) was added to each well that contains 100 μL of cell suspension (MOLM-13 alone, NK cells alone or in coculture) to obtain a final concentration of 10, 1, 0.1, 0.01, 0.001, 0.0001, 0.00001 UM in a final volume of 200 L/well. To evaluate the combinatory effect of the two agents, 50 μL of AZA was added to each well that contains 100 μL of cell suspension and 50 μL of VEN (at four defined concentrations of 10, 1, 0.1, 0.01 μM) in a final volume of 200 μL/well.

Similarly, stock solution of VEN was stored in complete culture medium at 4° C. On the day of the assay, the product was vortexed to eliminate potential aggregates before cascade dilution. A dilution range from 20 μM (twice concentrated) to 20 pM (1/10 serial dilution) was performed in complete culture medium to assess the concentration-dependent effect of the single agent. To evaluate the combinatory effect of the two agents (VEN+AZA), a dilution range from 40 μM (4 times concentrated) to 40 pM was performed in complete culture medium.

Of these serial dilutions 100 μL of VEN (single agent) was added to each well that contains 100 μL of cell suspension to obtain a final concentration of 10, 1, 0.1, 0.01, 0.001, 0.0001, 0.00001 μM in a final volume of 200 μL/well. To evaluate the combinatory effect of the two agents, 50 μL of VEN were added to each well that will contain 100 μL of cell suspension and 50 μL of AZA (at four defined concentrations of 10, 1, 0.1, 0.01 μM) in a final volume of 200 μL/well.

For combinatory studies of the three agents in long-term cytotoxic assay, the concentrations used for CD123 NKCE were 100 and 5 ng/ml (defined as suboptimal concentrations); for AZA 600 nM (reaching the plasma concentration observed in patients); for VEN 2 nM (suboptimal concentration).

### A.5. Flow Cytometry-Based Assay

Following the proper timing of incubation (from 6 h to 72 h), 0.05×10⁶ cells/well MOLM-13 and NK cells in 96-well round bottom plates (Corning® Costar® Ultra-Low Attachment Multiple 96-Well Plate; Thermo Scientific, Denmark, ref #7007) were centrifuged at 300 g for 5 minutes at RT. Supernatant was aspirated and Tampon Running Buffer (Miltenyi, ref #130-091-2221) or the antibodies were added to the cell pellets (50 μL/well). In addition, 1 μL of each antibody was used to stain the compensation beads. The cells and the beads were incubated for 20 minutes at 4° C. Two consecutive washing steps were performed by adding 150 and then 200 μL of Tampon Running Buffer in the wells followed by centrifugation at 300 g for 5 minutes at 4° C.

Finally, 100 μL/well of PBS were added before reading the plate by MACSQuant Analyzer 16 Flow Cytometer. The propidium iodide (PI) marker of viability was added to every well reading at 1/10 dilution.

### A.6. 5-Azacytidine and Venetoclax Flow Cytometry Cytotoxic Assays

To define the optimal concentration to use in the following combinatory assays, AZA and VEN were tested in serial dilution alone or together with the other drug (at 4 different concentrations) and the viability of the AML cell line was assessed. CD123-positive MOLM-13 target cells were counted the day of the assay and the amount of cell needed for the experiment was evaluated. Target cells were seeded at 0.05×10⁶ cells/50 μL of complete culture medium per well.

In the meantime, compound seral dilutions or the four defined concentrations were prepared and added to a U-bottom 96-well plate (100 μL/well). As controls, target cells alone without drugs, were cultured at the same concentration in complete culture medium (200 μL/well). Each condition was performed in monoplicate.

Culture plates were incubated at 37° C. in the presence of 5% CO₂ for 48 h. The plates were centrifuged at 300 g for 5 minutes at RT and resuspended in 100 μL of PBS to the following reading by MACSQuant Analyzer 16 Flow. The marker of viability PI was added to every well reading at dilution 1/10.

### A.7. 5-Azacytidine and Venetoclax Cytotoxic Assays in the Presence of NK Cells

To assess the effect of AZA or VEN upon short conditioning in the presence of NK cells, a concentration showing low killing effect (1 nM) and a concentration showing high cytotoxic potential (1 μM) were chosen.

As previously, CD123-positive MOLM-13 target cells were counted and seeded at the concentration of 0.05×10⁶ cells/well and defined concentrations of AZA or VEN were added.

Finally, NK cell at effector:target (E:T) ratio of 1:1 (thus 0.05×10⁶ cells/well) were incubated with MOLM-13 cell line at 37° C. in the presence of 5% CO₂. After 6 h, 24 h, or 48 h, plates were centrifuged at 300 g for 5 minutes at RT. The Tampon Running Buffer or the selected antibodies were added to the cell pellets (50 μL/well) to assess cell viability and to perform the flow cytometry-based staining.

A.8. Combinatory Anti-Tumor Effects of 5-Azacytidine and/or Venetoclax with CD123 NKCE

The cytotoxic activity of CD123 NKCE combined with AZA and/or VEN was assessed using human NK cells purified from healthy donor blood as effector cells. The MOLM-13 RFP AML cell line exhibiting a CD123 antigen density representative of the patient population was selected as target cells for this assay.

Cytotoxicity was evaluated using the Incucyte live-cells analysis system which allows to quantify the number of live fluorescent target cells over time.

First, 50 μL of CD123 NKCE and its isotype control were distributed in the appropriate wells of an F-bottom (flat-bottom) 96-well plate coated with poly-D-lysine (Greiner BioOne ref #655946). CD123 NKCE was tested at 5 ng/mL, while its isotype was evaluated at the highest concentration of 100 ng/ml.

Subsequently, 50 μL of AZA and/or VEN were distributed in the appropriate wells in order to have final concentrations of 600 nM for AZA and 2 nM for VEN.

Then, MOLM-13 RFP cells (in 50 μL/well) and resting effector NK cells (in 50 μL/well) were successively added in each well to obtain an E:T ratio of 1:1 (0.03×10⁶ target cells and 0.03×10⁶ NK cells).

For control wells (MOLM-13 RFP cells alone or MOLM-13 RFP cells+NK cells without molecules) complete culture medium was added to reach the final volume of 250 μL/well. Each condition was performed in duplicate.

The growth/proliferation of MOLM-13 RFP tumor cells was monitored by cell imaging up to 64 h using the Incucyte® S3 with standard scans every 4 h. The Incucyte S3 software was used to analyze images. Tumor cells growth/proliferation was calculated by counting the number of red cells at each time point, which was then normalized to the number of cells at time zero and expressed as a percentage.

### Results

B1. Effect of 5-AZACYTIDINE and/or VENETOCLAX Treatment on AML Cell Viability

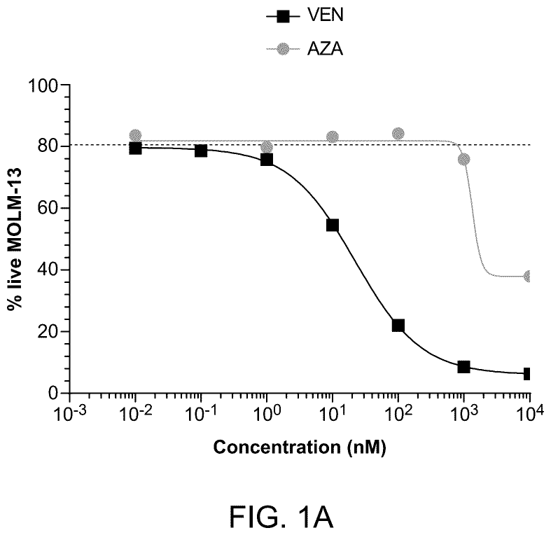

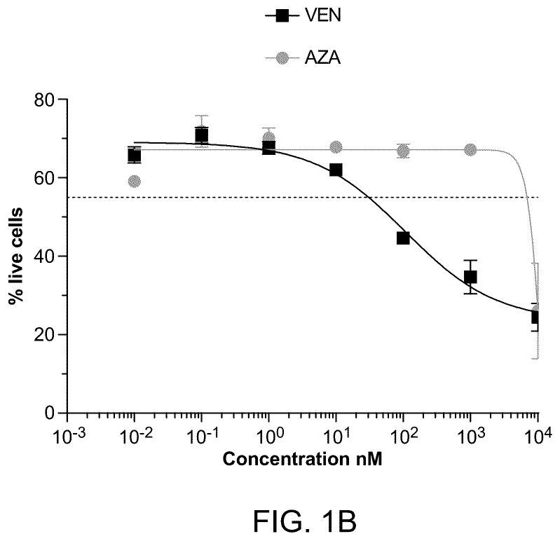

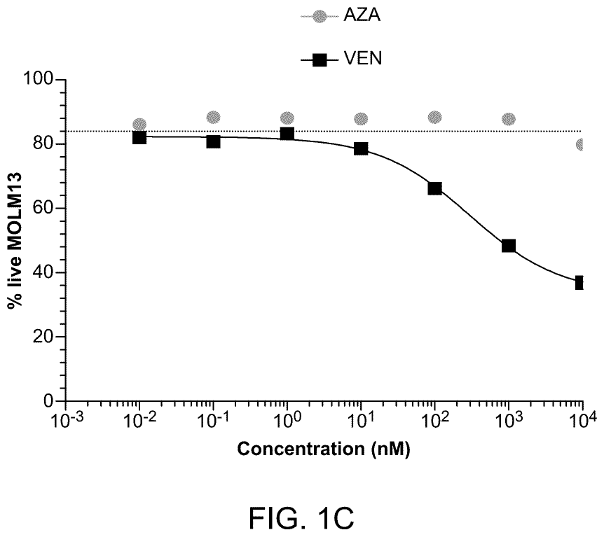

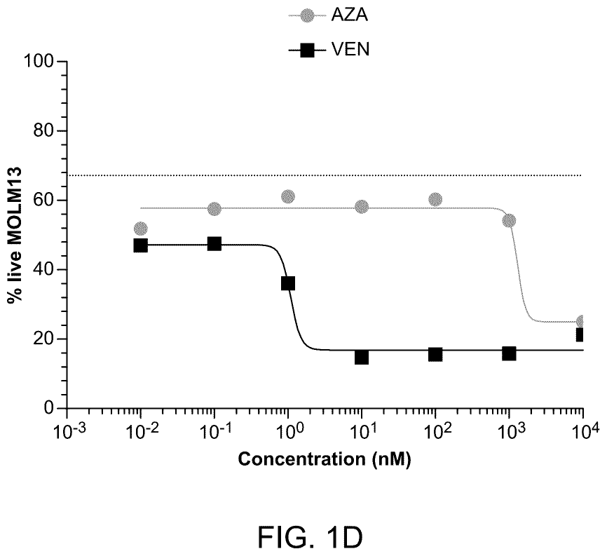

The in vitro anti-tumor efficacy of AZA and VEN as single agents was evaluated by measuring their cytotoxic effects against CD123-positive MOLM-13 AML cell line after a 48 h treatment in a concentration-dependent manner. Four independent experiments (replicates) were performed and the concentration-dependent response results as shown in FIG. 1A-1D. The addition of AZA in the culture medium strongly decreased AML cell viability at high concentration (10 μM). No effect was shown at lower concentrations of AZA compared to non-treated control MOLM-13 cells. A sigmoidal concentration-dependent response was observed for VEN from 0.01 nM to 10 μM with an impact on MOLM-13 cell viability from 10 nM to 10 μM concentrations.

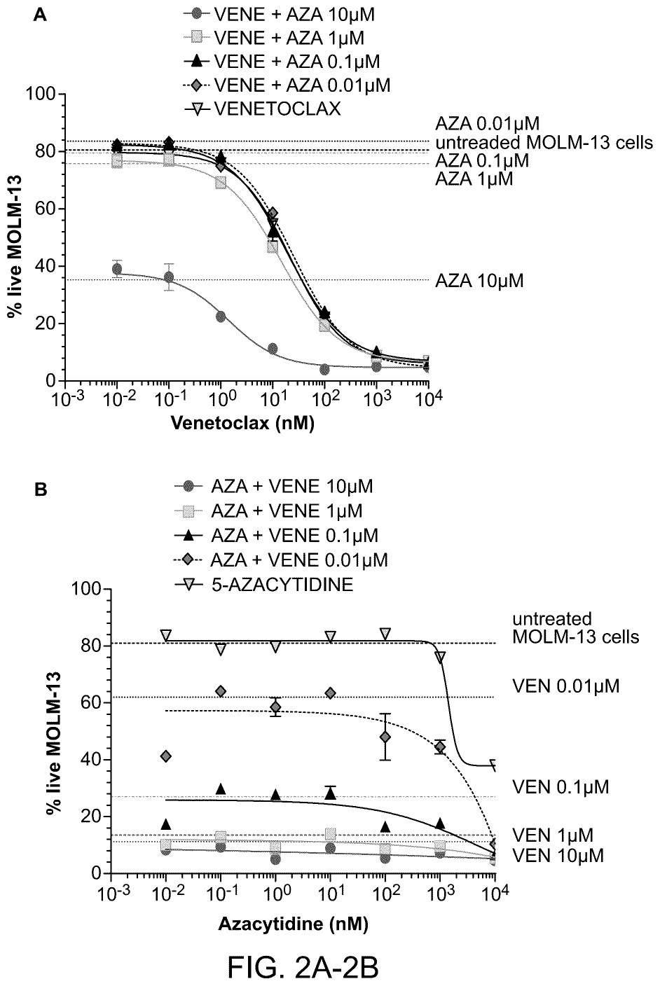

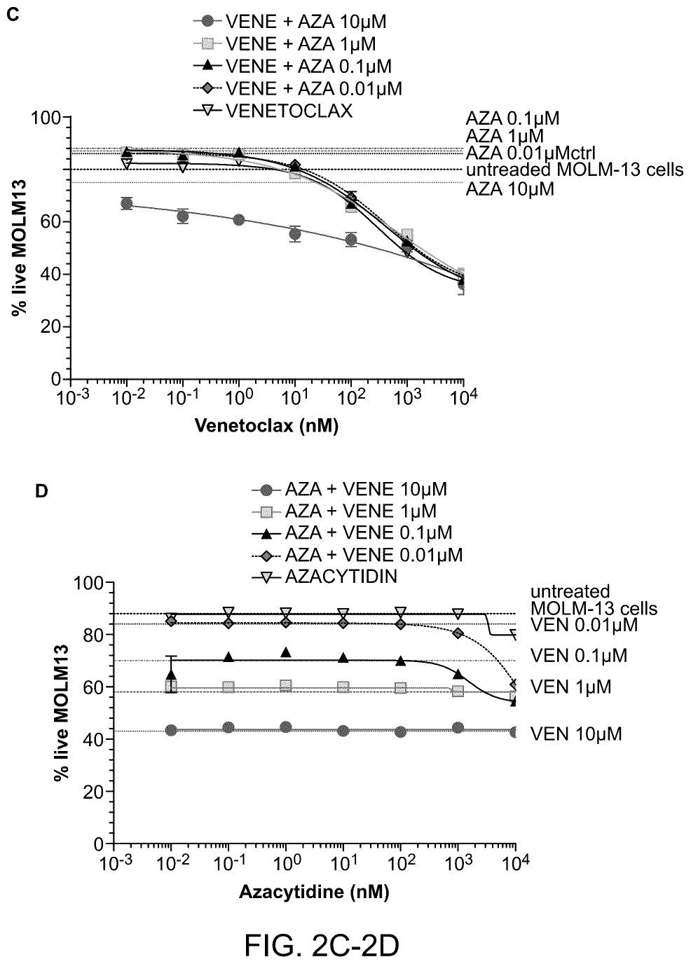

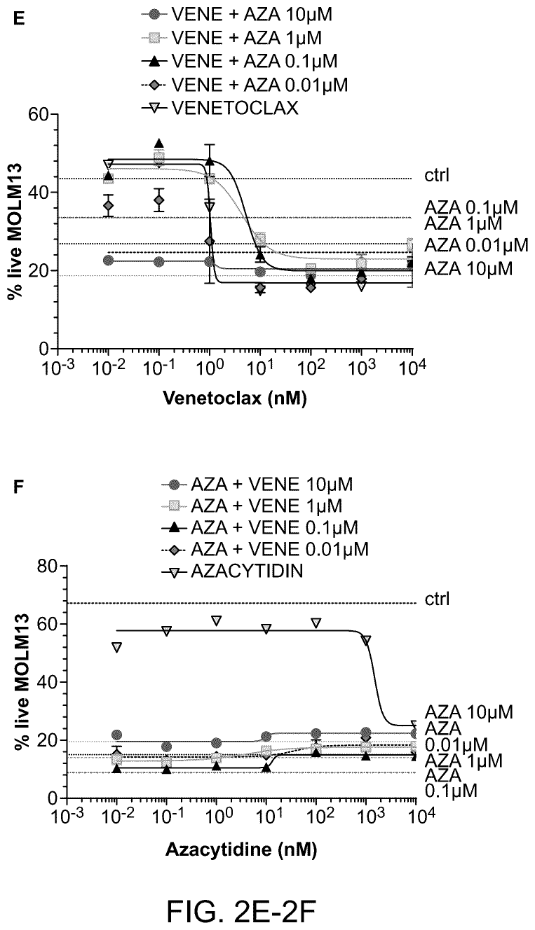

Moreover, AZA+VEN combination resulted in an increased induction of tumor cell killing after 48 h as compared to single agent treatment (VEN or AZA), confirming added benefit shown in literature. Jin S et al. (2020), Clin Cancer Res., vol. 26(13):3371-83. Three replicate experiments for each VEN or AZA were performed and the concentration-dependent response results are shown in FIG. 2A-2F.

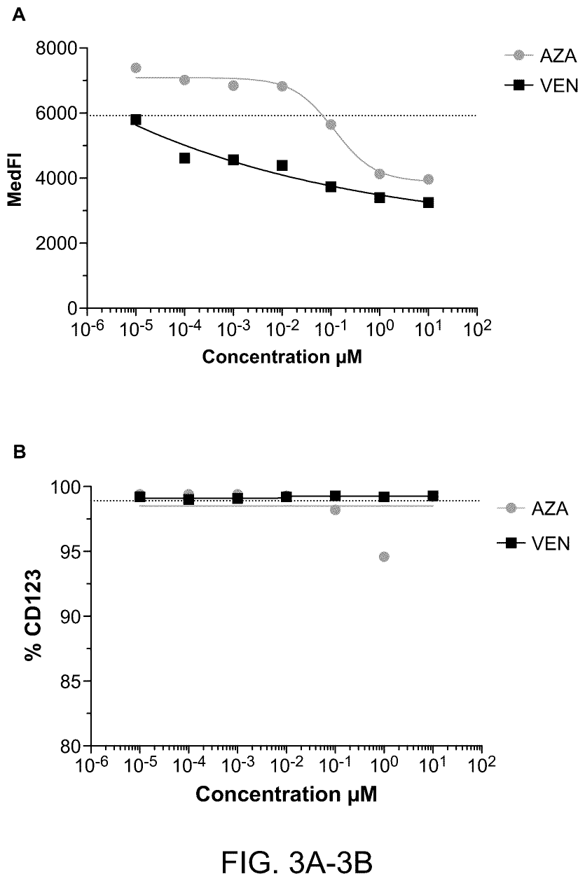

CD123 expression was evaluated in MOLM-13 cells at increasing concentrations of AZA or VEN ranging from 0.01 nM to 10 μM after treatment for 48 h. The addition of AZA showed minimal impact on CD123 expression level with only a slight decrease of the median of fluorescence intensity (MedFI) at 1 and 10 μM. The addition of VEN led to a mild reduction of CD123 (MedFI) on MOLM-13 cells after 48 h of treatment in a concentration-dependent manner as shown in FIG. 3.

Based on these results, the sub-optimal concentrations of AZA or VEN selected for combination studies with CD123 NKCE, were 600 nM AZA which corresponds to the plasma peak concentration observed in patients treated at the dose of 75 mg/m² (Derissen E J et al. (2013), Oncologist, vol. 18(5):619-24) and 1 or 2 nM VEN. The plasma concentration of VEN (2 μM at the dose of 400 mg) was not used for combination studies as it already induces a very strong killing activity as a single agent.

### B2. Effect of 5-AZACYTIDINE or VENETOCLAX on AML Cell Viability in a Time-Dependent Manner at Low and High Concentrations in the Absence or Presence of NK Cells

To evaluate the effects of AZA or VEN at different time points (6 h, 24 h, 48 h) on AML cells in presence of NK cells, a concentration showing low killing effect (1 nM) and a concentration showing high cytotoxic potential (1 μM) were chosen based on previous results. See FIG. 1A-1D and FIG. 2A-2F.

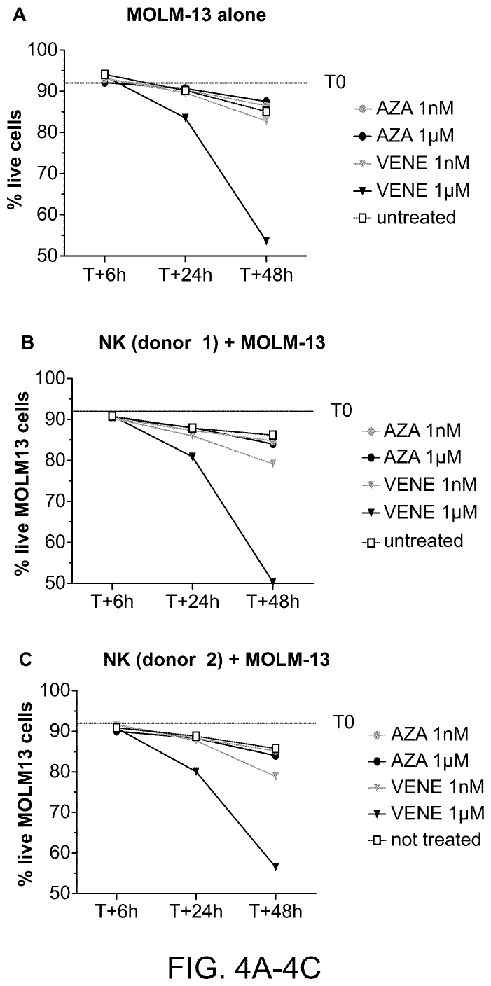

In presence of AZA at both tested concentrations and for any time point, the viability of MOLM-13 cells was similar to the one observed in the non-treated control samples. The treatment with VEN showed an enhanced killing potential at the concentration of 1 μM: this effect was time-dependent with a strong impact observed after treatment for 48 h and moderate to no impact after treatment for 24 h and 6 h, respectively (FIG. 4A-4C).

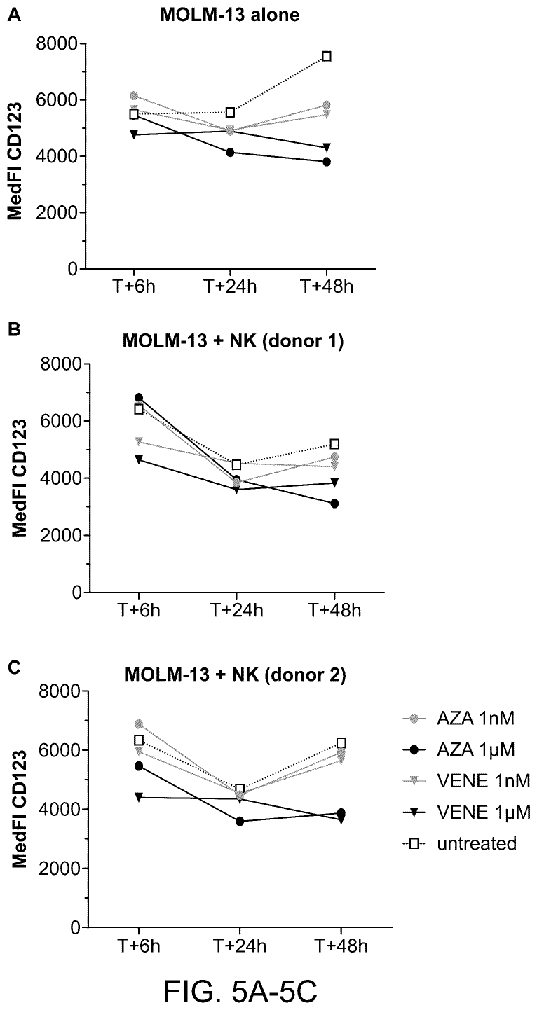

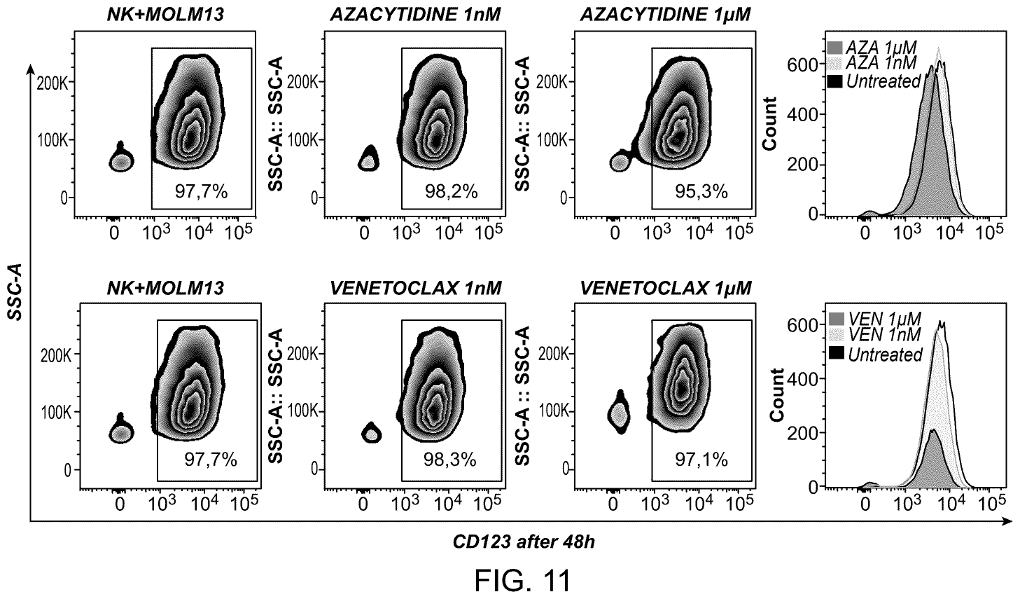

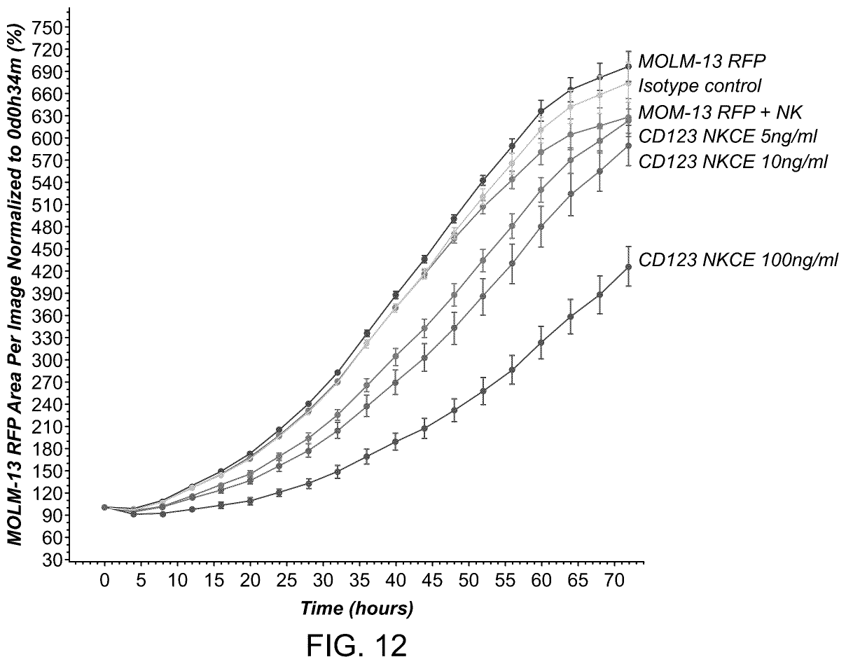

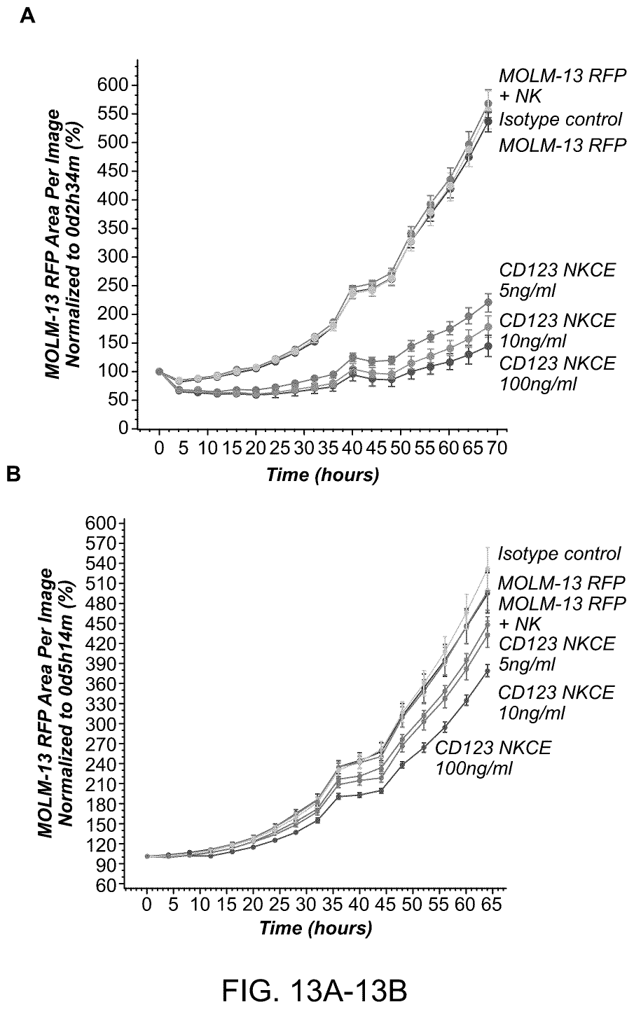

The CD123 expression in MOLM-13 cells treated with 1 nM and 1 μM of AZA or VEN was evaluated after 6 h, 24 h or 48 h of treatment. FIG. 5A-5C show that VEN and AZA did not impact CD123 expression (median fluorescence intensity) on MOLM-13 AML cells after treatment for up to 48 h in vitro at the tested concentrations, either on MOML-13 cells alone or in the presence of NK cells from two different donors. The corresponding flow cytometric plots are shown in FIG. 11. FIG. 11 shows expression of CD123 on MOML-13 cells with AZA (top panels) or VEN (bottom panels).

### B3. Impact of 5-AZACYTIDINE OR VENETOCLAX on NK Cells

The purpose of this Example was to evaluate the cytotoxic effect of AZA and/or VEN combined with CD123 NKCE. The potential impact of AZA and/or VEN treatments on CD123-positive MOLM-13 AML cells as well as on healthy donor (HD)-derived NK cells was investigated.

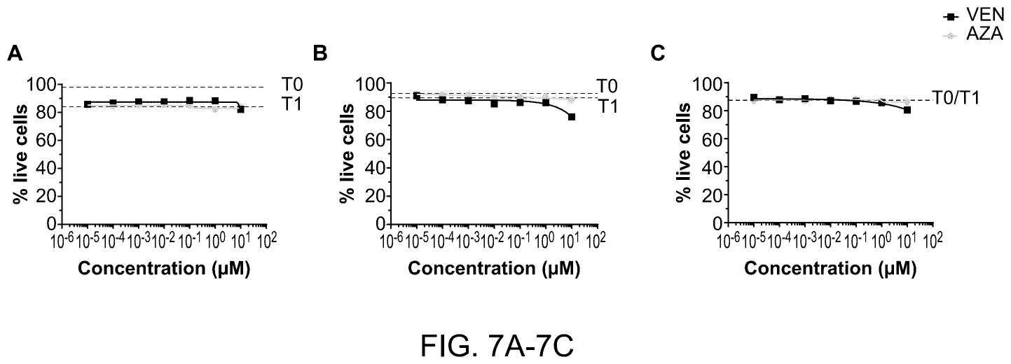

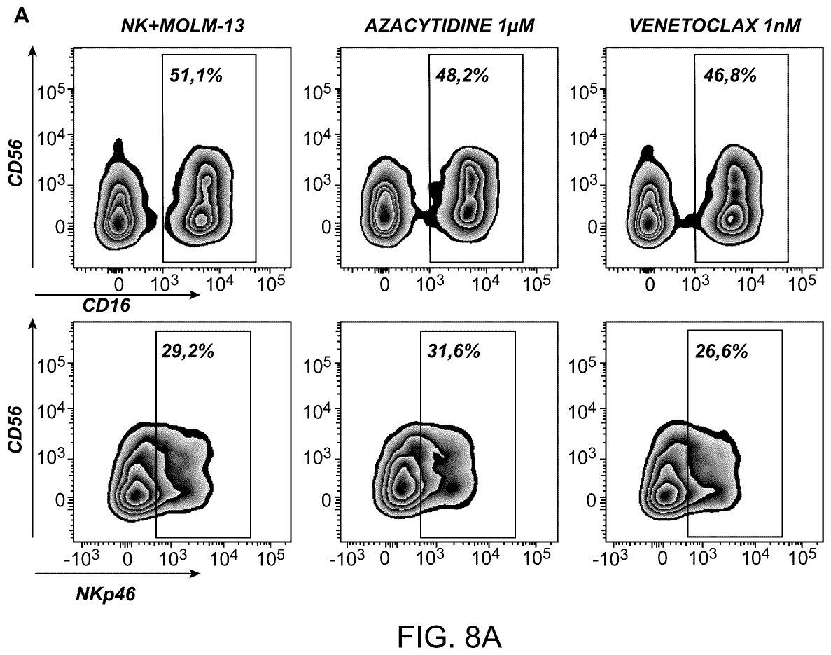

NK cell viability was assessed after treatment for 48 h with a single agent (AZA or VEN ranging from 0.01 nM to 10 μM concentrations) on 3 independent NK donors as shown in FIG. 7A-7C. Data indicate that AZA and/or VEN did not negatively impact NK cell viability. Moreover, CD16a and NKp46 expression levels were estimated in the presence of 1 μM of AZA or 1 nM of VEN (approaching the concentrations used in following combination studies). No impact on the expression of NKp46 and CD16a expression levels was observed (data not shown).

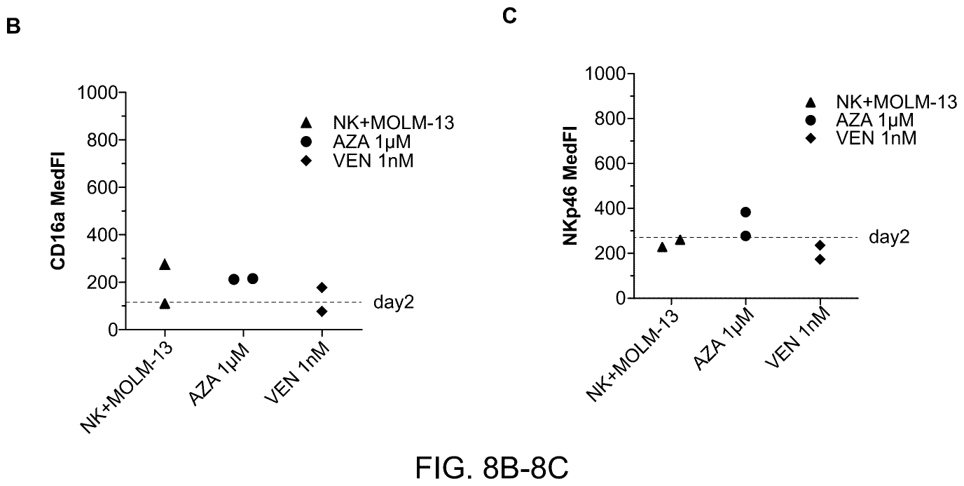

The addition of AZA or VEN at defined concentrations had minimal impact on NK cell phenotype, supporting the rationale of combining these drugs with CD123 NKCE (FIG. 8A-8C).

B4. In Vitro CD123 NKCE Cytotoxic Activity Against MOLM-13 AML Cells: Benefit of the Combination with 5-AZACYTIDINE and VENETOCLAX

Co-engagement of NK cells through binding to NKp46 and CD16a and CD123-positive tumor cells by CD123 NKCE leads to tumor cell killing.

The cytotoxic activity of CD123 NKCE alone or in combination with AZA and/or VEN was assessed using human NK cells purified from healthy donor blood as effector cells. The MOLM-13 RFP AML cells exhibiting a CD123 antigen density of around 20,000 antigens per cell were selected for this assay. The cytotoxic effect was evaluated quantifying the number of live fluorescent target cells over 64 h of incubation of tumor cells with single agents, double or triple combinations at an E:T ratio of 1:1. This experimental set-up was performed using NK cells derived from several independent healthy donors and the results of these experiments are discussed below in Example B5-B7.

### B5. Cytotoxic Effect of CD123 NKCE as a Single Agent

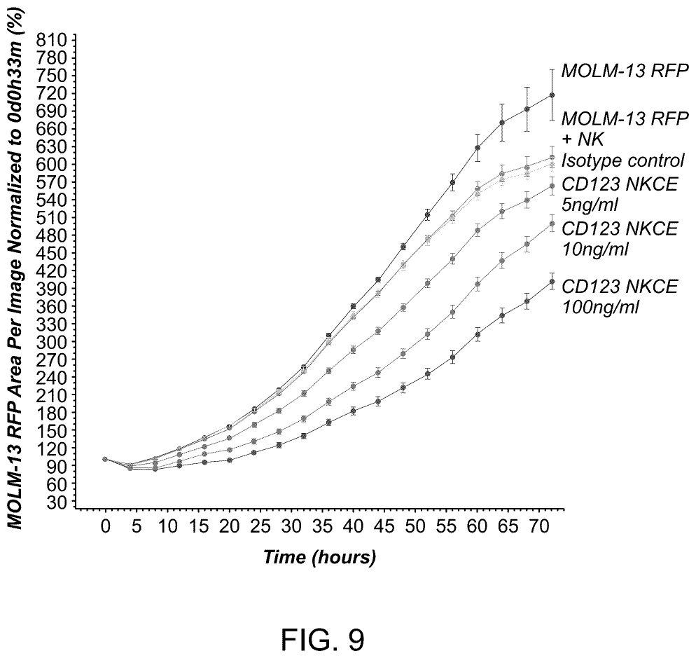

For the combination studies, suboptimal concentrations of CD123 NKCE were selected to be able to better assess the potential benefit of the different combined treatments in terms of tumor cell killing. For this purpose, increasing concentrations of 5, 10 and 100 ng/ml of CD123 NKCE were tested. CD123 NKCE leads to cytotoxic effects against MOLM-13 RFP cells as compared to treatment with isotype control with sub-optimal activity (FIG. 9), allowing further combination with AZA and/or VEN.

B6. CD123 NKCE Combination with 5-AZACYTIDINE and VENETOCLAX Leads to Better Cytotoxic Activity Towards AML Cells as Compared to Combinations with AZA or VEN Alone

For combination studies with CD123 NKCE, the sub-optimal concentration of AZA selected was 600 nM which corresponds to the plasma peak concentration observed in patients treated at the dose of 75 mg/m2, (Derissen E J et al. (2013), Oncologist, vol. 18(5):619-24) while the sub-optimal concentration of VEN was 2 nM, inferior to the peak plasma concentration observed in patients. The plasma concentration of VEN (2 μM at the dose of 400 mg) cannot be used for combination studies as it already induces a very strong killing activity as a single agent (FIG. 1). For CD123 NKCE, suboptimal concentrations of 5 and 100 ng/mL were chosen.

In the presence of the isotype control at 100 ng/mL, no or very low cytotoxicity was observed against MOLM-13 RFP cells over 64 h (time point common to all the 4 tested donors).

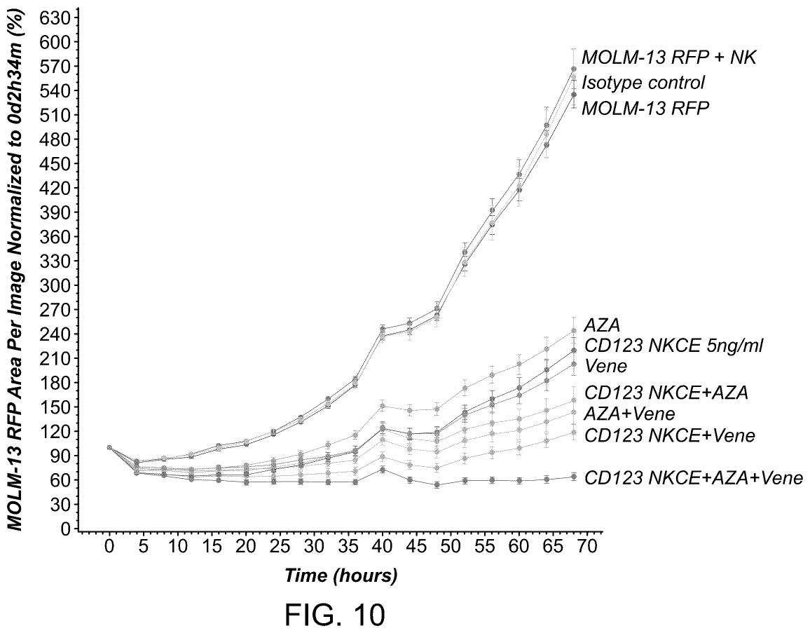

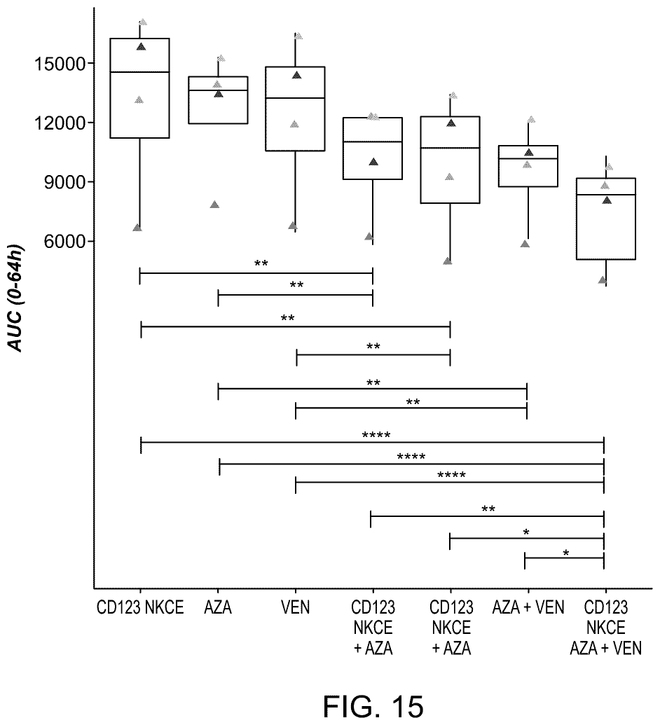

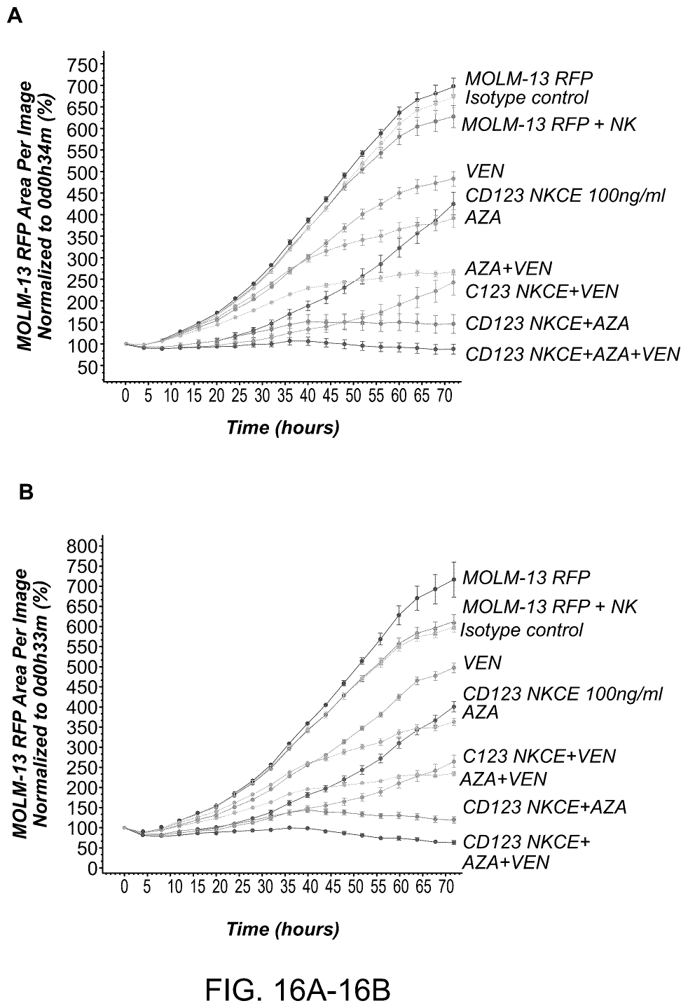

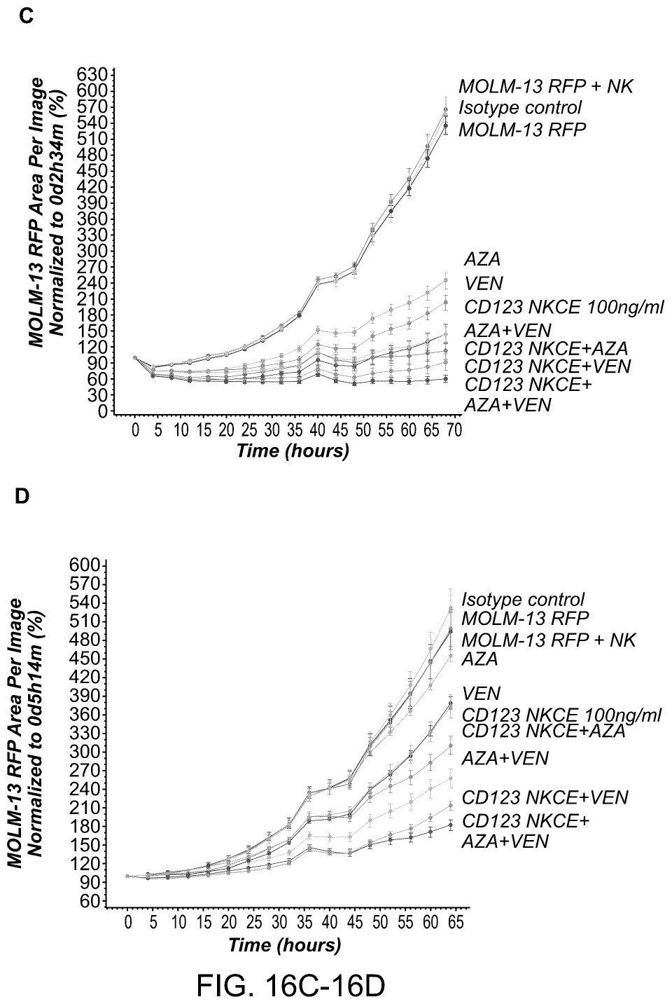

The addition of a single agent (CD123 NKCE or AZA or VEN) at above-defined concentrations led to increased target cell lysis. The combination of two agents (CD123 NKCE+AZA, CD123 NKCE+VEN or AZA+VEN) induced a significant increase in MOLM-13 RFP cell killing as shown in Table 1 below. Moreover, the combination of the three agents resulted in a significantly enhanced cytotoxic effect as compared to each single agent or to the double combinations. See Table 2 below and corresponding FIG. 10 and FIG. 15 for the CD123 NKCE 5 ng/mL and FIG. 16A-D for the CD123 NKCE 100 ng/ml (using different NK donors).

TABLE 1
Comparison of in vitro cytotoxic activity of double combinations versus single
agents

| | | | Adjusted |
|---|---|---|---|
| Comparison | Difference | 97.5% CI | p-value |
| CD123 NKCE 5 ng/ml + AZA 600 nM versus CD123 NKCE | −2955.50 | [−Infty ; −1259.24] | 0.0036 |
| ng/ml | | | |
| CD123 NKCE 5 ng/ml + AZA 600 nM versus AZA 600 nM | −2385.00 | [−Infty; −688.74] | 0.0069 |
| CD123 NKCE 5 ng/ml + VEN 2 nM versus SAR443579 5 | −3289.25 | [−Infty; −1592.99] | 0.0021 |
| ng/ml | | | |
| CD123 NKCE 5 ng/ml + VEN 2 nM versus Venetoclax 2 nM | −2463.50 | [−Infty; −767.24] | 0.0069 |
| AZA 600 nM + VEN 2 nM versus AZA 600 nM | −3043.62 | [−Infty; −1347.37] | 0.0035 |
| AZA 600 nM + VEN 2 nM versus VEN 2 nM | −2788.37 | [−Infty; −1092.12] | 0.0042 |

Adjusted p-value obtained using one-way ANOVA with agent as fixed effect and donor and donor * agent as random effects on AUC.
It was followed by a Bonferroni-Holm adjustment for multiplicity to compare the double combinations versus the single agents.
CI: confidence interval. Confidence intervals for one-sided lower-tail tests will always have a lower bound of −infinity (no lower bound)
Significant adjusted p-value corresponds to a difference significantly lower than 0.
One sided p-values significant at level 2.5% are presented in bold.

TABLE 2
Comparison of in vitro cytotoxic activity of the triple combination versus
double combinations and versus single agents

| | | | Adjusted |
|---|---|---|---|
| Comparison | Difference | 97.5% CI | p-value |
| CD123 NKCE 5 ng/ml + AZA 600 nM + VEN 2 nM versus | −2548.19 | [−Infty; −853.85] | 0.0081 |
| CD123 NKCE 5 ng/ml + AZA 600 nM | | | |
| CD123 NKCE 5 ng/ml + AZA 600 nM + VEN 2 nM versus | −2214.44 | [−Infty; −520.1] | 0.0133 |
| CD123 NKCE 5 ng/ml + VEN 2 nM | | | |
| CD123 NKCE 5 ng/ml + AZA 600 nM + VEN 2 nM versus AZA | −1889.57 | [−Infty; −195.23] | 0.0154 |
| 600 nM + VEN 2 nM | | | |
| CD123 NKCE 5 ng/ml + AZA 600 nM + VEN 2 nM versus | −5503.69 | [−Infty; −3809.35] | <.0001 |
| SAR443579 5 ng/ml | | | |
| CD123 NKCE 5 ng/ml + AZA 600 nM + VEN 2 nM versus AZA | −4933.19 | [−Infty; −3238.85] | <.0001 |
| 600 nM | | | |
| CD123 NKCE 5 ng/ml + AZA 600 nM + VEN 2 nM versus VEN | −4677.94 | [−Infty; −2983.6] | <.0001 |
| 2 nM | | | |

Adjusted p-value obtained using one-way ANOVA with agent as fixed effect and donor and donor * agent as random effects on AUC.
It was followed by a Bonferroni-Holm adjustment for multiplicity to compare the triple combination versus the single agents and double combinations.
CI: confidence interval. Confidence intervals for one-sided lower-tail tests will always have a lower bound of −infinity (no lower bound)
Significant adjusted p-value corresponds to a difference significantly lower than 0.
One sided p-values significant at level 2.5% are presented in bold.

In parallel to the functional analysis, cell viability and CD123 expression were assessed for MOLM-13 cells over 72 h (time of the cytotoxicity assays) and 6 days of coculture with NK cells in the presence of AZA or VEN. The effects of AZA at 600 nM and 3 μM and of VEN at 2, 5 and 10 nM were evaluated by flow cytometry in 3 independent experiments.

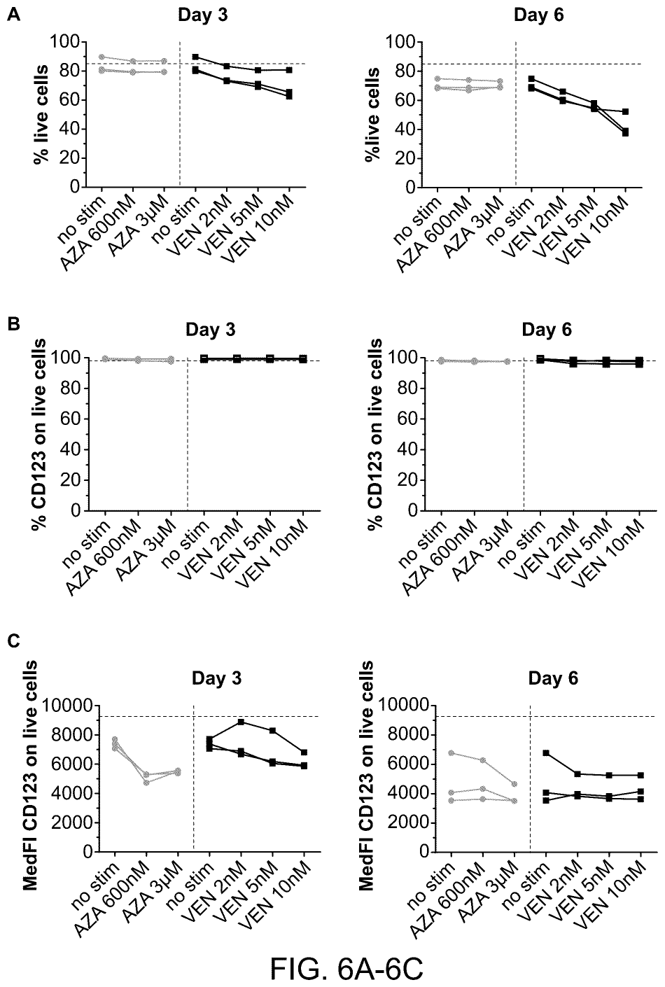

AZA did not affect MOLM-13 cell viability at the tested concentrations. VEN impacted MOLM-13 viability mainly after 6 days of treatment in a concentration-dependent manner (FIG. 6A, left column). The expression of CD123 on MOLM-13 at the two time points was not impacted by any agent (FIG. 6B).

### B7. Conclusion

As demonstrated in the previous Examples, AZA and VEN have minimal impact on CD123 target expression on MOLM-13 AML tumor cells (FIG. 3A-3B, FIG. 5A-5C 5, FIG. 6A-6C) and no impact on CD16a and NKp46 expression on NK cells from healthy donors (FIG. 7A-7C and FIG. 8A-8C), at the tested concentrations and up to 6 days of treatment, thus being suitable for combination with the CD123 NKCE. A cytotoxic activity against MOLM-13 AML tumor cells was measured with AZA and VEN at the concentration of 10 μM and from 0.01 nM to 10 μM respectively, while no cytotoxic effect was observed on NK cells at the same concentrations.

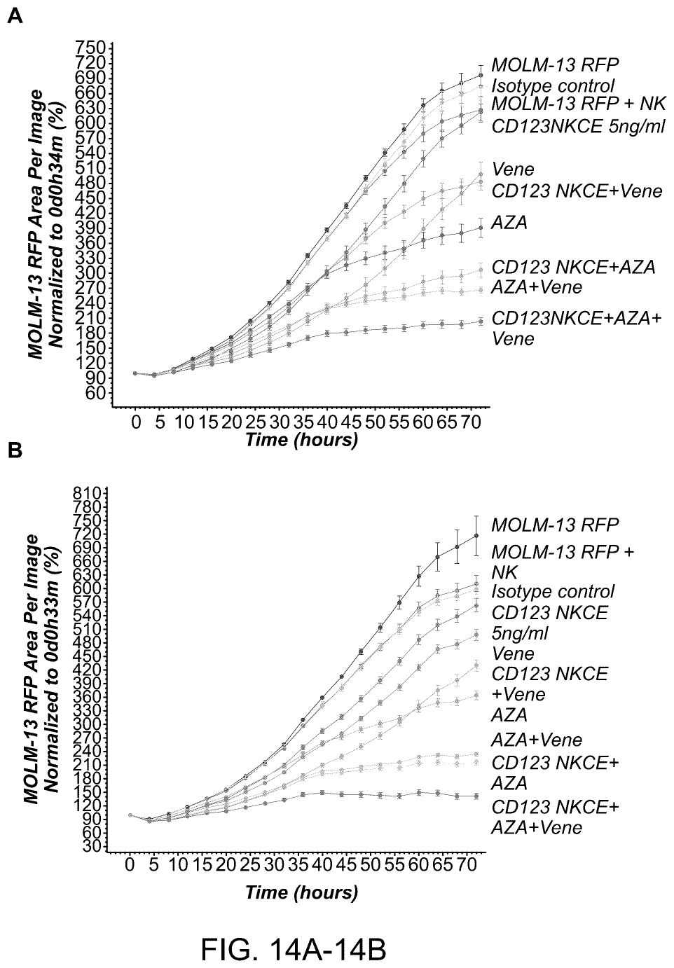

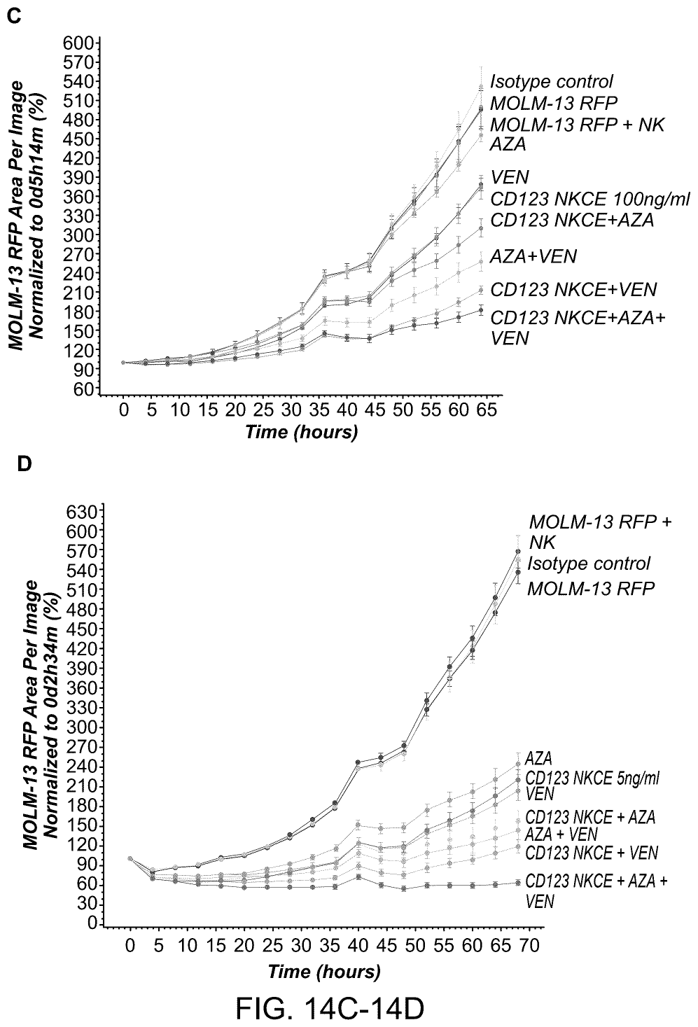

The coculture of NK cells with MOLM-13 AML tumor cells in the presence of CD123 NKCE at 5 ng/mL, AZA at 600 nM or VEN at 2 nM led to some target lysis when administered as a monotherapy. See target lysis results of each single agent in FIG. 10, FIG. 14 as well as Table 1. In contrast, when a CD123 NKCE was combined with either AZA or VEN at the same concentration, target lysis was significantly increased exhibiting a synergistic effect (CD123 NKCE+AZA, CD123 NKCE+VEN or AZA+VEN) (FIG. 10, FIG. 14, and Table 2). The target lysis of the CD123 NKCE+AZA+VEN triple combination resulted in the most significantly enhanced cytotoxic activity and the greatest synergistic effect relative to the different double combinations and each single agent alone (FIG. 10, FIG. 14, and Table 2).

1. A method for treating or preventing a leukemia, a myelodysplastic syndrome (MDS) or other hematological neoplastic disorder in a subject in need thereof, said method comprising administering to the subject an effective amount of a combination comprising: (i) a multifunctional binding protein comprising a first antigen binding domain (ABD) with binding specificity to CD123 (an antigen of interest on tumoral target cells) and a second (ABD) with binding specificity to NKp46 (a surface biomarker on immune NK cells); and one or both of: (ii) a BCL-2 inhibitor, and (iii) a DNA hypomethylating agent.

2. The method of claim 1, wherein the binding protein further comprises all or part of an immunoglobulin Fc variant region or variant thereof, optionally wherein all or part of the Fc region or variant thereof binds to a human Fc-γ receptor.

3. (canceled)

4. The method of claim 1, wherein the first ABD binds specifically to human CD123 and comprises:
(i) a heavy chain variable domain (VH1) comprising a CDR-H1, H2, and H3 corresponding to the amino acid sequences of SEQ ID NO: 1, 2, and 3, respectively or corresponding to the amino acid sequences of SEQ ID NO: 4, 5, and 6, respectively; and
(ii) a light chain variable domain (VL1) comprising a CDR-L1, L2, and L3 corresponding to the amino acid sequences of SEQ ID NO: 7, 8, and 9, respectively or corresponding to the amino acid sequences of SEQ ID NO: 10, 11, and 12, respectively, optionally wherein:
a) the VH1 comprises an amino acid sequence that is at least about 90% identical to the amino acid sequence of SEQ ID NO: 41, and wherein the VL1 comprises an amino acid sequence that is at least about 90% identical to the amino acid sequence of SEQ ID NO: 43; or
b) the VH1 comprises an amino acid sequence that is at least about 90% identical to the amino acid sequence of SEQ ID NO: 42, and wherein the VL1 comprises an amino acid sequence that is at least about 90% identical to the amino acid sequence of SEQ ID NO: 44.

5-6. (canceled)

7. The method of claim 1, wherein the second ABD binds specifically human NKp46 and comprises:
(i) a VH2 comprising a CDR-H1, 2 and 3 corresponding to:
the amino acid sequences of SEQ ID NO: 13, 14, and 15 respectively;
the amino acid sequences of SEQ ID NO: 16, 17, and 18 respectively;
the amino acid sequences of SEQ ID NO: 19, 20, and 21 respectively;
the amino acid sequences of SEQ ID NO: 22, 23, and 24 respectively; or
the amino acid sequences of SEQ ID NO: 16, 25 and 26 respectively; and
(ii) a VL2 comprising a CDR-L1, 2 and 3 corresponding to:
the amino acid sequences of SEQ ID NO: 27, 28, and 29 respectively;
the amino acid sequences of SEQ ID NO: 30, 31, and 32 respectively;
the amino acid sequences of SEQ ID NO: 33, 34, and 35 respectively;
the amino acid sequences of SEQ ID NO: 36, 37, and 38 respectively; or
the amino acid sequences SEQ ID NO: 39, 31 and 40 respectively, optionally wherein:
a) the VH2 comprises an amino acid sequence that is at least about 90% identical to the amino acid sequence of SEQ ID NO: 45, and wherein the VL1 comprises an amino acid sequence that is at least about 90% identical to the amino acid sequence of SEQ ID NO: 53; b) the VH2 comprises an amino acid sequence that is at least about 90% identical to the amino acid sequence of SEQ ID NO: 46, and wherein the VL2 comprises an amino acid sequence that is at least about 90% identical to the amino acid sequence of SEQ ID NO: 54; c) the VH2 comprises an amino acid sequence that is at least about 90% identical to the amino acid sequence of SEQ ID NO: 47, and wherein the VL2 comprises an amino acid sequence that is at least about 90% identical to the amino acid sequence of SEQ ID NO: 55; d) the VH2 comprises an amino acid sequence that is at least about 90% identical to the amino acid sequence of SEQ ID NO: 48, and wherein the VL2 comprises an amino acid sequence that is at least about 90% identical to the amino acid sequence of SEQ ID NO: 56; e) the VH2 comprises an amino acid sequence that is at least about 90% identical to the amino acid sequence of SEQ ID NO: 49, and wherein the VL2 comprises an amino acid sequence that is at least about 90% identical to the amino acid sequence of SEQ ID NO: 57; f) the VH2 comprises an amino acid sequence that is at least about 90% identical to the amino acid sequence of SEQ ID NO: 50, and wherein the VL2 comprises an amino acid sequence that is at least about 90% identical to the amino acid sequence of SEQ ID NO: 58; g) the VH2 comprises an amino acid sequence that is at least about 90% identical to the amino acid sequence of SEQ ID NO: 51, and wherein the VL2 comprises an amino acid sequence that is at least about 90% identical to the amino acid sequence of SEQ ID NO: 59; or
h) the VH2 comprises an amino acid sequence that is at least about 90% identical to the amino acid sequence of SEQ ID NO: 52, and wherein the VL2 comprises an amino acid sequence that is at least about 90% identical to the amino acid sequence of SEQ ID NO: 60.

8-9. (canceled)

10. The method of claim 1, wherein the binding protein comprises three polypeptide chains (I), (II) and (III) that form two ABDs, as defined below:
V_{1A}-C_{1A}-Hinge₁-(C_{H}2-C_{H}3)_{A} (I)
V_{1B}-C_{1B}-Hinge₂-(C_{H}2-C_{H}3)_{B}-L₁-V_{2A}-C_{2A}-Hinge₃ (II)
V_{2B}-C_{2B} (III)
wherein:
V_{1A} and V_{1B} form a binding pair V₁ (V_{H1}/V_{L1});
V_{2A} and V_{2B} form a binding pair V₂ (V_{H2}/V_{L2});
C_{1A} and C_{1B} form a pair C₁ (C_{H}1/C_{L}) and C_{2A} and C_{2B} form a pair C₂ (C_{H}1/C_{L}) wherein C_{H}1 is an immunoglobulin heavy chain constant domain 1 and C_{L} is an immunoglobulin light chain constant domain;
Hinge₁, Hinge₂ and Hinge₃ are identical or different and correspond to all or part of an immunoglobulin hinge region;
(C_{H}2-C_{H}3)_{A} and (C_{H}2-C_{H}3)_{B} are identical or different, and comprise an immunoglobulin heavy chain constant domain 2 (C_{H}2) and an immunoglobulin heavy chain constant domain 3 (C_{H}3); and
L₁ is an amino acid linker, optionally wherein:
C_{1B} is an immunoglobulin heavy chain constant domain 1 (C_{H}1); C_{2A} is an immunoglobulin heavy chain constant domain 1 (C_{H}1); C_{L} corresponds to an immunoglobulin kappa light chain constant domain (C_{κ}); (C_{H}2-C_{H}3)_{A} corresponds to the amino acid sequence of SEQ ID NO: 69; (C_{H}2-C_{H}3)_{B} corresponds to the amino acid sequence of SEQ ID NO: 70; Hinge₁ corresponds to the amino acid sequence of SEQ ID NO: 74; Hinge₂ corresponds to the amino acid sequence of SEQ ID NO: 75; Hinge₃ corresponds to the amino acid sequence of SEQ ID NO: 77; and L₁ corresponds to the amino acid sequence of SEQ ID NO: 76;
the binding protein comprises at least two polypeptide chains linked by at least one disulfide bridge;
the polypeptide chains (I) and (II) are linked by at least one disulfide bridge between C_{1A} and Hinge₂ and/or wherein the polypeptide chains (II) and (III) are linked by at least one disulfide bridge between Hinge₃ and C_{2B}; and/or
V_{1A} is V_{L1} and V_{1B} is V_{H1} and V_{2A} is V_{H2} and V_{2B} is V_{L2}.

11-18. (canceled)

19. The method of claim 10, wherein:
(i) polypeptide (I) comprises or consists of an amino acid sequence of SEQ ID NO: 64;
(ii) polypeptide (II) comprises or consists of an amino acid sequence of SEQ ID NO: 65; and
(iii) polypeptide (III) comprises or consists of an amino acid sequence of SEQ ID NO: 66.

20. The method of claim 1, wherein the BCL-2 inhibitor is venetoclax, oblimersen (e.g., a sodium salt of oblimersen, also known as oblimersen sodium), obatoclax mesylate, palcitoclax, or LP-118.

21. (canceled)

22. The method of claim 1, wherein the DNA hypomethylating agent is a 5-azacytidine, a cytidine, a decitabine, or a guadecitabine.

23. (canceled)

24. The method of claim 1, wherein the method comprises administering to the subject an effective amount of a combination comprising: (i) a binding protein comprising a first antigen binding domain (ABD) with binding specificity to CD123 and a second (ABD) with binding specificity to NKp46; (ii) a BCL-2 inhibitor; and (iii) a DNA hypomethylating agent.

25. The method of claim 1, wherein the leukemia is acute myeloid leukemia (AML), optionally wherein the AML is relapsed or refractory, the AML CD123 positive, the AML is newly diagnosed AML, wherein the patient is ineligible for intensive chemotherapy, the AML is CD123 positive, newly diagnosed AML and wherein the subject is ineligible for intensive chemotherapy, and/or the subject has a confirmed diagnosis of AML with positive CD123 tumor expression and ineligible for intensive chemotherapy.

26-30. (canceled)

31. The method of claim 1, wherein the leukemia is B cell acute lymphoblastic leukemia (B-ALL).

32. The method of claim 1, wherein the myelodysplastic syndrome is a high-risk myelodysplastic syndrome (HR-MDS).

33. (canceled)

34. The method of claim 1, wherein the combination results in an increase in target cell lysis in the subject relative to administering any one of the binding protein, the BCL-2 inhibitor, or the DNA hypomethylating agent alone to the subject, optionally the combination results in a 1-fold, a 2-fold, a 3-fold, a 4-fold, a 5-fold, a 6-fold, or a 7-fold increase in target cell lysis in the subject relative to administering any one of the binding protein, the BCL-2 inhibitor, or the DNA hypomethylating agent alone to the subject.

35-36. (canceled)

37. A method of treating acute myeloid leukemia (AML) in a subject in need thereof, said method comprising administering to the subject an effective amount of a combination comprising:
(i) a binding protein comprising a first ABD with binding specificity to CD123 and a second ABD with binding specificity to NKp46,
wherein the first ABD comprises a VH1 and a VL1, wherein:
the VH1 comprises a complementary determining region (CDR)-H1, H2 and H3 corresponding to the amino acid sequences of SEQ ID NO: 1, 2, and 3; and
the VL1 comprises a CDR-L1, L2 and L3 corresponding to the amino acid sequences of SEQ ID NO: 7, 8, and 9;
wherein the second antigen binding domain comprises a heavy chain variable domain (VH2) and a light chain variable domain (VL2), wherein:
the VH2 comprises a CDR-H1, H2, and H3 corresponding to the amino acid sequences of SEQ ID NO: 13, 14, and 15;
the VL2 comprises a CDR-L1, L2, and L3 corresponding to the amino acid sequences of SEQ ID NO: 27, 28, and 29; and
wherein all or part of the immunoglobulin Fc region or variant thereof binds to a human Fc-γ receptor;
(ii) a venetoclax or a pharmaceutically acceptable salt thereof; and
(iii) a 5-azacytidine or a pharmaceutically acceptable salt thereof,
wherein the AML is newly diagnosed AML, the subject has positive CD123 tumor expression, and wherein the subject is ineligible for intensive chemotherapy.

38-39. (canceled)

40. The method of claim 1, wherein the binding protein is administered to the subject intravenously, subcutaneously, intraperitoneally, or intramuscularly.

41. (canceled)

42. The method of claim 1, wherein the binding protein, the BCL-2 inhibitor, and the DNA hypomethylating agent are administered simultaneously or administered sequentially.

43. (canceled)

44. The method of claim 1, wherein the binding protein is administered to a subject for at least one cycle, at least two cycles, or at least three cycles.

45-46. (canceled)

47. An effective amount of a combination comprising:
(i) a binding protein comprising a first antigen binding domain (ABD) with binding specificity to CD123 and a second (ABD) with binding specificity to NKp46; and one or both of:
(ii) a BCL-2 inhibitor, and
(iii) a DNA hypomethylating agent
for use in a method for treating or preventing a leukemia or a myelodysplastic syndrome in a subject in need thereof.

48-49. (canceled)


SEQUENCE LISTING (117 sequences)

SEQ ID NO: 1 - protein, 10 aa, synthetic construct
GYSFTDYYMK

SEQ ID NO: 2 - protein, 10 aa, synthetic construct
DIIPSSGATF

SEQ ID NO: 3 - protein, 11 aa, synthetic construct
SHLLRASWFAY

SEQ ID NO: 4 - protein, 8 aa, synthetic construct
GFTFSHYN

SEQ ID NO: 5 - protein, 8 aa, synthetic construct
ITYDDHST

SEQ ID NO: 6 - protein, 10 aa, synthetic construct
ARLVNYAFAY

SEQ ID NO: 7 - protein, 17 aa, synthetic construct
ESSQSLLSSGNQKNYLT

SEQ ID NO: 8 - protein, 7 aa, synthetic construct
WASTRES

SEQ ID NO: 9 - protein, 9 aa, synthetic construct
QNDYSYPYT

SEQ ID NO: 10 - protein, 6 aa, synthetic construct
QTVGNN

SEQ ID NO: 11 - skipped

SEQ ID NO: 12 - protein, 8 aa, synthetic construct
QRMYNSPT

SEQ ID NO: 13 - protein, 5 aa, synthetic construct
DYVIN

SEQ ID NO: 14 - protein, 17 aa, synthetic construct
EIYPGSGTNYYNEKFKA

SEQ ID NO: 15 - protein, 11 aa, synthetic construct
RGRYGLYAMDY

SEQ ID NO: 16 - protein, 6 aa, synthetic construct
SDYAWN

SEQ ID NO: 17 - protein, 16 aa, synthetic construct
YITYSGSTSYNPSLES

SEQ ID NO: 18 - protein, 13 aa, synthetic construct
GGYYGSSWGVFAY

SEQ ID NO: 19 - protein, 5 aa, synthetic construct
EYTMH

SEQ ID NO: 20 - protein, 17 aa, synthetic construct
GISPNIGGTSYNQKFKG

SEQ ID NO: 21 - protein, 7 aa, synthetic construct
RGGSFDY

SEQ ID NO: 22 - protein, 5 aa, synthetic construct
SFTMH

SEQ ID NO: 23 - protein, 17 aa, synthetic construct
YINPSSGYTEYNQKFKD

SEQ ID NO: 24 - protein, 8 aa, synthetic construct
GSSRGFDY

SEQ ID NO: 25 - protein, 16 aa, synthetic construct
YITYSGSTNYNPSLKS

SEQ ID NO: 26 - protein, 11 aa, synthetic construct
CWDYALYAMDC

SEQ ID NO: 27 - protein, 11 aa, synthetic construct
RASQDISNYLN

SEQ ID NO: 28 - protein, 7 aa, synthetic construct
YTSRLHS

SEQ ID NO: 29 - protein, 9 aa, synthetic construct
QQGNTRPWT

SEQ ID NO: 30 - protein, 11 aa, synthetic construct
RVSENIYSYLA

SEQ ID NO: 31 - protein, 7 aa, synthetic construct
NAKTLAE

SEQ ID NO: 32 - protein, 9 aa, synthetic construct
QHHYGTPWT

SEQ ID NO: 33 - protein, 11 aa, synthetic construct
RASQSISDYLH

SEQ ID NO: 34 - protein, 7 aa, synthetic construct
YASQSIS

SEQ ID NO: 35 - protein, 9 aa, synthetic construct
QNGHSFPLT

SEQ ID NO: 36 - protein, 11 aa, synthetic construct
RASENIYSNLA

SEQ ID NO: 37 - protein, 7 aa, synthetic construct
AATNLAD

SEQ ID NO: 38 - protein, 9 aa, synthetic construct
QHFWGTPRT

SEQ ID NO: 39 - protein, 11 aa, synthetic construct
RTSENIYSYLA

SEQ ID NO: 40 - protein, 9 aa, synthetic construct
QHHYDTPLT

SEQ ID NO: 41 - protein, 120 aa, synthetic construct [sequence omitted]

SEQ ID NO: 42 - protein, 117 aa, synthetic construct
EVQLVESGGGLVQPGRSLKLSCAASGFTFSHYNMAWVRQAPKKGLEWVATITYDDHSTYYRDSVKGRFTISRDTAKSTLYLQMDSLRSEDTATYYCARLVNYAFAYWGQGTLVTVSS

SEQ ID NO: 43 - protein, 113 aa, synthetic construct [sequence omitted]

SEQ ID NO: 44 - protein, 106 aa, synthetic construct
NIVMTQSPKSMSISVGDRVTMNCKASQTVGNNIAWYQQKPGLSPQLLIDYASNRYTGVPNRFTGGGYGTDFILTINSVQAEDAAFYYCQRMYNSPTFGGGTKLELK

SEQ ID NO: 45 - protein, 120 aa, synthetic construct
QVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSS

SEQ ID NO: 46 - protein, 120 aa, synthetic construct
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSS

SEQ ID NO: 47 - protein, 122 aa, synthetic construct
EVQLQESGPGLVKPSQSLSLTCTVTGYSITSDYAWNWIRQFPGNKLEWMGYITYSGSTSYNPSLESRISITRDTSTNQFFLQLNSVTTEDTATYYCARGGYYGSSWGVFAYWGQGTLVTVSA

SEQ ID NO: 48 - protein, 116 aa, synthetic construct
EVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSS

SEQ ID NO: 49 - protein, 117 aa, synthetic construct
QVQLQQSAVELARPGASVKMSCKASGYTFTSFTMHWVKQRPGQGLEWIGYINPSSGYTEYNQKFKDKTTLTADKSSSTAYMQLDSLTSDDSAVYYCVRGSSRGFDYWGQGTLVTVSA

SEQ ID NO: 50 - protein, 122 aa, synthetic construct
QVQLQESGPGLVKPSQTLSLTCTVSGYSISSDYAWNWIRQPPGKGLEWIGYITYSGSTSYNPSLESRVTISRDTSKNQFSLKLSSVTAADTAVYYCARGGYYGSSWGVFAYWGQGTLVTVSS

SEQ ID NO: 51 - protein, 117 aa, synthetic construct
QVQLVQSGAEVKKPGASVKVSCKASGYTFTSFTMHWVRQAPGQGLEWIGYINPSSGYTEYNQKFKDRVTITADKSTSTAYMELSSLRSEDTAVYYCVRGSSRGFDYWGQGTLVTVSS

SEQ ID NO: 52 - protein, 120 aa, synthetic construct
QVQLQESGPGLVKPSQTLSLTCTVSGYSISSDYAWNWIRQPPGKGLEWIGYITYSGSTNYNPSLKSRVTISRDTSKNQFSLKLSSVTAADTAVYYCARCWDYALYAMDCWGQGTTVTVSS

SEQ ID NO: 53 - protein, 107 aa, synthetic construct
DIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIK

SEQ ID NO: 54 - protein, 107 aa, synthetic construct
DIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIK

SEQ ID NO: 55 - protein, 107 aa, synthetic construct
DIQMTQSPASLSASVGETVTITCRVSENIYSYLAWYQQKQGKSPQLLVYNAKTLAEGVPSRFSGSGSGTQFSLKINSLQPEDFGSYYCQHHYGTPWTFGGGTKLEIK

SEQ ID NO: 56 - protein, 107 aa, synthetic construct
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 57 - protein, 107 aa, synthetic construct
DIQMIQSPASLSVSVGETVTITCRASENIYSNLAWFQQKQGKSPQLLVYAATNLADGVPSRFSGSGSGTQYSLKINSLQSEDFGIYYCQHFWGTPRTFGGGTKLEIK

SEQ ID NO: 58 - protein, 107 aa, synthetic construct
DIQMTQSPSSLSASVGDRVTITCRVSENIYSYLAWYQQKPGKAPKLLVYNAKTLAEGVPSRFSGSGSGTDFTLTISSLQPEDFATYYCQHHYGTPWTFGGGTKVEIK

SEQ ID NO: 59 - protein, 107 aa, synthetic construct
DIQMTQSPSSLSASVGDRVTITCRASENIYSNLAWFQQKPGKAPKLLVYAATNLADGVPSRFSGSGSGTDYTLTISSLQPEDFATYYCQHFWGTPRTFGGGTKVEIK

SEQ ID NO: 60 - protein, 107 aa, synthetic construct
DIQMTQSPSSLSASVGDRVTITCRTSENIYSYLAWCQQKPGKAPKLLIYNAKTLAEGVPSRFSGSGSGTDFTLTISSLQPEDFATYYCQHHYDTPLTFGQGTKLEIK

SEQ ID NO: 61 - protein, 467 aa, synthetic construct
MSVPTQVLGLLLLWLTDARCDIVMTQSPDSLAVSLGERATINCESSQSLLSSGNQKNYLTWYQQKPGQPPKPLIYWASTRESGVPDRFSGSGSGTDFTLTISSLQAEDVAVYYCQNDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 700 aa, synthetic construct
MEWSWVFLFFLSVTTGVHSEVQLVQSGAEVKKPGESLKISCKGSGYSFTDYYMKWARQMPGKGLEWMGDIIPSSGATFYNQKFKGQVTISADKSISTTYLQWSSLKASDTAMYYCARSHLLRASWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHS

SEQ ID NO: 63 - protein, 234 aa, synthetic construct
MSVPTQVLGLLLLWLTDARCDIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 447 aa, synthetic construct
DIVMTQSPDSLAVSLGERATINCESSQSLLSSGNQKNYLTWYQQKPGQPPKPLIYWASTRESGVPDRFSGSGSGTDFTLTISSLQAEDVAVYYCQNDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 681 aa, synthetic construct
EVQLVQSGAEVKKPGESLKISCKGSGYSFTDYYMKWARQMPGKGLEWMGDIIPSSGATFYNQKFKGQVTISADKSISTTYLQWSSLKASDTAMYYCARSHLLRASWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHS

SEQ ID NO: 66 - protein, 214 aa, synthetic construct
DIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 107 aa, synthetic construct [sequence omitted]

SEQ ID NO: 68 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 69 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 110 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 72 - protein, 107 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 106 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 10 aa, synthetic construct
DKTHTCPPCP

SEQ ID NO: 75 - protein, 15 aa, synthetic construct
EPKSCDKTHTCPPCP

SEQ ID NO: 76 - protein, 4 aa, synthetic construct
STGS

SEQ ID NO: 77 - protein, 10 aa, synthetic construct
EPKSCDKTHS

SEQ ID NO: 78 - protein, 9 aa, synthetic construct
EPKSCDKTH

SEQ ID NO: 79 - protein, 10 aa, synthetic construct
EPKSCDKTHT

SEQ ID NO: 80 - protein, 255 aa, Homo sapiens
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQN

SEQ ID NO: 81 - protein, 262 aa, Macaca fascicularis
MSSTLRALLCLGLCLSQRISAPKQTLPKPIIRAESTYMVPKEKQATLCCQGSYGAVEYQLHFEGSLFAVERPKPPERINGVKFHIPDMNSRKAGRYSCIYRVGELWSERSDLLDLVVTEMYDTPTLSVHPGPEVTSGEKVTFYCRLDTATSMFLLLKEGRSRDVQRSYGKVQAEFPMGPVTTAHRGSYRCFGSYNNYAWSFPSEPVKLLVTGDIENTSLAPTDPTFPDSWDTCLLTRETGLQKDLALWDHTAQNDYKDDDDK

SEQ ID NO: 82 - protein, 287 aa, Homo sapiens
TKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWR

SEQ ID NO: 83 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 84 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 85 - protein, 441 aa, synthetic construct
EIVLTQSPATLSLSPGERATLSCRASQSVRSYLAWYQQKPGQAPRLLFSDASNRATGIPARFSGSGSGTDFTLTISSLEPEDFAVYYCQQYRYSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 678 aa, synthetic construct
EVQLVESGGGLVQPGRSLRLSCAASGFTFDNYAMHWVRQAPGKGLEWVSGISRSSGDIDYADSVKGRFTISRDNAKNSLYLQMNSLRAEDTALYYCARGGVGSFDTWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHS

SEQ ID NO: 87 - protein, 214 aa, synthetic construct; identical to SEQ ID NO: 66

SEQ ID NO: 88 - protein, 447 aa, synthetic construct; identical to SEQ ID NO: 64

SEQ ID NO: 89 - protein, 680 aa, synthetic construct
EVQLVQSGAEVKKPGESLKISCKGSGYSFTDYYMKWARQMPGKGLEWMGDIIPSSGATFYNQKFKGQVTISADKSISTTYLQWSSLKASDTAMYYCARSHLLRASWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLVESGGGLVKPGGSLKLSCAASGFTFSDYGMHWVRQAPEKGLEWVAYISSGSSTIYYADTVKGRFTISRDNAKNTLFLQMTSLRSEDTAMYYCARGTTIFNYFEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHS

SEQ ID NO: 90 - protein, 220 aa, synthetic construct
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYEIPPTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 447 aa, synthetic construct; identical to SEQ ID NO: 64

SEQ ID NO: 92 - protein, 680 aa, synthetic construct
EVQLVQSGAEVKKPGESLKISCKGSGYSFTDYYMKWARQMPGKGLEWMGDIIPSSGATFYNQKFKGQVTISADKSISTTYLQWSSLKASDTAMYYCARSHLLRASWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 215 aa, synthetic construct
DIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHS

SEQ ID NO: 94 - protein, 447 aa, synthetic construct; identical to SEQ ID NO: 64

SEQ ID NO: 95 - protein, 679 aa, synthetic construct
EVQLVQSGAEVKKPGESLKISCKGSGYSFTDYYMKWARQMPGKGLEWMGDIIPSSGATFYNQKFKGQVTISADKSISTTYLQWSSLKASDTAMYYCARSHLLRASWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLVQSGAEVKKSGESLKISCKGSGYSFTSYWIGWVRQMPGKGLEWMGIFYPGDSSTRYSPSFQGQVTISADKSVNTAYLQWSSLKASDTAMYYCARRRNWGNAFDIWGQGTMVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 216 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSTWTFGQGTKVEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHS

SEQ ID NO: 97 - protein, 214 aa, synthetic construct
EIVLTQSPATLSLSPGERATLSCRASQSVRSYLAWYQQKPGQAPRLLFSDASNRATGIPARFSGSGSGTDFTLTISSLEPEDFAVYYCQQYRYSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 447 aa, synthetic construct
EVQLVESGGGLVQPGRSLRLSCAASGFTFDNYAMHWVRQAPGKGLEWVSGISRSSGDIDYADSVKGRFTISRDNAKNSLYLQMNSLRAEDTALYYCARGGVGSFDTWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 6 aa, synthetic construct
QDISNY

SEQ ID NO: 100 - skipped

SEQ ID NO: 101 - protein, 10 aa, synthetic construct
QQGNTRPWTF

SEQ ID NO: 102 - protein, 8 aa, synthetic construct
GYTFSDYV

SEQ ID NO: 103 - protein, 8 aa, synthetic construct
IYPGSGTN

SEQ ID NO: 104 - protein, 13 aa, synthetic construct
ARRGRYGLYAMDY

SEQ ID NO: 105 - protein, 7 aa, synthetic construct
SQDISNY

SEQ ID NO: 106 - skipped

SEQ ID NO: 107 - protein, 6 aa, synthetic construct
GNTRPW

SEQ ID NO: 108 - protein, 7 aa, synthetic construct
GYTFSDY

SEQ ID NO: 109 - protein, 4 aa, synthetic construct
PGSG

SEQ ID NO: 110 - protein, 9 aa, synthetic construct
GRYGLYAMD

SEQ ID NO: 111 - protein, 8 aa, synthetic construct
ASQDISNY

SEQ ID NO: 112 - protein, 12 aa, synthetic construct
YTSRLHSGVPSR

SEQ ID NO: 113 - protein, 10 aa, synthetic construct
ASGYTFSDYV

SEQ ID NO: 114 - protein, 17 aa, synthetic construct
IYPGSGTNYYNEKFKAK

SEQ ID NO: 115 - protein, 10 aa, synthetic construct
RGRYGLYAMD

SEQ ID NO: 116 - protein, 9 aa, synthetic construct
QQGNTRPWT

SEQ ID NO: 117 - protein, 4 aa, synthetic construct
CCCC